(19) United States
(12) Patent Application Publication
(10) Pub. No.: US 2006/0025445 A1
(43) Pub. Date: Feb. 2, 2006

(54) 11-Beta HSD1 inhibitors

(76) Inventors: Jason Shaoyun Xiang, Winchester, MA (US); John Caedmon McKew, Arlington, MA (US); Steve Y. Tam, Wellesley, MA (US); Manus Ipek, Watetown, MA (US); Vipin Suri, Waltham, MA (US); Tarek Suhayl Mansour, New City, NY (US)

Correspondence Address: FISH & RICHARDSON P.C., P.O BOX 1022, MINNEAPOLIS, MN, 55440-1022, US

(21) Appl. No.: 11/196,643

(22) Filed: Aug. 2, 2005

Related U.S. Application Data
(60) Provisional application No. 60/598,373, filed on Aug. 2, 2004.

(51) Int. Cl. A61K 31/445; A61K 31/40; A61K 31/381

(52) U.S. Cl. 514/317; 514/471; 514/408; 514/602; 546/229; 548/561; 549/491; 564/86; 549/76

(57) ABSTRACT

This invention relates to inhibiting 11-beta HSD1.

### CROSS REFERENCE TO RELATED APPLICATIONS

This application claims the benefit of U.S. Provisional Application No. 60/598,373, filed on Aug. 2, 2004, which is incorporated herein by reference in its entirety.

### TECHNICAL FIELD

This invention relates to inhibiting 11βHSD1.

### BACKGROUND

Diabetes is generally characterized by relatively high levels of plasma glucose (hyperglycemia) in the fasting state. Patients having type 2 diabetes (non-insulin dependent diabetes mellitus (NIDDM)) produce insulin (and even exhibit hyperinsulinemia), whilst demonstrating hyperglycemia.

Type 2 diabetics can often develop insulin resistance, in which the effect of insulin in stimulating glucose and lipid metabolism is diminished. Further, patients having insulin resistance, but have not developed type 2 diabetes, are also at risk of developing Syndrome X (metabolic syndrome). Syndrome X is characterized by insulin resistance, along with obesity (e.g., abdominal obesity), hyperinsulinemia, high blood pressure, relatively low HDL and relatively high VLDL.

Glucocorticoids (e.g., cortisol in humans, corticosterone in rodents) are counter regulatory hormones that oppose the action of insulin. It is established that glucocorticoid activity is controlled at the tissue level by intracellular interconversion of active cortisol and inactive cortisone by the 11-beta hydroxysteroid dehydrogenases, 11βHSD1, which activates cortisone and 11βHSD2, which inactivates cortisol. Excess levels of glucocorticoids (e.g., cortisol) can cause metabolic complications. For example, excess cortisol is associated with disorders including NIDDM, obesity, dyslipidemia, insulin resistance, and hypertension.

It is believed that inhibition of 11≢2HSD1 can reduce the effects of excessive amounts of 11β-hydroxysteroids, e.g., cortisol, and therefore can be useful for the treatment and control of diseases mediated by abnormally high levels of cortisol and other 11β-hydroxysteroids, e.g., NIDDM, obesity, dyslipidemia, and hypertension.

### SUMMARY

In one aspect, this invention relates to compounds of formula (I): 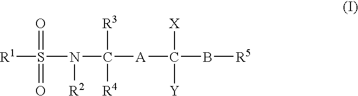
wherein:

each of R¹ and R⁵ is, independently:

(i) C₁-C₂₀ alkyl, optionally substituted with from 1-10 R^{a}; or

(ii) C₃-C₁₆ cycloalkyl, optionally substituted with from 1-10 R^{a}; or

(iii) C₁-C₂₀ haloalkyl or C₃-C₁₆ halocycloalkyl, optionally substituted with from 1-10 R^{a}; or

(iv) C₂-C₂₀ alkenyl, C₂-C₂₀ alkynyl, C₃-C₁₆ cycloalkenyl, heterocyclyl including 3-16 atoms, or heterocycloalkenyl including 3-16 atoms, each of which is optionally substituted with from 1-10 R^{b}; or

(v) C₇-C₂₀ aralkyl, heteroaralkyl including 6-20 atoms, C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c}; or

(vi) C₆-C₁₆ aryl or heteroaryl including 5-16 atoms, each of which is optionally substituted with from 1-10 R^{d}; or

(vii) R¹ together with R³ or R⁴ is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c}; or

(viii) R⁵ together with R³ or R⁴is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which is optionally substituted with from 1-5 R^{c};

R² is:

(i) hydrogen; or

(ii) C₁-C₂₀ alkyl or C₃-C₁₆ cycloalkyl, each of which is optionally substituted with from 1-10 R^{a}; or

(iii) C₆-C₁₆ aryl, optionally substituted with from 1-10 R^{d}; or

(iv) C₇-C₂₀ aralkyl or heteroaralkyl including 6-20 atoms, each of which is optionally substituted with 1-10 R^{c};

each of R³ and R⁴ is, independently:

(i) hydrogen or C₁-C₁₀ alkyl; or

(ii) R³ and R⁴ together are C₃-C₁₆ cycloalkyl, optionally substituted with from 1-10 R^{a}; C₃-C₁₆ halocycloalkyl; C₃-C₁₆ cycloalkenyl, heterocyclyl including 5-16 atoms, or heterocycloalkenyl including 5-16 atoms, each of which is optionally substituted with from 1-10 R^{b}; or C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c};

(iii) one of R³ or R⁴ is hydrogen or C₁-C₁₀ alkyl, and the other together with R¹ is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c};

(iv) one of R³ or R⁴is hydrogen or C₁-C₁₀ alkyl, and the other together with R⁵ is is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which can be optionally substituted with from 1-5 R^{c};

each of A and B is, independently, a bond or (CR^{e}R^{f})ₘ;

each of X and Y is, independently:

(i) hydrogen, C₁-C₆ alkyl, or hydroxy; or

(ii) X and Y together are oxo;

R^{a} at each occurrence is, independently, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});

R^{b} at each occurrence is, independently, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});

R^{c} at each occurrence is, independently, C₁-C₁₂ alkyl, C₁-C₁₂ haloalkyl, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});

R^{d} at each occurrence is, independently:

(i) halo; NR^{g}R^{h}; nitro; hydroxy; C₁-C₁₂ alkoxy; C₁-C₁₂ haloalkoxy; C₆-C₁₆ aryloxy; mercapto; C₁-C₆ thioalkoxy; C₆-C₁₆ thioaryloxy; cyano; formyl; —C(O)R^{j}, C₁-C₃ alkylenedioxy; —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NR^{m})R^{j}; S(O)ₙR^{p}; or P(O)(OR^{g})(OR^{h}); or

(ii) C₁-C₁₂ alkyl, optionally substituted with from 1-10 R^{a} and/or optionally inserted with from 1-6 heteroatoms selected from the group consisting of nitrogen, oxygen or sulfur; or

(iii) C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which is optionally substituted with from 1-10 R^{b}; or

(iv) C₁-C₁₂ haloalkyl; or

(v) C₇-C₂₀ aralkyl or heteroaralkyl including 6-20 atoms, each of which is optionally substituted with from 1-10 R^{c}; or

(vi) C₆-C₁₆ aryl or heteroaryl including 5-16 atoms, each of which is optionally substituted with (e.g., with from 1-5 of any of the following substituents or a combination thereof) C₁-C₁₂ alkyl, C₁-C₁₂ haloalkyl, halo, NR^{g}R^{h}, nitro, hydroxy, C₁-C₁₂ alkoxy, C₁-C₁₂ haloalkoxy, C₆-C₁₆ aryloxy, mercapto, C₁-C₆ thioalkoxy, C₆-C₁₆ thioaryloxy, cyano, formyl, —C(O)R^{j}, C₁-C₃ alkylenedioxy, —C(O)OR^{j}, —OC(O)R^{j}, —C(O)SR^{j}, —SC(O)R^{j}, —C(S)SR^{j}, —SC(S)R^{j}, —C(O)NR^{g}R^{h}, —NR^{k}C(O)R^{j}, —C(NR^{m})R^{j}, S(O)ₙR^{p}, or P(O)(OR^{g})(OR^{h});

each of R^{e}, R^{f}, and R^{k}, at each occurrence is, independently, hydrogen or C₁-C₁₀ alkyl;

each of R^{g}, R^{h}, and R^{j}, at each occurrence is, independently, hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; or heteroaryl including 5-16 atoms;

R^{m} is hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms; NR^{g}R^{h}, or OR^{j};

R^{p} is C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms; NR^{g}R^{h}, or OR^{j};

m is 1-20; and n is 1 or 2;

provided that when R¹ is isopropyl and X and Y together are oxo (e.g., when R¹ is isopropyl, X and Y together are oxo, and A and B are both a bond; e.g., when R¹ is isopropyl, X and Y together are oxo, A and B are both a bond, and R³ and R⁴ are both hydrogen), then R⁵ is not 4-bromophenyl, 4-benzamidophenyl, 4-methyl-phenyl, 4-isopropylphenyl, 4-isobutylphenyl, 4-t-butylphenyl, 4-methoxyphenyl, 4-isopropoxyphenyl, 4-cyclopentylphenyl, 4-cyclohexylphenyl, 4-(2-furyl)phenyl, 4-(3-furyl)phenyl, 4-(2-thienyl)phenyl, 4-(3-thienyl)phenyl, 4-(pyrrolidin-1-yl)phenyl, 4-(piperidin-1-yl)phenyl, 3-chloro-4-piperidin-1-ylphenyl, 4-(2-fluorophenyl)phenyl, 4-(3-fluorophenyl)phenyl, 4-(2-formylphenyl)phenyl, 4-(3-formylphenyl)phenyl, 4-(4-formylphenyl)phenyl, 4-(4-methylphenyl)phenyl, 4-(4-hydroxphenyl)phenyl, 4-(2-methoxyphenyl)phenyl or 4-(4-methoxyphenyl)phenyl; or a pharmaceutically acceptable salt therof.

In another aspect, this invention features a pharmaceutical composition, which includes an effective amount of a compound of formula (I): 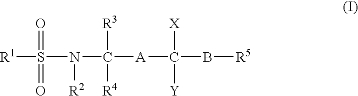
wherein:

each of R¹ and R⁵ is, independently:

(i) C₁-C₂₀ alkyl, optionally substituted with from 1-10 R^{a}; or

(ii) C₃-C₁₆ cycloalkyl, optionally substituted with from 1-10 R^{a}; or

(iii) C₁-C₂₀ haloalkyl or C₃-C₁₆ halocycloalkyl, optionally substituted with from 1-10 R^{a}; or

(iv) C₂-C₂₀ alkenyl, C₂-C₂₀ alkynyl, C₃-C₁₆ cycloalkenyl, heterocyclyl including 3-16 atoms, or heterocycloalkenyl including 3-16 atoms, each of which is optionally substituted with from 1-10 R^{b}; or

(v) C₇-C₂₀ aralkyl, heteroaralkyl including 6-20 atoms, C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c}; or

(vi) C₆-C₁₆ aryl or heteroaryl including 5-16 atoms, each of which is optionally substituted with from 1-10 R^{d}; or

(vii) R¹ together with R³ or R⁴is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c}; or

(viii) R⁵ together with R³ or R⁴ is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which is optionally substituted with from 1-5 R^{c};

R² is:

(i) hydrogen; or

(ii) C₁-C₂₀ alkyl or C₃-C₁₆ cycloalkyl, each of which is optionally substituted with from 1-10 R^{a}; or

(iii) C₆-C₁₆ aryl, optionally substituted with from 1-10 R^{d}; or

(iv) C₇-C₂₀ aralkyl or heteroaralkyl including 6-20 atoms, each of which is optionally substituted with 1-10 R^{c};

each of R³ and R⁴ is, independently:

(i) hydrogen or C₁-C₁₀ alkyl; or

(ii) R³ and R⁴ together are C₃-C₁₆ cycloalkyl, optionally substituted with from 1-10 R^{a}; C₃-C₁₆ halocycloalkyl; C₃-C₁₆ cycloalkenyl, heterocyclyl including 5-16 atoms, or heterocycloalkenyl including 5-16 atoms, each of which is optionally substituted with from 1-10 R^{b}; or C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c};

(iii) one of R³ or R⁴ is hydrogen or C₁-C₁₀ alkyl, and the other together with R¹ is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c};

(iv) one of R³ or R⁴ is hydrogen or C₁-C₁₀ alkyl, and the other together with R⁵ is is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which can be optionally substituted with from 1-5 R^{c};

each of A and B is, independently, a bond or (CR^{e}R^{f})ₘ;

each of X and Y is, independently:

(i) hydrogen, C₁-C₆ alkyl, or hydroxy; or

(ii) X and Y together are oxo;

R^{a} at each occurrence is, independently, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{h}, —SC(S)R^{j}, —C(O)NR^{g}R^{h}; NR^{k}C(O)R^{j}, —C(NR^{m})R^{j}, S(O)ₙR^{p}, or P(O)(OR^{g})(OR^{h});

R^{b} at each occurrence is, independently, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});

R^{c} at each occurrence is, independently, C₁-C₁₂ alkyl, C₁-C₁₂ haloalkyl, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});

R^{d} at each occurrence is, independently:

(i) halo; NR^{g}R^{h}; nitro; hydroxy; C₁-C₁₂ alkoxy; C₁-C₁₂ haloalkoxy; C₆-C₁₆ aryloxy; mercapto; C₁-C₆ thioalkoxy; C₆-C₁₆ thioaryloxy; cyano; formyl; —C(O)R^{j}, C₁-C₃ alkylenedioxy; —C(O)OR^{j}; —OC(O)R^{j}; —C(O)SR^{j}; —SC(O)R^{j}; —C(S)SR^{j}; —SC(S)R^{j}; —C(O)NR^{g}R^{h}; NR^{k}C(O)R^{j}; —C(NR^{m})R^{j}; S(O)ₙR^{p}; or P(O)(OR^{g})(OR^{h}); or

(ii) C₁-C₁₂ alkyl, optionally substituted with from 1-10 R^{a} and/or optionally inserted with from 1-6 heteroatoms selected from the group consisting of nitrogen, oxygen or sulfur; or

(iii) C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which is optionally substituted with from 1-10 R^{b}; or

(iv) C₁-C₁₂ haloalkyl; or

(v) C₇-C₂₀ aralkyl or heteroaralkyl including 6-20 atoms, each of which is optionally substituted with from 1-10 R^{c}; or

(vi) C₆-C₁₆ aryl or heteroaryl including 5-16 atoms, each of which is optionally substituted with (e.g., with from 1-5 of any of the following substituents or a combination thereof) C₁-C₁₂ alkyl, C₁-C₁₂ haloalkyl, halo, NR^{g}R^{h}, nitro, hydroxy, C₁-C₁₂ alkoxy, C₁-C₁₂ haloalkoxy, C₆-C₁₆ aryloxy, mercapto, C₁-C₆ thioalkoxy, C₆-C₁₆ thioaryloxy, cyano, formyl, —C(O)R^{j}, C₁-C₃ alkylenedioxy, —C(O)OR^{j}, —OC(O)R^{j}, —C(O)SR^{j}, —SC(O)R^{j}, —C(S)SR^{j}, —SC(S)R^{j}, —C(O)NR^{g}R^{h}, —NR^{k}C(O)R^{j}, —C(NR^{m})R^{j}, S(O)ₙR^{p}, or P(O)(OR^{g})(OR^{h});

each of R^{e}, R^{f}, and R^{k}, at each occurrence is, independently, hydrogen or C₁-C₁₀ alkyl;

each of R^{g}, R^{h}, and R^{j}, at each occurrence is, independently, hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; or heteroaryl including 5-16 atoms;

R^{m} is hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms; NR^{g}R^{h}, or OR^{j};

R^{p} is C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms;NR^{g}R^{h}, or OR^{j};

m is 1-20; and n is 1 or 2;

provided that when RI is isopropyl and X and Y together are oxo (e.g., when R¹ is isopropyl, X and Y together are oxo, and A and B are both a bond; e.g., when R¹ is isopropyl, X and Y together are oxo, A and B are both a bond, and R³ and R⁴ are both hydrogen), then R⁵ is not 4-bromophenyl, 4-benzamidophenyl, 4-methyl-phenyl, 4-isopropylphenyl, 4-isobutylphenyl, 4-t-butylphenyl, 4-methoxyphenyl, 4-isopropoxyphenyl, 4-cyclopentylphenyl, 4-cyclohexylphenyl, 4-(2-furyl)phenyl, 4-(3-furyl)phenyl, 4-(2-thienyl)phenyl, 4-(3-thienyl)phenyl, 4-(pyrrolidin-1-yl)phenyl, 4-(piperidin-1-yl)phenyl, 3-chloro-4-piperidin-1-ylphenyl, 4-(2-fluorophenyl)phenyl, 4-(3-fluorophenyl)phenyl, 4-(2-formylphenyl)phenyl, 4-(3-formylphenyl)phenyl, 4-(4-formylphenyl)phenyl, 4-(4-methylphenyl)phenyl, 4-(4-hydroxphenyl)phenyl, 4-(2-methoxyphenyl)phenyl or 4-(4-methoxyphenyl)phenyl; or a pharmaceutically acceptable salt therof;

and a pharmaceutically acceptable carrier.

In a further aspect, this invention relates to a method for treating a disease or condition mediated by excess or uncontrolled amounts of cortisol and/or other corticosteroids, which includes administering to a subject in need thereof an effective amount of a compound of formula (I):

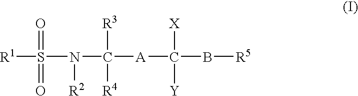

wherein:

each of R¹ and R⁵ can be, independently:

(i) C₁-C₂₀ alkyl (e.g., methyl, ethyl, propyl, isopropyl), optionally substituted with from 1-10 R^{a} and/or optionally inserted with from 1-10 heteroatoms selected from the group consisting of nitrogen, oxygen or sulfur; or

(ii) C₃-C₁₆ cycloalkyl, optionally substituted with from 1-10 R^{a}; or

(iii) C₁-C₂₀ haloalkyl or C₃-C₁₆ halocycloalkyl, each of which can be optionally substituted with from 1-10 R^{a}; or

(iv) C₂-C₂₀ alkenyl, C₂-C₂₀ alkynyl, C₃-C₁₆ cycloalkenyl, heterocyclyl including 3-16 atoms, or heterocycloalkenyl including 3-16 atoms, each of which can be optionally substituted with from 1-10 R^{b}; or

(v) C₇-C₂₀ aralkyl (e.g., benzyl, 4′-chlorobenzyl, 2-phenylethyl), heteroaralkyl including 6-20 atoms, C₈-C₂₀ arylcycloalkyl, C₈-C₂₀ arylcycloalkenyl, arylheterocyclyl including 8-20 atoms, or arylheterocycloalkenyl including 8-20 atoms, each of which can be optionally substituted with from 1-10 (e.g., 1-2) R^{c}; or

(vi) C₆-C₁₆ aryl (e.g., C₆-C₁₀ aryl, naphthyl, phenyl, 4-tert-butylphenyl, 4-biphenyl, 4-chlorophenyl, 3,5-dimethylphenyl, 4-bromophenyl, 2-fluorophenyl, 4-trifluoromethylphenyl, 4-methoxyphenyl) or heteroaryl (e.g., thienyl, benzothienyl, furyl, imidazolyl, isoxazolyl) including 5-16 atoms, each of which can be optionally substituted with from 1-10 (e.g., 1-2, 1-3) R^{d}; or

(vii) R¹ together with R³ or R⁴ can be heterocyclyl including 3-10 atoms or heterocycloalkenyl including 5-10 atoms, each of which can be optionally substituted with from 1-5 R^{b}; or arylheterocyclyl including 8-12 atoms (e.g., 9-12 atoms) or arylheterocycloalkenyl including 8-12 atoms, each of which can be optionally substituted with from 1-5 R^{c}; or

(viii) R⁵ together with R³ or R⁴ can be C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which can be optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which can be optionally substituted with from 1-5 R^{c};

R² can be:

(i) hydrogen; or

(ii) C₁-C₂₀ alkyl (e.g., methyl or ethyl) or C₃-C₁₆ cycloalkyl, each of which can be optionally substituted with from 1-10 R^{a}; or

(iii) C₆-C₁₆ aryl, optionally substituted with from 1-10 R^{d}; or

(iv) C₇-C₂₀ aralkyl (e.g., benzyl, 2-phenylethyl) or heteroaralkyl including 6-20 atoms, each of which can be optionally substituted with 1-10 R^{c} (e.g., oxo);

each of R³ and R⁴ can be, independently:

(i) hydrogen or C₁-C₁₀ alkyl (e.g., C₁-C₆ alkyl; H, H; CH₃, CH₃; H, CH₃; H, isopropyl); or

(ii) R³ and R⁴ together can be C₃-C₁₆ cycloalkyl, optionally substituted with from 1-10 R^{a}; C₃-C₁₆ halocycloalkyl; C₃-C₁₆ cycloalkenyl, heterocyclyl including 5-16 atoms, or heterocycloalkenyl including 5-16 atoms, each of which can be optionally substituted with from 1-10 R^{b}; or C₈-C₂₀ arylcycloalkyl, C₈-C₂₀ arylcycloalkenyl, arylheterocyclyl including 8-20 atoms, or arylheterocycloalkenyl including 8-20 atoms, each of which can be optionally substituted with from 1-10 R^{c};

(iii) one of R³ or R⁴ can be hydrogen or C₁-C₁₀ alkyl, and the other together with R¹ can be heterocyclyl including 3-10 atoms or heterocycloalkenyl including 5-10 atoms, each of which can be optionally substituted with from 1-5 R^{b}; or arylheterocyclyl including 8-12 (e.g., 9-12 atoms) atoms or arylheterocycloalkenyl including 8-12 atoms, each of which can be optionally substituted with from 1-5 R^{c}.

(iv) one of R³ or R⁴ can be hydrogen or C₁-C₁₀ alkyl, and the other together with R⁵ can be C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which can be optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which can be optionally substituted with from 1-5 R^{c};

each of A and B can be, independently, a bond or (CR^{e}R^{f})ₘ;

each of X and Y can be, independently:

(i) hydrogen, C₁-C₆ alkyl, or hydroxy; or

(ii) X and Y together can be oxo;

R^{a} at each occurrence can be, independently, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});

R^{b} at each occurrence can be, independently, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});

R^{c} at each occurrence can be, independently, C₁-C₁₂ alkyl, C₁-C₁₂ haloalkyl, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});

R^{d} at each occurrence can be, independently:

(i) halo; NR^{g}R^{h}; nitro; hydroxy; C₁-C₁₂ alkoxy; C₁-C₁₂ haloalkoxy; C₆-C₁₆ aryloxy; mercapto; C₁-C₆ thioalkoxy; C₆-C₁₆ thioaryloxy; cyano; formyl; —C(O)R^{j}, C₁-C₃ alkylenedioxy; —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NR^{m})R^{j}; S(O)ₙR^{p}; or P(O)(OR^{g})(OR^{h}); or

(ii) C₁-C₁₂ alkyl, optionally substituted with from 1-10 R^{a} and/or optionally inserted with from 1-6 heteroatoms selected from the group consisting of nitrogen, oxygen or sulfur; or

(iii) C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which can be optionally substituted with from 1-10 R^{b}; or

(iv) C₁-C₁₂ haloalkyl; or

(v) C₇-C₂₀ aralkyl or heteroaralkyl including 6-20 atoms, each of which can be optionally substituted with from 1-10 R^{c}; or

(vi) C₆-C₁₆ aryl or heteroaryl including 5-16 atoms, each of which can be optionally substituted with (e.g., with from 1-5 of any of the following substituents or a combination thereof) C₁-C₁₂ alkyl, C₁-C₁₂ haloalkyl, halo, NR^{g}R^{h}, nitro, hydroxy, C₁-C₁₂ alkoxy, C₁-C₁₂ haloalkoxy, C₆-C₁₆ aryloxy, mercapto, C₁-C₆ thioalkoxy, C₆-C₁₆ thioaryloxy, cyano, formyl, —C(O)R^{j}, C₁-C₃ alkylenedioxy, —C(O)OR^{j}, —OC(O)R^{j}, —C(O)SR^{j}, —SC(O)R^{j}, —C(S)SR^{j}, —SC(S)R^{j}, —C(O)NR^{g}R^{h}, —NR^{k}C(O)R^{j}, —C(NR^{m})R^{j}, S(O)ₙR^{p}, or P(O)(OR^{g})(OR^{h});

each of R^{e}, R^{f}, and R^{k}, at each occurrence can be, independently, hydrogen or C₁-C₁₀ alkyl;

each of R^{g}, R^{h}, and R^{j}, at each occurrence can be, independently, hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; or heteroaryl including 5-16 atoms;

R^{m} can be hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms; NR^{g}R^{h}, or OR^{j};

R^{p} can be C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms;NR^{g}R^{h}, or OR^{j};

m can be 1-20; and

n can be 1 or 2; or a pharmaceutically acceptable salt therof.

In one aspect of the invention, this invention relates to methods for treating diabetes (e.g., type I diabetes, type 2 diabetes), which includes administering to a subject in need thereof an effective amount of a compound of formula I (e.g., a compound having formula (I), (II), (III), or (IV), e.g., any of the compounds described herein) or a pharmaceutically acceptable salt thereof.

In another aspect of the invention, this invention relates to methods for treating Syndrome X, which includes administering to a subject in need thereof an effective amount of a compound of formula I (e.g., a compound having formula (I), (II), (III), or (IV), e.g., any of the compounds described herein) or a pharmaceutically acceptable salt thereof.

In a further aspect of the invention, this invention relates to methods for treating hyperglycemia, diabetes or insulin resistance, which includes administering to a subject in need thereof an effective amount of a compound of formula I (e.g., a compound having formula (I), (II), (III), or (IV), e.g., any of the compounds described herein) or a pharmaceutically acceptable salt thereof.

In one aspect of the invention, this invention relates to methods for treating obesity, which includes administering to a subject in need thereof an effective amount of a compound of formula I (e.g., a compound having formula (I), (II), (III), or (IV), e.g., any of the compounds described herein) or a pharmaceutically acceptable salt thereof.

In another aspect of the invention, this invention relates to methods for treating a lipid disorder selected from the group consisting of dyslipidemia, hyperlipidemia, hypertriglyceridemia, hypercholesterolemia, low HDL and high LDL, which includes administering to a subject in need thereof an effective amount of a compound of formula I (e.g., a compound having formula (I), (II), (III), or (IV), e.g., any of the compounds described herein) or a pharmaceutically acceptable salt thereof.

In a further aspect of the invention, this invention relates to methods for treating atherosclerosis, which include administering to a subject in need thereof an effective amount of a compound of formula I (e.g., a compound having formula (I), (II), (III), or (IV), e.g., any of the compounds described herein) or a pharmaceutically acceptable salt thereof.

In one aspect of the invention, this invention relates to methods for treating a cognitive disorder (e.g., Alzheimer's disease), which includes administering to a subject in need thereof an effective amount of a compound of formula I (e.g., a compound having formula (I), (II), (III), or (IV), e.g., any of the compounds described herein) or a pharmaceutically acceptable salt thereof.

In another aspect of the invention, this invention relates to methods for promoting wound healing, which includes administering to a subject in need thereof an effective amount of a compound of formula I (e.g., a compound having formula (I), (II), (III), or (IV), e.g., any of the compounds described herein) or a pharmaceutically acceptable salt thereof.

In a further aspect aspect of the invention, this invention relates to methods for treating, controlling, ameliorating, preventing, delaying the onset of, or reducing the risk of developing one or more of diabetes (e.g., type 1 or type 2 diabetes), Syndrome X, hyperglycemia, low glucose tolerance, insulin resistance, obesity, lipid disorders, dyslipidemia, hyperlipidemia, hypertriglyceridemia, hypercholesterolemia, low HDL levels, high LDL levels, atherosclerosis and its sequelae, vascular restenosis, pancreatitis, abdominal obesity, neurodegenerative disease, retinopathy, nephropathy, neuropathy, hypertension, coronary heart disease, stroke, peripheral vascular disease, Cushing's syndrome, glaucoma, osteoperosis, hyperinsulinemia, tuberculosis, psoriasis, cognitive disorders and dementia (e.g., impairment associated with aging and of neuronal dysfunction, e.g., Alzheimer's disease), depression, viral diseases, inflammatory disorders, immune disorders); or promoting wound healing, which includes administering to a subject in need thereof an effective amount of a compound of formula I (e.g., a compound having formula (I), (II), (III), or (IV), e.g., any of the compounds described herein) or a pharmaceutically acceptable salt thereof.

The invention also relates generally to inhibiting 11-beta HSD1 with the sulfonamide compounds. In some embodiments, the methods can include, e.g., contacting an 11βHSD1 in a sample (e.g., a tissue) with a compound having formula (I) (e.g., a compound having formula (I), (II), (III), or (IV), e.g., any of the compounds described herein). In other embodiments, the methods can include administering a compound having formula (I) to a subject (e.g., a mammal, e.g., a mammal subject to or at risk for diseases mediated by abnormally high levels of cortisol and other 11β-hydroxysteroids, e.g., NIDDM, obesity, dyslipidemia, syndrome X, and hypertension). Accordingly, in yet another aspect, this invention includes methods of screening for compounds that inhibit 11βHSD1.

In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In a further aspect, this invention also relates to methods of making compounds described herein. Alternatively, the method includes taking any one of the intermediate compounds described herein and reacting it with one or more chemical reagents in one or more steps to produce a compound described herein.

In one aspect, this invention relates to a packaged product. The packaged product includes a container, one of the aforementioned compounds in the container, and a legend (e.g., a label or an insert) associated with the container and indicating administration of the compound for treatment and control of diseases mediated by abnormally high levels of cortisol and other 11β-hydroxysteroids, e.g., NIDDM and Syndrome X.

In another aspect, the invention relates to a compound (including a pharmaceutically acceptable salt thereof) of any of the formulae delineated herein, or a composition comprising a compound (including a pharmaceutically acceptable salt thereof) of any of the formulae delineated herein. In some embodiments, the composition can further include a pharmaceutically acceptable adjuvant, carrier or diluent and/or an additional therapeutic agent.

Embodiments can include one or more of the following features.

The compound can be a compound of formula (II): 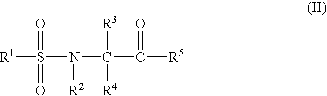
in which R¹, R², R³, R⁴, and R⁵ are as defined herein, or a pharmaceutically acceptable salt thereof.

In some embodiments, when R^{c} is attached to an aryl or heteroaryl moiety, R^{c} can further include as permissible substituents: C₁-C₁₂ alkyl substituted with from 1-10 R^{a} and/or optionally inserted with from 1-6 heteroatoms selected from the group consisting of nitrogen, oxygen or sulfur; or C₁-C₁₂ haloalkyl substituted with from 1-10 R^{a}; or C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which can be optionally substituted with from 1-10 R^{b}; or C₇-C₂₀ aralkyl or heteroaralkyl including 6-20 atoms, each of which can be optionally substituted with from 1-10 R^{c}.

In some embodiments, when R is a substituted C₆-C₁₆ aryl or heteroaryl including 5-16 atoms, then the permissible substitutents for the substituted C₆-C₁₆ aryl or heteroaryl including 5-16 atoms can further include: C₁-C₁₂ alkyl substituted with from 1-10 R^{a} and/or optionally inserted with from 1-6 heteroatoms selected from the group consisting of nitrogen, oxygen or sulfur; or C₁-C₁₂ haloalkyl substituted with from 1-10 R^{a}; or C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which can be optionally substituted with from 1-10 R^{b}; or C₇-C₂₀ aralkyl or heteroaralkyl including 6-20 atoms, each of which can be optionally substituted with from 1-10 R^{c}.

In some embodiments, it is provided that when R¹ is isopropyl and X and Y together are oxo (e.g., when R¹ is isopropyl, X and Y together are oxo, and A and B are both a bond; e.g., when R¹ is isopropyl, X and Y together are oxo, A and B are both a bond, and R³ and R⁴ are both hydrogen), then R⁵ is not 4-bromophenyl, 4-benzamidophenyl, 4-methyl-phenyl, 4-isopropylphenyl, 4-isobutylphenyl, 4-t-butylphenyl, 4-methoxyphenyl, 4-isopropoxyphenyl, 4-cyclopentylphenyl, 4-cyclohexylphenyl, 4-(2-hydroxy-methylphenyl)phenyl, 4-(4-hydroxymethylphenyl)phenyl, 4-(2-furyl)phenyl, 4-(3-furyl)phenyl, 4-(2-thienyl)phenyl, 4-(3-thienyl)phenyl, 4-(pyrrolidin-1-yl)phenyl, 4-(piperidin-1-yl)phenyl, 3-chloro-4-piperidin-1-ylphenyl, 4-(2-fluorophenyl)phenyl, 4-(3-fluorophenyl)phenyl, 4-(2-formylphenyl)phenyl, 4-(3-formylphenyl)phenyl, 4-(4-formylphenyl)phenyl, 4-(4-methylphenyl)phenyl, 4-(4-hydroxphenyl)phenyl, 4-(2-methoxyphenyl)phenyl or 4-(4-methoxyphenyl)phenyl.

R¹ can be C₆-C₁₆ aryl, optionally substituted with from 1-10 R^{d}; heteroaryl including 5-16 atoms, optionally substituted with from 1-10 R^{d}; C₇-C₂₀ aralkyl, optionally substituted with from 1-10 R^{c}; or C₁-C₂₀ alkyl.

R¹ can be C₆-C₁₀ aryl, optionally substituted with from 1-3 R^{d}.

R¹ can be naphthyl.

R¹ can be phenyl, optionally substituted with from 1-3 (e.g., 1-2, 1) R^{d}. R^{d}, at each occurrence, can be, independently, C₁-C₆ alkyl, C₆-C₁₀ aryl, C₁-C₆ alkoxy, halo, C₆-C₁₀ aryloxy, or nitro. For example, R¹ can be phenyl, 4-tert-butylphenyl, 4-biphenyl, 4-chlorophenyl, 3,5-dimethylphenyl, 4-bromophenyl, or 2-fluorophenyl.

R¹ can be heteroaryl including 5-10 atoms, optionally substituted with from 1-2 R^{d}. For example, R¹ can be thienyl, furyl, imidazolyl, or isoxazolyl, optionally substituted with from 1-2 R^{d}. R^{d}, at each occurrence, can be, independently, C₁-C₆ alkyl, C₁-C₆ haloalkyl, or halo.

R¹ can be C₇-C₁₀ aralkyl, optionally substituted with from 1-2 R^{c}. For example, R¹ can be benzyl or 2-phenylethyl, optionally substituted with halo.

R¹ can be C₁-C₁₂ alkyl. For example, R¹ can be methyl, ethyl, propyl, or isopropyl. In certain embodiments, R¹ can be methyl, ethyl, or propyl. In other embodiments, R¹ can be isopropyl.

R⁵ can be C₆-C₁₆ aryl, optionally substituted with from 1-10 R^{d}; or heteroaryl including 5-16 atoms, optionally substituted with from 1-10 R^{d}.

R⁵ can be C₆-C₁₀ aryl, optionally substituted with from 1-2 R^{d}. For example, R⁵ can be naphthyl. R⁵ can also be phenyl, 4-biphenyl, 4-trifluoromethylphenyl, or 4-methoxyphenyl. In certain embodiments, R⁵ can be phenyl, 4-biphenyl, or 4-trifluoromethylphenyl. In other embodiments, R⁵ can be 4-methoxyphenyl.

R⁵ is heteroaryl including 5-10 atoms, optionally substituted with from 1-2 R^{d}. For example, R⁵ can be thienyl, benzothienyl, furyl, imidazolyl, or isoxazolyl, optionally substituted with from 1-2 R^{d}. R^{d}, at each occurrence, can be, independently, C₁-C₆ alkyl, C₁-C₆ haloalkyl, or halo.

R² can be hydrogen.

R can be C₁-C₂₀ alkyl or C₃-C₁₆ cycloalkyl, each of which is optionally substituted with from 1-10 R^{a}; C₆-C₁₆ aryl, optionally substituted with from 1-10 R^{d}; or C₇-C₂₀ aralkyl or heteroaralkyl including 6-20 atoms, each of which is optionally substituted with 1-5 R^{c}.

R² can be methyl or ethyl.

R² can be C₇-C₁₀ aralkyl, optionally substituted with oxo or C₁-C₄ haloalkyl.

Each of A and B can be a bond.

X and Y together can be oxo.

Each of R³ and R⁴ can be, independently hydrogen or C₁-C₁₀ alkyl. For example, each of R³ and R⁴ can be hydrogen; or each of R³ and R⁴ can be C₁-C₆ alkyl (e.g., each of R³ and R⁴ can be methyl; one of R³ or R⁴ can be hydrogen, and the other can be C₁-C₆ alkyl (e.g., one of R³ or R⁴ can be hydrogen, and the other can be methyl or isopropyl).

One of R³ or R⁴ can be hydrogen or C₁-C₁₀ alkyl, and the other together with R¹ can be heterocyclyl including 3-10 atoms or heterocycloalkenyl including 5-10 atoms, each of which can be optionally substituted with from 1-5 R^{b}; or arylheterocyclyl including 8-12 atoms or arylheterocycloalkenyl including 8-12 atoms, each of which can be optionally substituted with from 1-5 R^{c}. For example, one of R³ or R⁴ can be hydrogen, and the other together with R¹ can be arylheterocyclyl including 9-12 atoms.

One of R³ or R⁴ can be hydrogen or C₁-C₁₀ alkyl, and the other together with R⁵ can be C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which is optionally substituted with from 1-5 R^{c}.

One of R³ or R⁴ can be hydrogen, and the other together with R¹ can be C₈-C₁₂ arylcycloalkyl.

Each of R³ and R⁴ can be, independently hydrogen or C₁-C₁₀ alkyl, each of A and B can be a bond, and X and Y together can be oxo.

R¹ can be phenyl, optionally substituted with from 1-2 R^{d}. R^{d}, at each occurrence, can be, independently, C₁-C₆ alkyl, C₆-C₁₀ aryl, C₁-C₆ alkoxy, halo, C₆-C₁₀ aryloxy, cyano, or nitro. For example, R¹ can be phenyl, 4-tert-butylphenyl, 4-biphenyl, 4-chlorophenyl, 3,5-dimethylphenyl, 4-bromophenyl, or 2-fluorophenyl.

R¹ can be heteroaryl including 5-10 atoms, optionally substituted with from 1-2 R^{d}. For example, R¹ can be thienyl, furyl, imidazolyl, or isoxazolyl, optionally substituted with from 1-2 R^{d}. R^{d}, at each occurrence can be, independently, C₁-C₆ alkyl, C₁-C₆ haloalkyl, or halo.

In some embodiments, R¹ cannot be C₁-C₁₀ alkyl, optionally substituted with from 1-10 R^{a} and/or optionally inserted with nitrogen, oxygen or sulfur; C₁-C₁₀ haloalkyl; C₃-C₁₆ cycloalkyl, optionally substituted with from 1-10 R^{a}; C₂-C₁₀ alkenyl; C₂-C₁₀ alkynyl; C₃-C₁₆ cycloalkenyl; heterocyclyl including 3-6 atoms, or heterocycloalkenyl including 3-6 atoms, C₇-C₂₀ aralkyl or heteroaralkyl including 6-20 atoms, each of which is optionally substituted with from 1-10 R^{c}; C₆ aryl, optionally substituted with 1-2 R^{d}; or heteroaryl including 5 atoms, optionally substituted with 1-2 R^{d}.

The compound can be N-(2-Oxo-2-phenyl-ethyl)-benzenesulfonamide, 3-Chloro-2-methyl-N-(2-oxo-2-phenyl-ethyl)-benzenesulfonamide, 3-Methyl-N-(2-oxo-2-phenyl-ethyl)-benzenesulfonamide, Biphenyl-4-sulfonic acid (2-oxo-2-phenyl-ethyl)-amide, 4-Bromo-N-(2-oxo-2-phenyl-ethyl)-benzenesulfonamide, 2-Phenyl-ethanesulfonic acid (2-oxo-2-phenyl-ethyl)-amide, 4-Chloro-N-(2-oxo-2-phenyl-ethyl)-benzenesulfonamide, 2-Fluoro-N-(2-oxo-2-phenyl-ethyl)-benzenesulfonamide, 2,5-Dichloro-thiophene-3-sulfonic acid (2-oxo-2-phenyl-ethyl)-amide, 5-Chloro-thiophene-2-sulfonic acid (2-oxo-2-phenyl-ethyl)-amide, 3,4-Dimethoxy-N-(2-oxo-2-phenyl-ethyl)-benzenesulfonamide, 3,5-Dimethyl-N-(2-oxo-2-phenyl-ethyl)-benzenesulfonamide, 3-Cyano-4-fluoro-N-(2-oxo-2-phenyl-ethyl)-benzenesulfonamide, 4-tert-Butyl-N-(2-oxo-2-phenyl-ethyl)-benzenesulfonamide, 1-Methyl-1H-imidazole-4-sulfonic acid (2-oxo-2-phenyl-ethyl)-amide, Naphthalene-2-sulfonic acid (2-oxo-2-phenyl-ethyl)-amide, N-(2-Oxo-2-phenyl-ethyl)-4-phenoxy-benzenesulfonamide, Biphenyl-3-sulfonic acid (2-oxo-2-phenyl-ethyl)-amide, C-(4-Chloro-phenyl)-N-(2-oxo-2-phenyl-ethyl)-methanesulfonamide, C-(3-Nitro-phenyl)-N-(2-oxo-2-phenyl-ethyl)-methanesulfonamide, C-(3,5-Dichloro-phenyl)-N-(2-oxo-2-phenyl-ethyl)-methanesulfonamide, C-(3-Chloro-phenyl)-N-(2-oxo-2-phenyl-ethyl)-methanesulfonamide, Propane-1-sulfonic acid (2-oxo-2-phenyl-ethyl)-amide, Ethanesulfonic acid (2-oxo-2-phenyl-ethyl)-amide, Propane-2-sulfonic acid (2-oxo-2-phenyl-ethyl)-amide, N-(2-Oxo-2-phenyl-ethyl)-C-phenyl-methanesulfonamide, 4-tert-Butyl-N-[2-(4-methoxy-phenyl)-2-oxo-ethyl]-benzenesulfonamide, N-[2-(4-Methoxy-phenyl)-2-oxo-ethyl]-4-propyl-benzenesulfonamide, 4-Ethyl-N-[2-(4-methoxy-phenyl)-2-oxo-ethyl]-benzenesulfonamide, 1-Methyl-1H-imidazole-4-sulfonic acid [2-(4-methoxy-phenyl)-2-oxo-ethyl]-amide, 3,5-Dimethyl-isoxazole-4-sulfonic acid [2-(4-methoxy-phenyl)-2-oxo-ethyl]-amide, 3,5-Dimethyl-N-(2-naphthalen-2-yl-2-oxo-ethyl)-benzenesulfonamide, N-(2-Naphthalen-2-yl-2-oxo-ethyl)-benzenesulfonamide, Naphthalene-2-sulfonic acid (2-naphthalen-2-yl-2-oxo-ethyl)-amide, 2-Fluoro-N-(2-naphthalen-2-yl-2-oxo-ethyl)-benzenesulfonamide, 4-Chloro-N-(2-naphthalen-2-yl-2-oxo-ethyl)-benzenesulfonamide, N-(2-Naphthalen-2-yl-2-oxo-ethyl)-4-phenoxy-benzenesulfonamide, 3,4-Dimethoxy-N-(2-naphthalen-2-yl-2-oxo-ethyl)-benzenesulfonamide, 5-Chloro-thiophene-2-sulfonic acid (2-naphthalen-2-yl-2-oxo-ethyl)-amide, 4-Bromo-N-(2-naphthalen-2-yl-2-oxo-ethyl)-benzenesulfonamide, N-[2-Oxo-2-(4-trifluoromethyl-phenyl)-ethyl]-benzenesulfonamide, Propane-1-sulfonic acid [2-oxo-2-(4-trifluoromethyl-phenyl)-ethyl]-amide, Biphenyl-4-sulfonic acid [2-oxo-2-(4-trifluoromethyl-phenyl)-ethyl]-amide, N-[2-Oxo-2-(4-trifluoromethyl-phenyl)-ethyl]-C-phenyl-methanesulfonamide, N-(1,1-Dimethyl-2-oxo-2-phenyl-ethyl)-benzenesulfonamide, Naphthalene-2-sulfonic acid (1,1-dimethyl-2-oxo-2-phenyl-ethyl)-amide, 4-tert-Butyl-N-(1,1-dimethyl-2-oxo-2-phenyl-ethyl)-benzenesulfonamide, Propane-1-sulfonic acid (1,1-dimethyl-2-oxo-2-phenyl-ethyl)-amide, Biphenyl-4-sulfonic acid (1,1-dimethyl-2-oxo-2-phenyl-ethyl)-amide, 4-Chloro-N-(1,1-dimethyl-2-oxo-2-phenyl-ethyl)-benzenesulfonamide, N-(1,1-Dimethyl-2-oxo-2-phenyl-ethyl)-4-methyl-benzenesulfonamide, N-(1-Methyl-2-oxo-2-phenyl-ethyl)-benzenesulfonamide, N-(1-Benzoyl-2-methyl-propyl)-benzenesulfonamide, 4-tert-Butyl-N-[2-(4-methoxy-phenyl)-2-oxo-ethyl]-N-methyl-benzenesulfonamide, N-Methyl-N-(2-oxo-2-phenyl-ethyl)-benzenesulfonamide, N-Benzyl-N-(2-oxo-2-phenyl-ethyl)-benzenesulfonamide, N-(4-Chloro-benzyl)-N-(2-oxo-2-phenyl-ethyl)-benzenesulfonamide, N-(2-Oxo-2-phenyl-ethyl)-N-(4-trifluoromethyl-benzyl)-benzenesulfonamide, Propane-1-sulfonic acid ethyl-(1-methyl-2-oxo-2-phenyl-ethyl)-amide, N,N-Bis-(2-oxo-2-phenyl-ethyl)-benzenesulfonamide, N-Ethyl-N-(1-methyl-2-oxo-2-phenyl-ethyl)-benzenesulfonamide, N-Ethyl-N-(1-methyl-2-oxo-2-phenyl-ethyl)-C-phenyl-methanesulfonamide, 4-Chloro-N-ethyl-N-(1-methyl-2-oxo-2-phenyl-ethyl)-benzenesulfonamide, 1-Methyl-1H-imidazole-4-sulfonic acid ethyl-(1-methyl-2-oxo-2-phenyl-ethyl)-amide, N-benzyl-4-tert-butyl-N-(2-oxo-2-phenylethyl)benzenesulfonamide, N-benzyl-4-tert-butyl-N-[2-oxo-2-(3-thienyl)ethyl]benzenesulfonamide, N-[2-(1-benzothien-2-yl)-2-oxoethyl]-N-benzyl-4-tert-butylbenzenesulfonamide, N-(1,1-dimethyl-2-oxo-2-phenylethyl)naphthalene-2-sulfonamide, 4-tert-butyl-N-(1,1-dimethyl-2-oxo-2-phenylethyl)benzenesulfonamide, N-(1,1-dimethyl-2-oxo-2-phenylethyl)-4-propylbenzenesulfonamide, N-(1,1-dimethyl-2-oxo-2-phenylethyl)biphenyl-4-sulfonamide, N-(1,1-dimethyl-2-oxo-2-phenylethyl)-4-methylbenzenesulfonamide, 4-chloro-N-(1,1-dimethyl-2-oxo-2-phenylethyl)benzenesulfonamide, N-(1,1-dimethyl-2-oxo-2-phenylethyl)-1-methyl-1H-imidazole-4-sulfonamide, 3-chloro-N-(1,1-dimethyl-2-oxo-2-phenylethyl)-2-methylbenzenesulfonamide, N-(1,1-dimethyl-2-oxo-2-phenylethyl)-2-fluorobenzenesulfonamide, N-(1,1-dimethyl-2-oxo-2-phenylethyl)-3,5-dimethylbenzenesulfonamide, N-(1,1-dimethyl-2-oxo-2-phenylethyl)biphenyl-3-sulfonamide, N-(1,1-dimethyl-2-oxo-2-phenylethyl)-1-phenylmethanesulfonamide, N-(1,1-dimethyl-2-oxo-2-phenylethyl)-4-phenoxybenzenesulfonamide, 3-chloro-N-(1,1-dimethyl-2-oxo-2-phenylethyl)-5-fluoro-2-methylbenzenesulfonamide, 4-bromo-N-(1,1-dimethyl-2-oxo-2-phenylethyl)benzenesulfonamide, 1-(3,5-dichlorophenyl)-N-(1,1-dimethyl-2-oxo-2-phenylethyl)methanesulfonamide, 1-(4-chlorophenyl)-N-(1,1-dimethyl-2-oxo-2-phenylethyl)methanesulfonamide, 4-chloro-N-methyl-N-{2-oxo-2-[4-(trifluoromethyl)phenyl]ethyl}benzenesulfonamide, 4-chloro-N-methyl-N-(2-oxo-2-phenylethyl)benzenesulfonamide, N-(1,1-dimethyl-2-oxo-2-phenylethyl)-3-fluoro-4-methylbenzenesulfonamide, N-(1,1-dimethyl-2-oxo-2-phenylethyl)-5-methyl-2-(trifluoromethyl)furan-3-sulfonamide, 4-chloro-N-methyl-N-[2-oxo-2-(3-thienyl)ethyl]benzenesulfonamide, N-(2-biphenyl-4-yl-2-oxoethyl)-4-chloro-N-methylbenzenesulfonamide, N-[2-(4-bromophenyl)-2-oxoethyl]-4-chloro-N-methylbenzenesulfonamide, 4-tert-butyl-N-ethyl-N-(1-methyl-2-oxo-2-phenylethyl)benzenesulfonamide, 4-bromo-N-ethyl-N-(1-methyl-2-oxo-2-phenylethyl)benzenesulfonamide, N-ethyl-N-(1-methyl-2-oxo-2-phenylethyl)naphthalene-2-sulfonamide, N-ethyl-2-fluoro-N-(1-methyl-2-oxo-2-phenylethyl)benzenesulfonamide, N-ethyl-N-(1-methyl-2-oxo-2-phenylethyl)biphenyl-4-sulfonamide, N-ethyl-N-(1-methyl-2-oxo-2-phenylethyl)-4-propylbenzenesulfonamide, N-ethyl-4-methyl-N-(1-methyl-2-oxo-2-phenylethyl)benzenesulfonamide, N-ethyl-3,5-dimethyl-N-(1-methyl-2-oxo-2-phenylethyl)benzenesulfonamide, 3-chloro-N-ethyl-2-methyl-N-(1-methyl-2-oxo-2-phenylethyl)benzenesulfonamide, N-ethyl-N-(1-methyl-2-oxo-2-phenylethyl)biphenyl-3-sulfonamide, N-(4-chlorobenzyl)-N-(2-oxo-2-phenylethyl)benzenesulfonamide, N-(2-oxo-2-phenylethyl)-N-(4-(trifluoromethyl)benzyl)benzenesulfonamide, N-(2-(4-methoxyphenyl)-2-oxoethyl)propane-1-sulfonamide, N-(2-(4-methoxyphenyl)-2-oxoethyl)ethanesulfonamide, N-ethyl-N-(1-oxo-1-phenylpropan-2-yl)benzenesulfonamide, N-(2-(biphenyl-4-yl)-2-oxoethyl)benzenesulfonamide, N-(2-oxo-2-(4-(trifluoromethyl)phenyl)ethyl)benzenesulfonamide, or N-(2-methyl-1-oxo-1-phenylpropan-2-yl)-4-propylbenzenesulfonamide.

The term “mammal” includes organisms, which include mice, rats, cows, sheep, pigs, rabbits, goats, and horses, monkeys, dogs, cats, and preferably humans.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/Kg to about 1000 mg/Kg, (e.g., from about 0.1 to about 100 mg/Kg, from about 1 to about 100 mg/Kg). Effective doses will also vary depending on route of administration, as well as the possibility of co-usage with other agents.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, Cl-C₂₀ alkyl indicates that the group may have from 1 to 20 (inclusive) carbon atoms in it. Any atom can be substituted. Examples of alkyl groups include without limitation methyl, ethyl, n-propyl, i-propyl and t-butyl.

The term “cycloalkyl” refers to saturated monocyclic, bicyclic, tricyclic, or other polycyclic hydrocarbon groups. Any atom can be substituted, e.g., by one or more substituents. Cycloalkyl groups can contain fused rings. Fused rings are rings that share a common carbon atom. Cycloalkyl moieties can include, e.g., cyclopropyl, cyclohexyl, methylcyclohexyl (the point of attachment to another moiety can be either the methyl group or a cyclohexyl ring carbon), adamantyl, and norbornyl.

The terms “haloalkyl” and “halocycloalkyl” refer to an alkyl or cycloalkyl group, respectively, in which at least one hydrogen atom is replaced by halo. In some embodiments, more than one hydrogen atom (2, 3, 4, 5, 6, 7, 8, 9, 10, 11, 12, 13, 14, 15, 16, 17, 18, 19, 20, 21, 22, 23, 24, 25, 26,etc. hydrogen atoms) on a alkyl or cycloalkyl group can be replaced by more than one halogens (e.g., 2, 3, 4, 5, 6, 7, 8, 9, 10, 11, 12, 13, 14, 15, 16, 17, 18, 19, 20, 21, 22, 23, 24, 25, 26, etc. hydrogen atoms), which can be the same or different. “Haloalkyl” and “halocycloalkyl” also include alkyl moieties in which all hydrogens have been replaced by halo (e.g., perhaloalkyl and perhalocycloalkyl, such as trifluoromethyl and perfluorocyclohexyl, respectively).

The term “aralkyl” refers to an alkyl moiety in which an alkyl hydrogen atom is replaced by an aryl group. Aralkyl includes groups in which more than one hydrogen atom on an alkyl moiety has been replaced by an aryl group. Any ring or chain atom can be substituted e.g., by one or more substituents. Examples of “aralkyl” include without limitation benzyl, 2-phenylethyl, 3-phenylpropyl, benzhydryl, and trityl groups.

The term “heteroaralkyl” refers to an alkyl moiety in which an alkyl hydrogen atom is replaced by a heteroaryl group. Heteroaralkyl includes groups in which more than one hydrogen atom on an alkyl moiety has been replaced by a heteroaryl group. Any ring or chain atom can be substituted e.g., by one or more substituents. Heteroaralkyl can include, for example, 2-pyridylethyl.

The term “alkenyl” refers to a straight or branched hydrocarbon chain containing 2-20 carbon atoms and having one or more double bonds. Any atom can be substituted, e.g., by one or more substituents. Alkenyl groups can include, e.g., allyl, propenyl, 2-butenyl, 3-hexenyl and 3-octenyl groups. One of the double bond carbons can optionally be the point of attachment of the alkenyl substituent. The term “alkynyl” refers to a straight or branched hydrocarbon chain containing 2-20 carbon atoms and having one or more triple bonds. Any atom can be substituted, e.g., by one or more substituents. Alkynyl groups can include, e.g., ethynyl, propargyl, and 3-hexynyl. One of the triple bond carbons can optionally be the point of attachment of the alkynyl substituent.

The term “alkoxy” refers to an —O-alkyl radical e.g. methoxy, ethoxy, etc. The term “mercapto” refers to an SH radical. The term “thioalkoxy” refers to an —S-alkyl radical, e.g. thiomethoxy, thioethoxy etc. The term aryloxy refers to an —O-aryl radical. The term thioaryloxy refers to an —S-aryl radical.

The term “heterocyclyl” refers to a monocyclic, bicyclic, tricyclic or other polycyclic ring system having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). The heteroatom can optionally be the point of attachment of the heterocyclyl substituent. Any atom can be substituted, e.g., by one or more substituents. The heterocyclyl groups can contain fused rings. Fused rings are rings that share a common carbon atom. Heterocyclyl groups can include, e.g., tetrahydrofuranyl, tetrahydropyranyl, piperidinyl, morpholino, pyrrolinyl, and pyrrolidinyl.

The term “cycloalkenyl” refers to partially unsaturated monocyclic, bicyclic, tricyclic, or other polycyclic hydrocarbon groups. The unsaturated carbon can optionally be the point of attachment of the cycloalkenyl substituent. Any atom can be substituted e.g., by one or more substituents. The cycloalkenyl groups can contain fused rings. Fused rings are rings that share a common carbon atom. Cycloalkenyl moieties can include, e.g., cyclohexenyl, cyclohexadienyl, norbornenyl, or cyclooctatetraenyl.

The term “heterocycloalkenyl” refers to partially unsaturated monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from 0, N, or S (e.g., carbon atoms and 1-4, 1-8, or 1-10 heteroatoms of N, O, or S if monocyclic, bicyclic, or tricyclic, respectively). The unsaturated carbon or the heteroatom can optionally be the point of attachment of the heterocycloalkenyl substituent. Any atom can be substituted, e.g., by one or more substituents. The heterocycloalkenyl groups can contain fused rings. Fused rings are rings that share a common carbon atom. Heterocycloalkenyl groups can include, e.g., tetrahydropyridyl, and dihydropyranyl.

The term “aryl” refers to an aromatic monocyclic, bicyclic, or tricyclic hydrocarbon ring system, wherein any ring atom can be substituted, e.g., by one or more substituents. Aryl groups can contain fused rings. Fused rings are rings that share a common carbon atom. Aryl moieties can include, e.g., phenyl, naphthyl, anthracenyl, and pyrenyl.

The term “heteroaryl” refers to an aromatic monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). Any atom can be substituted, e.g., by one or more substituents. Heteroaryl groups can contain fused rings. Fused rings are rings that share a common carbon atom. Heteroaryl groups include pyridyl, thienyl, furanyl, imidazolyl, pyrrolyl, isoxazolyl and benzthienyl.

The terms “arylcycloalkyl,” “arylcycloalkenyl,” “arylheterocyclyl,” and “arylheterocycloalkenyl” refer to bicyclic, tricyclic, or other polycyclic ring systems that include an aryl ring fused to a cycloalkyl, cycloalkenyl, heterocyclyl, and heterocycloalkenyl, respectively. Any atom can be substituted, e.g., by one or more substituents. For example, arylcycloalkyl can include fluorenyl and indanyl; arylcycloalkenyl can include indenyl; arylheterocyclyl can include 2,3-dihydrobenzofuranyl and 1,2,3,4-tetrahydroisoquinolinyl; and arylheterocycloalkenyl can include 1,4-dihydro-1,4-epoxynaphthalenyl.

The term “oxo” refers to an oxygen atom, which forms a carbonyl when attached to carbon, an N-oxide when attached to nitrogen, and a sulfoxide or sulfone when attached to sulfur.

The term “substituents” refers to a group “substituted” on, e.g., an alkyl, cycloalkyl, alkenyl, alkynyl, aralkyl, heteroaralkyl, heterocyclyl, heterocycloalkenyl, cycloalkenyl, aryl, or heteroaryl group at any atom of that group. In one aspect, the substituents on a group are independently any one single, or any subset of the aforementioned substituents. In another aspect, a substituent may itself be substituted with any one of the above substituents.

The details of one or more embodiments of the invention are set forth in the accompanying drawings and the description below. Other features and advantages of the invention will be apparent from the description and from the claims.

### DETAILED DESCRIPTION

This invention relates to sulfonamide 11-beta HSD1 inhibitor compounds, pharmaceutical compositions and related methods.

The sulfonamide 11-beta HSD1 inhibitor compounds have the general formula (I) below:

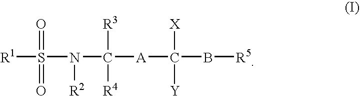

In some embodiments, R¹ and/or R⁵ can be C₁-C₂₀ (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkyl, optioanlly inserted with from 1-10 (e.g., 1, 2, 3, 4, 5, 6, 7, 8, 9, or 10) heteroatoms, which can be, independently of one another, nitrogen, oxygen or sulfur and/or optionally substituted with from 1-10 (e.g., 1, 2, 3, 4, 5, 6, 7, 8, 9, or 10) R^{a}.

Each R^{a} can be, independently of one another, NR^{g}R^{h}, nitro, hydroxy, oxo, thioxo, C₁-C₁₂ (e.g., C₁, C₂, C₃, C₄, C₅, C₆, C₇, C₈) C₉, C₁₀, C₁₁, or C₁₂) alkoxy, C₁-C₁₂ (e.g., C₁, C₂, C₃, C₄, C₅, C₆, C₇, C₈, C₉, C₁₀, C₁₁, or C₁₂) haloalkoxy, C₆-C₁₆ (e.g., C₆, C₇, C₈, C₉, C₁₀, C₁₁, C₁₂, C₁₃, C₁₄, C₁₅, or C₁₆) aryloxy, mercapto, C₁-C₁₂ (e.g., C₁, C₂, C₃, C₄, C₅, C₆, C₇, C₈, C₉, C₁₀, C₁₁, or C₁₂) thioalkoxy, C₆-C₁₆ (e.g., C₆, C₇, C₈, C₉, C₁₀, C₁₁, C₁₂, C₁₃, C₁₄, C₁₅, or C₁₆) thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h}). When two or more R^{a} substituents are present, R¹ can be substituted with any combination of the above set of substitutents.

Each R^{g}, R^{h}, and R^{j} can be, independently of one another, hydrogen; C₁-C₁₂ (e.g., C₁, C₂, C₃, C₄, C₅, C₆, C₇, C₈, C₉, C₁₀, C₁₁, or C₁₂) alkyl optionally inserted with from 1-6 (e.g., 1, 2, 3, 4, 5, or 6) heteroatoms, which can be, independently of one another, nitrogen, oxygen or sulfur; C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkenyl; C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkynyl; C₇-C₂₀ (e.g., C₇, C₈, C₉, C₁₀, C₁₁, C₁₂, C₁₃, C₁₄, C₁₅, C₁₆, C₁₇, C₁₈, C₁₉, or C₂₀) aralkyl; heteroaralkyl including 6-20 (e.g., 6, 7, 8, 9, 10, 11, 12, 13, 14, 15, 16, 17, 18, 19, or 20) atoms; C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkyl; C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkenyl; heterocyclyl including 3-16 (e.g., 3, 4, 5, 6, 7, 8, 9, 10, 11, 12, 13, 14, 15, or 16) atoms; heterocycloalkenyl including 3-16 (e.g., 3, 4, 5, 6, 7, 8, 9, 10, 11, 12, 13, 14, 15, or 16) atoms; C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkyl; C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkenyl; arylheterocyclyl including 8-20 (e.g., 8, 9, 10, 11, 12, 13, 14, 15, 16, 17, 18, 19, or 20) atoms; arylheterocycloalkenyl including 8-20 (e.g., 8, 9, 10, 11, 12, 13, 14, 15, 16, 17, 18, 19, or 20) atoms; C₆-C₁₆ (e.g., C₆, C₇, C₈, C₉, C₁₀, C₁₁, C₁₂, C₁₃, C₁₄, C₁₅, or C₁₆) aryl; or heteroaryl including 5-16 (e.g., 5, 6, 7, 8, 9, 10, 11, 12, 13, 14, 15, or 16) atoms.

Each R^{k} can be, independently of one another, hydrogen or C₁-C₁₀ (e.g., C₁, C₂, C₃, C₄, C₅, C₆, C₇, C₈, C₉, or C₁₀) alkyl.

Each R^{m} can be, independently of one another, hydrogen; C₁-C₁₂ (e.g., C₁, C₂, C₃, C₄, C₅, C₆, C₇, C₈, C₉, C₁₀, C₁₁, or C₁₂) alkyl optionally inserted with from 1-6 (e.g., 1, 2, 3, 4, 5, or 6) heteroatoms, which can be, independently of one another, nitrogen, oxygen or sulfur; C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkenyl; C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkynyl; C₇-C₂₀ (e.g., C₇, C₈, C₉, C₁₀, C₁₁, C₁₂, C₁₃, C₁₄, C₁₅, C₁₆, C₁₇, C₁₈, C₁₉, or C₂₀) aralkyl; heteroaralkyl including 6-20 (e.g., 6, 7, 8, 9, 10, 11, 12, 13, 14, 15, 16, 17, 18, 19, or 20) atoms; C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkyl; C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkenyl; heterocyclyl including 3-16 (e.g., 3, 4, 5, 6, 7, 8, 9, 10, 11, 12, 13, 14, 15, or 16) atoms; heterocycloalkenyl including 3-16 (e.g., 3, 4, 5, 6, 7, 8, 9, 10, 11, 12, 13, 14, 15, or 16) atoms; C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkyl; C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkenyl; arylheterocyclyl including 8-20 (e.g., 8, 9, 10, 11, 12, 13, 14, 15, 16, 17, 18, 19, or 20) atoms; arylheterocycloalkenyl including 8-20 (e.g., 8, 9, 10, 11, 12, 13, 14, 15, 16, 17, 18, 19, or 20) atoms; C₆-C₁₆ (e.g., C₆, C₇, C₈, C₉, C₁₀, C₁₁, C₁₂, C₁₃, C₁₄, C₁₅, or C₁₆) aryl; heteroaryl including 5-16 (e.g., 5, 6, 7, 8, 9, 10, 11, 12, 13, 14, 15, or 16) atoms; NR^{g}R^{h} or OR^{j}, in which R^{g}, R^{h}, and R^{j} can be as defined above.

Each R^{p} can be, independently of one another, C₁-C₁₂ (e.g., C₁, C₂, C₃, C₄, C₅, C₆, C₇, C₈, C₉, C₁₀, C₁₁, or C₁₂) alkyl optionally inserted with from 1-6 (e.g., 1, 2, 3, 4, 5, or 6) heteroatoms, which can be, independently of one another, nitrogen, oxygen or sulfur; C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkenyl; C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkynyl; C₇-C₂₀ (e.g., C₇, C₈, C₉, C₁₀, C₁₁, C₁₂, C₁₃, C₁₄, C₁₅, C₁₆, C₁₇, C₁₈, C₁₉, or C₂₀) aralkyl; heteroaralkyl including 6-20 (e.g., 6, 7, 8, 9, 10, 11, 12, 13, 14, 15, 16, 17, 18, 19, or 20) atoms; C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkyl; C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkenyl; heterocyclyl including 3-16 (e.g., 3, 4, 5, 6, 7, 8, 9, 10, 11, 12, 13, 14, 15, or 16) atoms; heterocycloalkenyl including 3-16 (e.g., 3, 4, 5, 6, 7, 8, 9, 10, 11, 12, 13, 14, 15, or 16) atoms; C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkyl; C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkenyl; arylheterocyclyl including 8-20 (e.g., 8, 9, 10, 11, 12, 13, 14, 15, 16, 17, 18, 19, or 20) atoms; arylheterocycloalkenyl including 8-20 (e.g., 8, 9, 10, 11, 12, 13, 14, 15, 16, 17, 18, 19, or 20) atoms; C₆-C₁₆ (e.g., C₆, C₇, C₈, C₉, C₁₀, C₁₁, C₁₂, C₁₃, C₁₄, C₁₅, or C₁₆) aryl; heteroaryl including 5-16 (e.g., 5, 6, 7, 8, 9, 10, 11, 12, 13, 14, 15, or 16) atoms; NR^{g}R^{h}, or OR^{j}, in which n can be 1 or 2, and R^{g}, R^{h}, and R^{j} can be as defined above.

In some embodiments, R¹ and/or R⁵ can be C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkyl, optionally substituted with from 1-10 (e.g., 1, 2, 3, 4, 5, 6, 7, 8, 9, or 10) R^{a}. Each R^{a} can be, independently of one another, as defined above.

In some embodiments, R¹ and/or R⁵ can be C₁-C₂₀ (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) haloalkyl or C₃-C₁₆ (e.g., C₃, C₄, C₅, C₆, C₇, C₈, C₉, C₁₀, C₁₁, C₁₂, C₁₃, C₁₄, C₁₅, or C₁₆) halocycloalkyl, each of which can be optionally substituted with from 1-10 (e.g., 1, 2, 3, 4, 5, 6, 7, 8, 9, or 10) R^{a}. Each R^{a} can be, independently of one another, as defined above.

In some embodiments, R¹ and/or R⁵ can be C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkenyl, C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkynyl, C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkenyl, heterocyclyl including 3-16 (e.g., 3, 4, 5, 6, 7, 8, 9, 10, 11, 12, 13, 14, 15, or 16) atoms, or heterocycloalkenyl including 3-16 (e.g., 3, 4, 5, 6, 7, 8, 9, 10, 11, 12, 13, 14, 15, or 16) atoms, each of which can be optionally substituted with from 1-10 (e.g., 1, 2, 3, 4, 5, 6, 7, 8, 9, or 10) R^{b}.

Each R^{b} can be, independently of one another, halo, NR^{g}R^{h}, nitro, hydroxy, oxo, thioxo, C₁-C₁₂ (e.g., C₁, C₂, C₃, C₄, C₅, C₆, C₇, C₈, C₉, C₁₀, C₁₁, or C₁₂) alkoxy, C₁-C₁₂ (e.g., C₁, C₂, C₃, C₄, C₅, C₆, C₇, C₈, C₉, C₁₀, C₁₁, or C₁₂) haloalkoxy, C₆-C₁₆ (e.g., C₆, C₇, C₈, C₉, C₁₀, C₁₁, C₁₂, C₁₃, C₁₄, C₁₅, or C₁₆) aryloxy, mercapto, C₁-C₁₂ (e.g., C₁, C₂, C₃, C₄, C₅, C₆, C₇, C₈, C₉, C₁₀, C₁₁, or C₁₂) thioalkoxy, C₆-C₁₆ (e.g., C₆, C₇, C₈, C₉, C₁₀, C₁₁, C₁₂, C₁₃, C₁₄, C₁₅, or C₁₆) thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h}). When two or more R^{b} substituents are present, R¹ can be substituted with any combination of the above set of substitutents. Each R^{g}, R^{h}, R^{j}, R^{m}, n, and R^{p} can be, independently of one another, as defined above.

In some embodiments, R¹ and/or R⁵ can be C₇-C₂₀ (e.g., C₇, C₈, C₉, C₁₀, C₁₁, C₁₂, C₁₃, C₁₄, C₁₅, C₁₆, C₁₇, C₁₈, C₁₉, or C₂₀) aralkyl, heteroaralkyl including 6-20 (e.g., 6, 7, 8, 9, 10, 11, 12, 13, 14, 15, or 16) atoms, C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkyl; C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkenyl; arylheterocyclyl including 8-20 (e.g., 8, 9, 10, 11, 12, 13, 14, 15, 16, 17, 18, 19, or 20) atoms; or arylheterocycloalkenyl including 8-20 (e.g., 8, 9, 10, 11, 12, 13, 14, 15, 16, 17, 18, 19, or 20) atoms;each of which can be optionally substituted with from 1-10 (e.g., 1, 2, 3, 4, 5, 6, 7, 8, 9, or 10) R^{c}.

Each R^{c} can be, independently of one another, C₁-C₁₂ (e.g., C₁, C₂, C₃, C₄, C₅, C₆, C₇, C₈, C₉, C₁₀, C₁₁, or C₁₂) haloalkyl, halo, NR^{g}R^{h}, nitro, hydroxy, oxo, thioxo, C₁-C₁₂ (e.g., C₁, C₂, C₃, C₄, C₅, C₆, C₇, C₈, C₉, C₁₀, C₁₁, or C₁₂) alkoxy, C₁-C₁₂ (e.g., C₁, C₂, C₃, C₄, C₅, C₆, C₇, C₈, C₉, C₁₀, C₁₁, or C₁₂) haloalkoxy, C₆-C₁₆ (e.g., C₆, C₇, C₈, C₉, C₁₀, C₁₁, C₁₂, C₁₃, C₁₄, C₁₅, or C₁₆) aryloxy, mercapto, C₁-C₁₂ (e.g., C₁, C₂, C₃, C₄, C₅, C₆, C₇, C₈, C₉, C₁₀, C₁₁, or C₁₂) thioalkoxy, C₆-C₁₆ (e.g., C₆, C₇, C₈, C₉, C₁₀, C₁₁, C₁₂, C₁₃, C₁₄, C₁₅, or C₁₆) thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h}). When two or more R^{c} substituents are present, R¹ can be substituted with any combination of the above set of substitutents. Each R^{g}, R^{h}, R^{j}, R^{m}, n, and R^{p} can be, independently of one another, as defined above.

In some embodiments, R¹ and/or R⁵ can be C₆-C₁₆ (e.g., C₆, C₇, C₈, C₉, C₁₀, C₁₁, C₁₂, C₁₃, C₁₄, C₁₅, or C₁₆) aryl or heteroaryl including 5-16 (e.g., 5, 6, 7, 8, 9, 10, 11, 12, 13, 14, 15, or 16) atoms, each of which can be optionally substituted with from 1-10 (e.g., 1, 2, 3, 4, 5, 6, 7, 8, 9, or 10) R^{d}.

Each R^{d} can be, independently of one another:

(i) halo, NR^{g}R^{h}, nitro, hydroxy, oxo, thioxo, C₁-C₁₂ (e.g., C₁, C₂, C₃, C₄, C₅, C₆, C₇, C₈, C₉, C₁₀, C₁₁, or C₁₂) alkoxy, C₁-C₁₂ (e.g., C₁, C₂, C₃, C₄, C₅, C₆, C₇, C₈, C₉, C₁₀, C₁₁, or C₁₂) haloalkoxy, C₆-C₁₆ (e.g., C₆, C₇, C₈, C₉, C₁₀, C₁₁, C₁₂, C₁₃, C₁₄, C₁₅, or C₁₆) aryloxy, mercapto, C₁-C₁₂ (e.g., C₁, C₂, C₃, C₄, C₅, C₆, C₇, C₈, C₉, C₁₀, C₁₁, or C₁₂) thioalkoxy, C₆-C₁₆ (e.g., C₆, C₇, C₈, C₉, C₁₀, C₁₁, C₁₂, C₁₃, C₁₄, C₁₅, or C₁₆) thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h}); or

(ii) C₁-C₁₂ (e.g., C₁, C₂, C₃, C₄, C₅, C₆, C₇, C₈, C₉, C₁₀, C₁₁, or C₁₂) alkyl, optionally inserted with from 1-6 (e.g., 1, 2, 3, 4, 5, or 6) heteroatoms, which can be, independently of one another, nitrogen, oxygen or sulfur and/or optionally substituted with from 1-10 (e.g., 1, 2, 3, 4, 5, 6, 7, 8, 9, or 10) R^{a}; or

(iii) C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkenyl, C₂-C₂₀ (e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkynyl, C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkenyl, heterocyclyl including 3-16 (e.g., 3, 4, 5, 6, 7, 8, 9, 10, 11, 12, 13, 14, 15, or 16) atoms, or heterocycloalkenyl including 3-16 (e.g., 3, 4, 5, 6, 7, 8, 9, 10, 11, 12, 13, 14, 15, or 16) atoms, each of which can be optionally substituted with from 1-10 (e.g., 1, 2, 3, 4, 5, 6, 7, 8, 9, or 10) R^{b}; or

(iv) C₁-C₁₂ (e.g., C₁, C₂, C₃, C₄, C₅, C₆, C₇, C₈, C₉, C₁₀, C₁₁, or C₁₂) haloalkyl; or

(v) C₇-C₂₀ (e.g., C₇, C₈, C₉, C₁₀, C₁₁, C₁₂, C₁₃, C₁₄, C₁₅, C₁₆, C₁₇, C₁₈, C₁₉, or C₂₀) aralkyl or heteroaralkyl including 6-20 (e.g., 6, 7, 8, 9, 10, 11, 12, 13, 14, 15, or 16) atoms, each of which can be optionally substituted with from 1-10 (e.g., 1, 2, 3, 4, 5, 6, 7, 8, 9, or 10) R^{c}; or

(vi) C₆-C₁₆ (e.g., C₆, C₇, C₈, C₉, C₁₀, C₁₁, C₁₂, C₁₃, C₁₄, C₁₅, or C₁₆) aryl or heteroaryl including 5-16 (e.g., 5, 6, 7, 8, 9, 10, 11, 12, 13, 14, 15, or 16) atoms, each of which can be optionally substituted with C₁-C₁₂ (e.g., C₁, C₂, C₃, C₄, C₅, C₆, C₇, C₈, C₉, C₁₀, C₁₁, or C₁₂) alkyl, C₁-C₁₂ (e.g., C₁, C₂, C₃, C₄, C₅, C₆, C₇, C₈, C₉, C₁₀, C₁₁, or C₁₂) haloalkyl, halo, NR^{g}R^{h}, nitro, hydroxy, oxo, thioxo, C₁-C₁₂ (e.g., C₁, C₂, C₃, C₄, C₅, C₆, C₇, C₈, C₉, C₁₀, C₁₁, or C₁₂) alkoxy, C₁-C₁₂ (e.g., C₁, C₂, C₃, C₄, C₅, C₆, C₇, C₈, C₉, C₁₀, C₁₁, or C₁₂) haloalkoxy, C₆-C₁₆ (e.g., C₆, C₇, C₈, C₉, C₁₀, C₁₁, C₁₂, C₁₃, C₁₄, C₁₅, or C₁₆) aryloxy, mercapto, C₁-C₁₂ (e.g., C₁, C₂, C₃, C₄, C₅, C₆, C₇, C₈, C₉, C₁₀, C₁₁, or C₁₂) thioalkoxy, C₆-C₁₆ (e.g., C₆, C₇, C₈, C₉, C₁₀, C₁₁, C₁₂, C₁₃, C₁₄, C₁₅, or C₁₆) thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h}).

When two or more R^{d} substituents are present, R¹ can be substituted with any combination of the above set of substitutents. Each R^{a}, R^{b}, R^{c}, R^{g}, R^{h}, R^{j}, R^{m}, n, and R^{p} can be, independently of one another, as defined above.

In some embodiments, R¹ together with R³ or R⁴ can be heterocyclyl including 3-10 (e.g., 3, 4, 5, 6, 7, 8, 9, or 10) atoms or heterocycloalkenyl including 5-10 (e.g., 3, 4, 5, 6, 7, 8, 9, or 10) atoms, each of which can be optionally substituted with from 1-5 (e.g., 1, 2, 3, 4, or 5) R^{b}; or arylheterocyclyl including 8-12 (e.g., 8, 9, 10, 11, or 12) atoms or arylheterocycloalkenyl including 8-12 (e.g., 8, 9, 10, 11, or 12) atoms, each of which can be optionally substituted with from 1-5 (e.g., 1, 2, 3, 4, or 5) R^{c}. Each R^{b} and R^{c} can be, independently of one another, as defined above.

In some embodiments, R⁵ together with R³ or R⁴ can be C₃-C₁₀ (e.g., C₃, C₄, C₅, C₆, C₇, C₈, C₉, or C₁₀) cycloalkyl, optionally substituted with from 1-5 (e.g., 1, 2, 3, 4, or 5) R^{a}; C₃-C₁₀ (e.g., C₃, C₄, C₅, C₆, C₇, C₈, C₉, or C₁₀) halocycloalkyl; C₃-C₁₀ (e.g., C₃, C₄, C₅, C₆, C₇, C₈, C₉, or C₁₀) cycloalkenyl, heterocyclyl including 5-10 (e.g., 5, 6, 7, 8, 9, or 10) atoms, or heterocycloalkenyl including 5-10 (e.g., 5, 6, 7, 8, 9, or 10) atoms, each of which is optionally substituted with from 1-5 (e.g., 1, 2, 3, 4, or 5) R^{b}; or C₈-C₁₂ (e.g., C₈, C₉, C₁₀, C₁₁, or C₁₂) arylcycloalkyl, C₈-C₁₂ (e.g., C₈, C₉, C₁₀, C₁₁, or C₁₂) arylcycloalkenyl, arylheterocyclyl including 8-12 (e.g., 8, 9, 10, 11, or 12) atoms, or arylheterocycloalkenyl including 8-12 (e.g., 8, 9, 10, 11, or 12) atoms,each of which can be optionally substituted with from 1-5 (e.g., 1, 2, 3, 4, or 5) R^{c}. Each R^{a}, R^{b}, and R^{c} can be, independently of one another, as defined above.

In some embodiments, R¹ is not C₁-C₁₀ alkyl, optionally substituted with from 1-10 R^{a} and/or optionally inserted with nitrogen, oxygen or sulfur; C₁-C₁₀ haloalkyl; C₃-C₁₆ cycloalkyl, optionally substituted with from 1-10 R^{a}; C₂-Clo alkenyl; C₂-C₁₀ alkynyl; C₃-C₁₆ cycloalkenyl; heterocyclyl including 3-6 atoms, or heterocycloalkenyl including 3-6 atoms, C₇-C₂₀ aralkyl or heteroaralkyl including 6-20 atoms, each of which is optionally substituted with from 1-10 R^{c}; C₆ aryl, optionally substituted with 1-2 R^{d}; or heteroaryl including 5 atoms, optionally substituted with 1-2 R^{d}.

In some embodiments, R² can be hydrogen; C₁-C₂₀ (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀) alkyl or C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkyl, each of which is optionally substituted with from 1-10 (e.g., 1, 2, 3, 4, 5, 6, 7, 8, 9, or 10) R^{a}; C₆-C₁₆ (e.g., C₆, C₇, C₈, C₉, C₁₀, C₁₁, C₁₂, C₁₃, C₁₄, C₁₅, or C₁₆) aryl, optionally substituted with from 1-10 (e.g., 1, 2, 3, 4, 5, 6, 7, 8, 9, or 10) R^{d}; or C₇-C₂₀ (e.g., C₇, C₈, C₉, C₁₀, C₁₁, C₁₂, C₁₃, C₁₄, C₁₅, C₁₆, C₁₇, C₁₈, C₁₉, or C₂₀) aralkyl or heteroaralkyl including 6-20 (e.g., 6, 7, 8, 9, 10, 11, 12, 13, 14, 15, 16, 17, 18, 19, or 20) atoms, each of which is optionally substituted with 1-10 (e.g., 1, 2, 3, 4, 5, 6, 7, 8, 9, or 10) R^{c}. Each R^{a}, R^{c}, and R^{d} can be, independently of one another, as defined above.

In some embodiments, R³ and R⁴ can be, independently of one another, hydrogen or C₁-C₁₀ (e.g., C₁, C₂, C₃, C₄, C₅, C₆, C₇, C₈, C₉, or C₁₀) alkyl. In certain embodiments, R³ and R⁴ can be the same (e.g., both are hydrogen or both are C₁-C₁₀ alkyl, e.g., CH₃) or different (e.g., one of R³ and R⁴ can be hydrogen and the other can be C₁-C₁₀ alkyl, e.g., CH₃ or isopropyl; or R³ and R⁴ can both be C₁-C₁₀ alkyl with each of R³ and R⁴ having a different carbon content, e.g., R³ can be C₂ alkyl and R⁴ can be C₃ alkyl).

In some embodiments, R³ and R⁴ together can be C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkyl, optionally substituted with from 1-10 (e.g., 1, 2, 3, 4, 5, 6, 7, 8, 9, or 10) R^{a}; C₃-C₁₆ (e.g., C₃, C₄, C₅, C₆, C₇, C₈, C₉, C₁₀, C₁₁, C₁₂, C₁₃, C₁₄, C₁₅, or C₁₆) halocycloalkyl; C₃-C₁₆ (e.g., C₃, C₄, C₅, C₆, C₇, C₈, C₉, C₁₀, C₁₁, C₁₂, C₁₃, C₁₄, C₁₅, or C₁₆) cycloalkenyl, heterocyclyl including 5-16 (e.g., 5, 6, 7, 8, 9, 10, 11, 12, 13, 14, 15, or 16) atoms, or heterocycloalkenyl including 5-16 (e.g., 5, 6, 7, 8, 9, 10, 11, 12, 13, 14, 15, or 16) atoms, each of which can be optionally substituted with from 1-10 (e.g., 1, 2, 3, 4, 5, 6, 7, 8, 9, or 10) R^{b}; or C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkyl; C₈-C₂₀ (e.g., C₈, C₉, C₁₀, C₁₁, C₁₂, C₁₃, C₁₄, C₁₅, C₁₆, C₁₇, C₁₈, C₁₉, or C₂₀) arylcycloalkenyl; arylheterocyclyl including 8-20 (e.g., 8, 9, 10, 11, 12, 13, 14, 15, 16, 17, 18, 19, or 20) atoms; or arylheterocycloalkenyl including 8-20 (e.g., 8, 9, 10, 11, 12, 13, 14, 15, 16, 17, 18, 19, or 20) atoms; each of which can be optionally substituted with from 1-10 (e.g., 1, 2, 3, 4, 5, 6, 7, 8, 9, or 10) R^{c}. Each R^{a}, R^{b}, and R^{c} can be, independently of one another, as defined above.

In some embodiments, one of R³ or R⁴ can be hydrogen or C₁-C₁₀ (e.g., C₁, C₂, C₃, C₄, C₅, C₆, C₇, C₈, C₉, or C₁₀) alkyl, and the other together with R¹ can be heterocyclyl including 3-10 (e.g., 3, 4, 5, 6, 7, 8, 9, or 10) atoms or heterocycloalkenyl including 5-10 (e.g., 3, 4, 5, 6, 7, 8, 9, or 10) atoms, each of which can be optionally substituted with from 1-5 (e.g., 1, 2, 3, 4, or 5) R^{b}; or arylheterocyclyl including 8-12 (e.g., 8, 9, 10, 11, or 12) atoms or arylheterocycloalkenyl including 8-12 (e.g., 8, 9, 10, 11, or 12) atoms, each of which can be optionally substituted with from 1-5 (e.g., 1, 2, 3, 4, or 5) R^{c}. Each R^{b} and R^{c} can be, independently of one another, as defined above. Each R^{b} and R^{c} can be, independently of one another, as defined above.

In some embodiments, one of R³ or R⁴ can be hydrogen or C₁-C₁₀ (e.g., C₁, C₂, C₃, C₄, C₅, C₆, C₇, C₈, C₉, or C₁₀) alkyl, and the other together with R⁵ can be C₃-C₁₀ (e.g., C₃, C₄, C₅, C₆, C₇, C₈, C₉, or C₁₀) cycloalkyl, optionally substituted with from 1-5 (e.g., 1, 2, 3, 4, or 5) R^{a}; C₃-C₁₀ (e.g., C₃, C₄, C₅, C₆, C₇, C₈, C₉, or C₁₀) halocycloalkyl; C₃-C₁₀ (e.g., C₃, C₄, C₅, C₆, C₇, C₈, C₉, or C₁₀) cycloalkenyl, heterocyclyl including 5-10 (e.g., 5, 6, 7, 8, 9, or 10) atoms, or heterocycloalkenyl including 5-10 (e.g., 5, 6, 7, 8, 9, or 10) atoms, each of which is optionally substituted with from 1-5 (e.g., 1, 2,3, 4, or 5) R^{b}; or C₈-C₁₂ (e.g., C₈, C₉, C₁₀, C₁₁, or C₁₂) arylcycloalkyl, C₈-C₁₂ (e.g., C₈, C₉, C₁₀, C₁₁, or C₁₂) arylcycloalkenyl, arylheterocyclyl including 8-12 (e.g., 8, 9, 10, 11, or 12) atoms, or arylheterocycloalkenyl including 8-12 (e.g., 8, 9, 10, 11, or 12) atoms,each of which can be optionally substituted with from 1-5 (e.g., 1, 2, 3, 4, or 5) R^{c}. Each R^{a}, R^{b}, and R^{c} can be, independently of one another, as defined above. Each R^{a} R^{b}, and R^{c} can be, independently of one another, as defined above.

In some embodiments, A and B can be, independently of one another, a bond or (CR^{e}R^{f})ₘ, in which m can be 1-20 (e.g., 1, 2, 3, 4, 5, 6, 7, 8, 9, 10, 11, 12, 13, 14, 15, 16, 17, 18, 19, or 20), and R^{e} and R^{f} can be, independently of one another, hydrogen or C₁-C₁₀ (e.g., C₁, C₂, C₃, C₄, C₅, C₆, C₇, C₈, C₉, or C₁₀) alkyl. In certain embodiments, A and B can be the same (e.g., both can be a bond or both can be (CR^{e}R^{f})ₘ, e.g., —CH₂—) or different (e.g., one of A and B can be a bond and the other can be (CR^{e}R^{f})m, e.g., —CH₂—; or A and B can both be (CR^{e}R^{f})ₘ with each of A and B having a different value of m, e.g., A can m=2 and B can have m=3). In certain embodiments, R^{e} and R^{f} can be the same (e.g., both are hydrogen or both can be C₁-C₁₀ alkyl, e.g., CH₃) or different (e.g., one of R^{e} and R^{f} can be hydrogen and the other can be C₁-C₁₀ alkyl, e.g., CH₃; or R^{e} and R^{f} can both be C₁-C₁₀ alkyl with each of R^{e} and R^{f} having a different carbon content, e.g., R^{e} can be C₂ alkyl and R^{f} can be C₃ alkyl).

In some embodiments, X and Y together can be oxo. In other embodiments, X and Y can be, independently of one another, hydrogen, C₁-C₆ (e.g., C₁, C₂, C₃, C₄, C₅, or C₆) alkyl, or hydroxy. In certain embodiments, X and Y can be the same (e.g., both are hydrogen or both can be C₁-C₁₀ alkyl, e.g., CH₃) or different (e.g., one of X and Y can be hydrogen and the other can be C₁-C₁₀ alkyl or hydroxy; or X and Y can both be C₁-C₁₀ alkyl with each of X and Y having a different carbon content, e.g., X can be C₂ alkyl and Y can be C₃ alkyl).

In all embodiments, it is provided that when R¹ is isopropyl and X and Y together are oxo (e.g., when R¹ is isopropyl, X and Y together are oxo, and A and B are both a bond; e.g., when R¹ is isopropyl, X and Y together are oxo, A and B are both a bond, and R³ and R⁴ are both hydrogen), then R⁵ is not 4-bromophenyl, 4-benzamidophenyl, 4-methyl-phenyl, 4-isopropylphenyl, 4-isobutylphenyl, 4-t-butylphenyl, 4-methoxyphenyl, 4-isopropoxyphenyl, 4-cyclopentylphenyl, 4-cyclohexylphenyl, 4-(2-hydroxy-methylphenyl)phenyl, 4-(4-hydroxymethylphenyl)phenyl, 4-(2-furyl)phenyl, 4-(3-furyl)phenyl, 4-(2-thienyl)phenyl, 4-(3-thienyl)phenyl, 4-(pyrrolidin-1-yl)phenyl, 4-(piperidin-1-yl)phenyl, 3-chloro-4-piperidin-1-ylphenyl, 4-(2-fluorophenyl)phenyl, 4-(3-fluorophenyl)phenyl, 4-(2-formylphenyl)phenyl, 4-(3-formylphenyl)phenyl, 4-(4-formylphenyl)phenyl, 4-(4-methylphenyl)phenyl, 4-(4-hydroxphenyl)phenyl, 4-(2-methoxyphenyl)phenyl or 4-(4-methoxyphenyl)phenyl.

For ease of exposition, it is understood that any recitation of ranges (e.g., C₁-C₂₀) or subranges of a particular range (e.g., C₁-C₄, C₂-C₆) for any of R¹, R², R³, R⁴, R⁵, A, B, X, Y, n, m, R^{a}, R^{b}, R^{c}, R^{d}, R^{e}, R^{f}, R^{g}, R^{h}, R^{j}, R^{k}, R^{m}, or R^{p} expressly includes each of the individual values that fall within the recited range, including the upper and lower limits of the recited range. For example, the range C₁-C₄ alkyl is understood to mean (e.g., C₁, C₂, C₃, or C₄) alkyl.

In some embodiments, when R^{c} is attached to an aryl or heteroaryl moiety, R^{c} can further include as permissible substituents: C₁-C₁₂ alkyl optionally substituted with from 1-10 R^{a} and/or optionally inserted with from 1-6 heteroatoms selected from the group consisting of nitrogen, oxygen or sulfur; or C₁-C₁₂ haloalkyl substituted with from 1-10 R^{a}; or C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which can be optionally substituted with from 1-10 R^{b}; or C₇-C₂₀ aralkyl or heteroaralkyl including 6-20 atoms, each of which can be optionally substituted with from 1-10 R^{c}.

In some embodiments, when R^{d} is a substituted C₆-C₁₆ aryl or heteroaryl including 5-16 atoms, then the permissible substitutents for the substituted C₆-C₁₆ aryl or heteroaryl including 5-16 atoms can further include: C₁-C₁₂ alkyl substituted with from 1-10 R^{a} and/or optionally inserted with from 1-6 heteroatoms selected from the group consisting of nitrogen, oxygen or sulfur; or C₁-C₁₂ haloalkyl substituted with from 1-10 R^{a}; or C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which can be optionally substituted with from 1-10 R^{b}; or C₇-C₂₀ aralkyl or heteroaralkyl including 6-20 atoms, each of which can be optionally substituted with from 1-10 R^{c}.

A subset of compounds include those having formula (II) in which R³ and R⁴ can be, independently of one another, hydrogen or C₁-C₁₀ alkyl, each of A and B are a bond, and X and Y together are oxo:

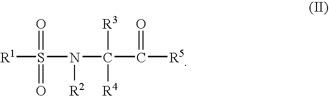

R¹, R², R³, R⁴, and R⁵ can be as defined throughout herein.

In some embodiments, R¹ can be selected from C₆-C₁₆ aryl, optionally substituted with from 1-10 R^{d}; heteroaryl including 5-16 atoms, optionally substituted with from 1-10 R^{d}; C₇-C₂₀ aralkyl, optionally substituted with from 1-10 R^{c}; or C₁-C₂₀ alkyl.

In some embodiments, R¹ can be C₆-C₁₀ aryl, optionally substituted with from 1-3 R^{d}. In certain embodiments, R¹ can be naphthyl (e.g., 1-naphthyl, 2-naphthyl). In other embodiments, R¹ can be phenyl, optionally substituted with from 1-3 R^{d}. Each R^{d} can be, independently of one another, C₁-C₆ alkyl (e.g., methyl, n-propyl, tert-butyl) C₆-C₁₀ aryl(e.g., phenyl), C₁-C₆ alkoxy (e.g., methoxy), halo (e.g. F, Cl, Br), C₆-C₁₀ aryloxy (e.g., phenoxy), cyano or nitro. In some embodiments, R¹ can be an ortho (o), meta (m), or para (p), monosubstituted phenyl (i.e., 1 R^{d}). In other embodiments, R¹ can be an o,o; o,m; m,m or m,p disubstituted phenyl (i.e., 2 R^{d}). Exemplary R¹ groups can include without limitation phenyl, 4-tert-butylphenyl, 4-biphenyl, 4-chlorophenyl, 3,5-dimethylphenyl, 4-bromophenyl, or 2-fluorophenyl.

In some embodiments, R¹ can be heteroaryl including 5-10 atoms, optionally substituted with from 1-2 R^{d}. In certain embodiments, R¹ can be thienyl, furyl, imidazolyl, or isoxazolyl, each optionally substituted with from 1-2 R^{d}. Each R^{d} can be, independently of one another, C₁-C₆ alkyl (e.g., CH₃), C₁-C₆ haloalkyl (e.g., CF₃), or halo (e.g., Cl).

In some embodiments, R¹ can be C₇-C₁₀ aralkyl, optionally substituted with from 1-2 R^{c}. In certain embodiments, R¹ can be benzyl or 2-phenylethyl, optionally substituted with halo (e.g., Cl, e.g., 4′-chlorobenzyl).

In some embodiments, R¹ can be C₁-C₁₂ alkyl (e.g., methyl, ethyl, propyl, or isopropyl). In certain embodiments, R¹ can be methyl, ethyl, or propyl. In other embodiments, R¹ can be isopropyl.

In some embodiments, R⁵ can be C₆-C₁₀ aryl, optionally substituted with from 1-2 R^{d}. In certain embodiments, R¹ can be naphthyl (e.g., 1-naphthyl, 2-naphthyl). In other embodiments, R⁵ can be phenyl optionally substituted with from 1-2 R^{d}. Each R^{d} can be, independently of one another, C₁-C₆ haloalkyl (e.g., CF₃) C₆-C₁₀ aryl (e.g., phenyl), or C₁-C₆ alkoxy (e.g., methoxy). Exemplary R¹ groups can include without limitation phenyl, 4-biphenyl, 4-trifluoromethyl, and 4-methoxyphenyl. In certain embodiments, R¹ groups can be phenyl, 4-biphenyl, 4-trifluoromethyl. In other embodiments, R¹ groups can be 4-methoxyphenyl.

In some embodiments, R⁵ can be heteroaryl including 5-10 atoms, optionally substituted with from 1-2 R^{d}. In certain embodiments, R⁵ can be thienyl, benzothienyl, furyl, imidazolyl, or isoxazolyl each, optionally substituted with from 1-2 R^{d}. Each R^{d} can be, independently of one another, C₁-C₆ alkyl (e.g., CH₃), C₁-C₆ haloalkyl (e.g., CF₃), or halo (e.g., Cl).

In some embodiments, R² can be hydrogen.

In some embodiments, R² can be C₁-C₂₀ alkyl (e.g., methyl or ethyl) or C₃-C₁₆ cycloalkyl, each of which is optionally substituted with from 1-10 R^{a}; C₆-C₁₆ aryl, optionally substituted with from 1-10 R^{d}; or C₇-C₂₀ aralkyl or heteroaralkyl including 6-20 atoms, each of which is optionally substituted with 1-5 R^{c}. In certain embodiments, R² is C₇-C₁₀ aralkyl, optionally substituted with oxo or C₁-C₄ haloalkyl (e.g., PhC(O)CH₂— or CF₃, e.g., 4′-trifluoromethylbenzyl).

In some embodiments, each of R³ and R⁴ can be hydrogen.

In some embodiments, each of R³ and R⁴ can be C₁-C₆ alkyl (e.g., methyl).

In some embodiments, one of R³ or R⁴ is hydrogen, and the other is C₁-C₆ alkyl (e.g., methyl or isopropyl).

Exemplary compounds of Formula (II) are delineated in the Examples.

In some embodiments, R¹ together with one of R³ or R⁴ can be a cyclic moiety, e.g., heterocyclyl, heterocycloalkenyl, arylheterocyclyl, or arylheterocycloalkenyl.

In some embodiments, one of R³ or R⁴ can be hydrogen or C₁-C₁₀ alkyl, and the other together with R¹ can be heterocyclyl including 3-10 atoms or heterocycloalkenyl including 5-10 atoms, each of which can be optionally substituted with from 1-5 R^{b}; or arylheterocyclyl including 8-12 atoms or arylheterocycloalkenyl including 8-12 atoms, each of which can be optionally substituted with from 1-5 R^{c}.

In certain embodiments, one of R³ or R⁴ is hydrogen, and the other together with R¹ is arylheterocyclyl including 9-12 atoms. A subset of compounds includes those in which A and B is a bond, and X and Y together are oxo. Exemplary compounds of this subset can have formula (III), in which q can be 0, 1, 2, or 3. In certain embodiments, R^{r} and R^{s} can be the same (e.g., both are hydrogen or both can be C₁-C₁₀ alkyl, e.g., CH₃) or different (e.g., one of R^{r} and R^{s} can be hydrogen and the other can be C₁-C₁₀ alkyl, e.g., CH₃; or R^{r} and R^{s} can both be C₁-C₁₀ alkyl with each of R^{r} and R^{s} having a different carbon content, e.g., R^{r} can be C₂ alkyl and R^{s} can be C₃ alkyl). R² and R⁵ can be as described elsewhere.

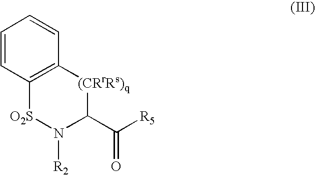

In some embodiments, R⁵ together with one of R³ or R⁴ can be a cyclic moiety, e.g., cycloalkyl, halocycloalkyl, cycloalkenyl, heterocyclyl, heterocycloalkenyl, arylcycloalkyl, arylcycloalkenyl, arylheterocyclyl, or arylheterocycloalkenyl.

In some embodiments, one of R³ or R⁴ can be hydrogen or C₁-C₁₀ alkyl, and the other together with R⁵ can be C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which can be optionally substituted with from 1-5 R^{c}.

In certain embodiments, one of R³ or R⁴ is hydrogen, and the other together with R⁵ is C₈-C₁₂ arylcycloalkyl. A subset of compounds includes those in which each of A and B is a bond, and X and Y together are oxo. Exemplary compounds of this subset can have formula (IV), in which z can be, 1, 2, 3, 4, or 5. In certain embodiments, R^{r} and R^{s} can be the same (e.g., both are hydrogen or both can be C₁-C₁₀ alkyl, e.g., CH₃) or different (e.g., one of R^{r} and R^{s} can be hydrogen and the other can be C₁-C₁₀ alkyl, e.g., CH₃; or R^{r} and R^{s} can both be C₁-C₁₀ alkyl with each of R^{r} and R^{s} having a different carbon content, e.g., R^{r} can be C₂ alkyl and R^{s} can be C₃ alkyl). R¹ and R² can be as described elsewhere.

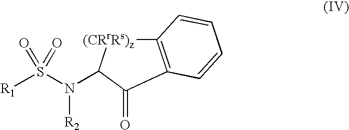

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species.

The compounds described herein can be synthesized according to methods described herein and/or conventional, organic chemical synthesis methods from commercially available starting materials and reagents. The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In some embodiments, the compounds described herein can be prepared according to the general schemes below:

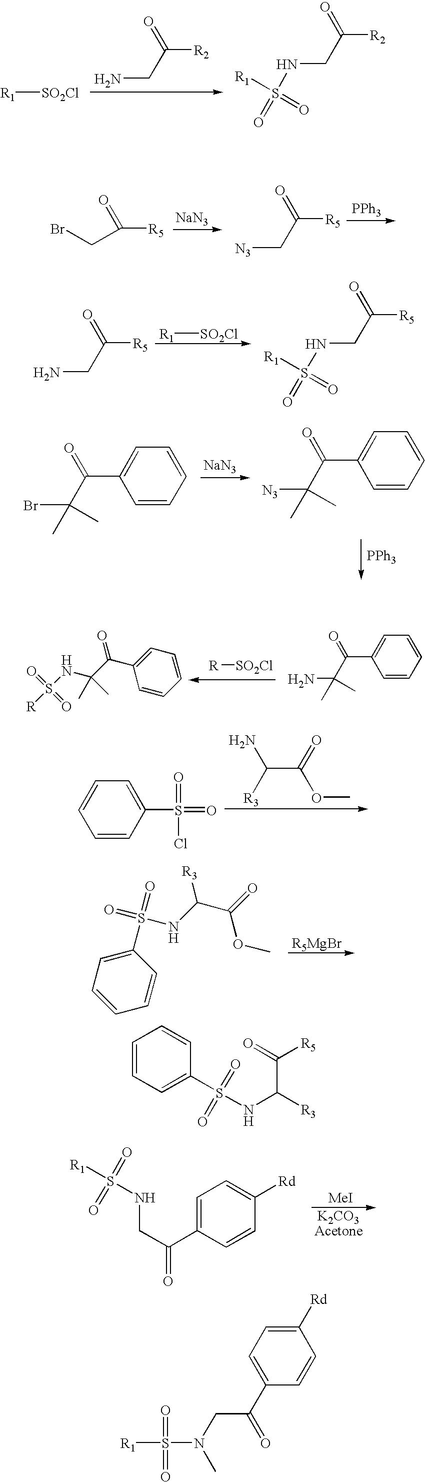

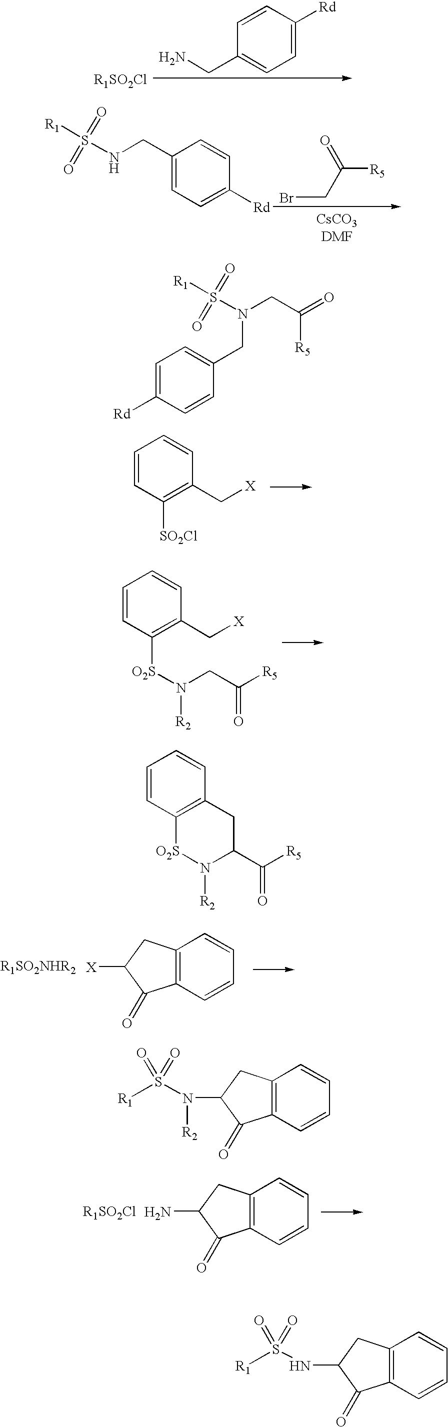

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The term “pharmaceutically acceptable carrier or adjuvant” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

In general, the compounds described herein can be used for treating, controlling, ameliorating, preventing, delaying the onset of, or reducing the risk of developing one or more diseases, disorders, conditions or symptoms mediated by excess or uncontrolled amounts of cortisol and/or other corticosteroids. While not wishing to be bound by any theory, it is believed that the compounds described herein can reduce the levels of cortisol and other corticosteroids (e.g., 11β-hydroxysteroids) by inhibiting the reductase activity of 11β-HSD1. The diseases, disorders, conditions or symptoms mediated by excess or uncontrolled amounts of cortisol and/or other corticosteroids can include diabetes (e.g., type 1 or type 2 diabetes), Syndrome X, hyperglycemia, low glucose tolerance, insulin resistance, obesity, lipid disorders, dyslipidemia, hyperlipidemia, hypertriglyceridemia, hypercholesterolemia, low HDL levels, high LDL levels, atherosclerosis and its sequelae, vascular restenosis, pancreatitis, abdominal obesity, neurodegenerative disease, retinopathy, nephropathy, neuropathy, hypertension, coronary heart disease, stroke, peripheral vascular disease, Cushing's syndrome, glaucoma, osteoperosis, hyperinsulinemia, tuberculosis, psoriasis,cognitive disorders and dementia (e.g., impairment associated with aging and of neuronal dysfunction, e.g., Alzheimer's disease), depression, viral diseases, inflammatory disorders, immune disorders. In some embodiments, the diseases, disorders conditions or symptoms can further include those where insulin resistance is a component. In other embodiments, the compounds described herein can be used for promoting wound healing.

The compounds described herein generally have an inhibition constant IC₅₀ of less than about 500 nM (e.g., less than about 400 nM, less than about 300 nM, less than about 200 nM, less than about 100 nM). Generally, the IC₅₀ ratio for 11-beta-HSD2 to 11-beta-HSD1 of a compound is at least about 100 or greater.

In some embodiments, the compounds described herein can be coadministered with one or more other threapeutic agents. In certain embodiments, the additional agents may be administered separately, as part of a multiple dose regimen, from the compounds of this invention (e.g., sequentially, e.g., on different overlapping schedules with the administration of one or more compounds of formula (I)). Alternatively, those agents may be part of a single dosage form, mixed together with the compounds of this invention in a single composition (e.g., simultaneously or at about the same with one or more compounds of formula (I)). When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen.

Other therapeutic agents can include DP-IV inhibitors; insulin sensitizers (e.g., (i) PPAR agonists and (ii) biguanides); insulin and insulin analogues and mimetics; sulfonylureas and other insulin secretagogues; prandial glucose regulators, alpha.-glucosidase inhibitors; glucagon receptor antagonists; GLP-1, GLP-1 mimetics, and GLP-1 receptor agonists; GIP,GIP mimetics, and GIP receptor agonists; PACAP, PACAP mimetics, and PACAP receptor 3 agonists; cholesterol lowering agents (e.g., (i) HMG-CoA reductase inhibitors, (ii) sequestrants, (iii) nicotinyl alcohol, nicotinic acid and salts thereof, (iv) PPAR.alpha. agonists, (v) PPAR.alpha./.gamma. dual agonists, (vi) inhibitors of cholesterol absorption, (vii) acyl CoA:cholesterol acyltransferase inhibitors, and (viii) anti-oxidants; PPAR.delta. agonists); antiobesity compounds (e.g., sibutramine and orlisat); an ileal bile acid transporter inhibitor; anti-inflammatory agents excluding glucocorticoids (e.g., aspirin); protein tyrosine phosphatase-1B (PTP-1B) inhibitors; agents that suppress hepatic glucose output (e.g., metformin); agents designed to reduce the absorption of glusoce from the intestine (e.g., acarbose); agents designed to treat the complications of prolonged hyperglycemia (e.g., aldose reductase inhibitors); antidiabetic agents (e.g., glusoce phosphatase inhibitors, glucose-6-phosphatase inhibitors, glucagon receptor antagonists, glucose kinase activators, glycogen phosphorylase inhibitors, fructose 1,6 bisphosphatase inhibitors, glutamine:fructose-6-phosphate amidotransferase inhibitors); antihypertensive agents (e.g., blockers (e.g., atenolol, inderal), ACE inhibitors (e.g., lisinopril), calcium agonists (e.g., nifedipine), angiotensin receptor antagonists (e.g., candesartan), a agonists and diuretic agents (e.g., furosemide, benzthiazide)); and haemostasis modulators (e.g., antithrombotics, activators of fibrinolysis and antiplatelet agents (e.g., clopidogrel, aspirin), thrombin antagonists, factor Xa inhibitors, factor VIIa inhibitors, anticoagulants (e.g., heparin and low molecular weight analogues, hirudin), warfarin).

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/Kg to about 1000 mg/Kg, (e.g., from about 0.01 to about 100 mg/kg, from about 0.1 to about 100 mg/Kg, from about 1 to about 100 mg/Kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or administration by injection. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of this invention is useful when the desired treatment involves areas or organs readily accessible by topical application. For application topically to the skin, the composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of this invention may also be topically applied to the lower intestinal tract by rectal suppository formulation or in a suitable enema formulation.

Topically-transdermal patches are also included in this invention. Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

The invention will be further described in the following examples. It should be understood that these examples are for illustrative purposes only and are not to be construed as limiting this invention in any manner.

### EXAMPLES

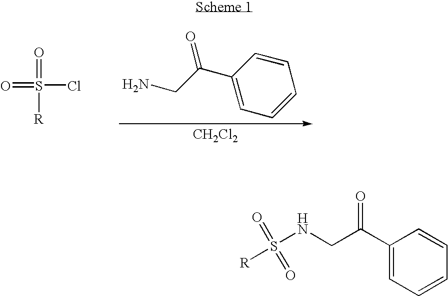

### Example 1A

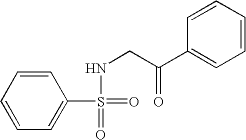

### N-(2-Oxo-2-phenyl-ethyl)-benzenesulfonamide

Step 1A: To a stirred solution of 2-aminoacetophenone HCl salt (10 g, 58.26 mmol) in anhydrous dichloromethane (160 mL) was added Et₃N (32.48 mL, 233 mmol) dropwise. The mixture was cool to 0° C. and benzenesulfonyl chloride (7.48 mL, 58.264 mmol) was injected. After stirring for 5 min, the cooling bath was removed, and the reaction mixture was allowed to warm to room temperature and stir for 2 h. Reaction was complete as determined by TLC. The reaction mixture was then diluted with dichloromethane and washed with water and brine. The organic layer was dried over anhydrous MgSO₄, solvent was evaporated, and the crude mixture was subject to trituration with hexane. Crude product obtained was further purified with flash column chromatography to yield N-(2-Oxo-2-phenyl-ethyl)-benzenesulfonamide in 36.8% yield (5.9 g) as white solid.

1H NMR (400 MHz, DMSO-D6) δ ppm 4.49 (d, J=5.8 Hz, 2 H), 7.49-7.70 (m, 6 H), 7.83-7.90 (m, 2 H), 7.90-7.96 (m, 2 H), 8.07 (t, J=5.7 Hz, 1 H).

HRMS: calcd for C₁₄H₁₃NO₃S+H, 276.06944; found (ESI-FrMS, [M+H]¹⁺), 276.06896.

### Example 1B

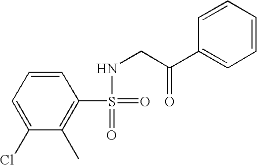

### 3-Chloro-2-methyl-N-(2-oxo-2-phenyl-ethyl)-benzenesulfonamide

Step 1A: Sulfonylation of 2-Aminoacetophenone HCl salt (519 mg, 3.04 mmol) with 3-Chloro-2-methyl-benzenesulfonyl chloride (683 mg, 3.04mmol) was achieved according to a similar procedure described for example 1A using anhydrous Dichloromethane (25 mL) as solvent and Et₃N (1.48 mL, 10.64 mmol) as base.

3-Chloro-2-methyl-N-(2-oxo-2-phenyl-ethyl)-benzenesulfonamide was obtained in 75% yield (560 mg).

1H NMR (400 MHz, DMSO-D6) δ ppm 2.66 (s, 3 H), 4.54 (d, J=5.8 Hz, 2 H), 7.35 (t, J=8.0 Hz, 1 H), 7.49 (t, J=7.7 Hz, 2 H), 7.58-7.73 (m, 2 H), 7.77-7.93 (m, 3 H), 8.41 (t, J=5.8 Hz, 1 H)

HRMS: calcd for C₁₅H₁₄ClNO₃S+H, 324.04612; found (ESI-FFMS, [M+H]¹⁺), 324.0457

### Example 1C

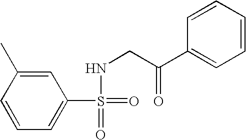

### 3-Methyl-N-(2-oxo-2-phenyl-ethyl)-benzenesulfonamide

Step 1A: Sulfonylation of 2-Aminoacetophenone. HCl (550 mg, 3.22 mmol) with 3-Methyl-benzenesulfonyl chloride (611 mg, 3.22 mmol) was achieved according to a similar procedure described for example 1A using anhydrous Dichloromethane (28 mL) as solvent and Et₃N (1.57 mL, 11.27 mmol). 3-Methyl-N-(2-oxo-2-phenyl-ethyl)-benzenesulfonamide was obtained in 84% yield (680 mg) as white solid.

1H NMR (400 MHz, DMSO-D6) δ ppm 2.37 (s, 3 H), 4.40-4.50 (m, J=5.8 Hz, 2 H), 7.37-7.56 (m, 4 H), 7.59-7.71 (m, 3 H), 7.91 (dd, J=8.3, 1.3 Hz, 2 H), 7.99 (t, J=5.8 Hz, 1 H)

HRMS: calcd for C₁₅H₁₅NO₃S+H, 290.08509; found (ESI-FTMS, [M+H]¹⁺), 290.0841

### Example 1D

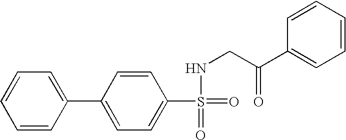

### Biphenyl-4-sulfonic acid (2-oxo-2-phenyl-ethyl)-amide

Step 1A: Sulfonylation of 2-Aminoacetophenone. HCl (419 mg, 2.453 mmol) with Biphenyl-4-sulfonyl chloride (620 mg, 2.453 mmol) was achieved according to a similar procedure described for example 1A using anhydrous Dichloromethane (30 mL) as solvent and Et₃N (1.19 mL, 8.59 mmol). Biphenyl-4-sulfonic acid (2-oxo-2-phenyl-ethyl)-amide was obtained in 81% yield (576 mg).

1H NMR (400 MHz, DMSO-D6) δ ppm 4.51 (t, J=5.6 Hz, 2 H), 7.44 (t, J=6.7 Hz, 1 H), 7.47-7.57 (m, 4 H), 7.65 (t, J=7.3 Hz, 1 H), 7.70-7.78 (m, 2 H), 7.82-7.99 (m, 6 H), 8.11 (s, 1 H)

HRMS: calcd for C₂₀H₁₇NO₃S+H, 352.10074; found (ESI-FTMS, [M+H]¹⁺), 352.0988

### Example 1E

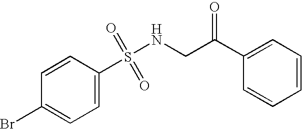

### 4-Bromo-N-(2-oxo-2-phenyl-ethyl)-benzenesulfonamide

Step 1A: Sulfonylation of 2-Aminoacetophenone HCl salt (268.6 mg, 1.565 mmol) with 4-Bromobenzenesulfonyl chloride (400 mg, 1.565 mmol) was achieved according to a similar procedure described for example 1A using anhydrous Dichloromethane (15 mL) as solvent and Et₃N (655 μL, 4.695 mmol) as base.

4-Bromo-N-(2-oxo-2-phenyl-ethyl)-benzenesulfonamide was obtained in 58.6% yield (325 mg) as white solid.

1H NMR (400 MHz, CHLOROFORM-D) δ ppm 4.74 (d, J=4.5 Hz, 2 H), 5.95 (t, J=4.3 Hz, 1 H), 7.74 (t, J=7.8 Hz, 2 H), 7.82-7.94 (m, 3 H), 7.96-8.07 (m, 2 H), 8.11 (dd, J=8.5, 1.4 Hz, 2 H)

HRMS: calcd for C₁₄H₁₂BrNO₃S+H, 353.97995; found (ESI-FTMS, [M+H]¹⁺), 353.9792

### Example 1F

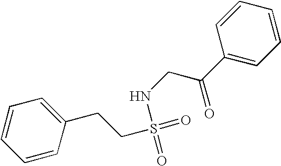

### 2-Phenyl-ethanesulfonic acid (2-oxo-2-phenyl-ethyl)-amide

Step 1A: Sulfonylation of 2-Aminoacetophenone. HCl (285 mg, 1.67 mmol) with 2-Phenyl-ethanesulfonyl chloride (340 mg, 1.67 mmol) was achieved according to a similar procedure described for example 1A using anhydrous Dichloromethane (15 mL) as solvent and Et₃N (700 μL, 5.01 mmol) as base.

2-Phenyl-ethanesulfonic acid (2-oxo-2-phenyl-ethyl)-amide was obtained in 81% yield (409 mg) as white solid.

1H NMR (400 MHz, CHLOROFORM-D) δ ppm 3.07-3.24 (m, 2 H), 3.28-3.42 (m, 2 H), 4.61 (d, J=4.8 Hz, 2 H), 7.11-7.34 (m, 6 H), 7.51 (t, J=7.8 Hz, 2 H), 7.64 (t, J=7.5 Hz, 1 H), 7.93 (d, J=7.6 Hz, 2 H)

HRMS: calcd for C₁₆H₁₇NO₃S+H+, 304.10019; found (ESI-FTMS, [M+H]¹⁺), 304.1003

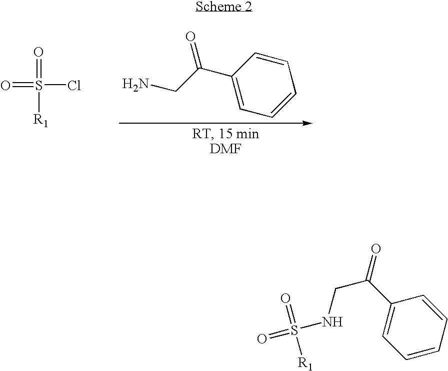

### Example 2A

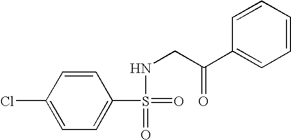

### 4-Chloro-N-(2-oxo-2-phenyl-ethyl)-benzenesulfonamide

Step 2A: To a stirred solution of 2-Aminoacetophenone HCl salt (300 mg, 1.748 mmol) and 4-Chlorobenzenesulfonyl chloride (369 mg, 1.75 mmol) in anhydrous DMF (10 mL) was added Et₃N (0.73 mL, 5.244 mmol) drop wise. Then the reaction mixture was allowed to stir at room temperature for 15 min. Reaction was complete as determined by TLC. The reaction mixture was then poured into cold water, stirred for 20 min. The precipitate thus formed was filtered off, washed with water, air dried under suction and purified by flash chromatography to afford 4-Chloro-N-(2-oxo-2-phenyl-ethyl)-benzenesulfonamide in 50.8% yield (275.4 mg) as white solid.

1H NMR (400 MHz, CHLOROFORM-D) δ ppm 4.33 (d, J=4.5 Hz, 2 H), 5.54 (s, 1 H), 7.24-7.37 (m, 4 H), 7.42-7.51 (m, 1 H), 7.64-7.74 (m, 4 H)

HRMS: calcd for C₁₄H₁₂ClNO₃S+H, 310.03047; found (ESI-FTMS, [M+H]¹⁺), 310.0295

### Example 2B

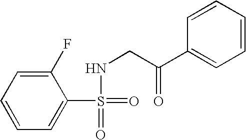

### 2-Fluoro-N-(2-oxo-2-phenyl-ethyl)-benzenesulfonamide

Step 2A: Sulfonylation of 2-Aminoacetophenone. HCl (300 mg, 1.748 mmol) with 2-Fluorobenzenesulfonyl chloride (340.2 mg, 1.748 mmol) was achieved according to a similar procedure described for example 2A using anhydrous DMF (10 mL) as solvent and Et₃N (731 μL, 5.244 mmol) as base. 2-Fluoro-N-(2-oxo-2-phenyl-ethyl)-benzenesulfonamide was obtained in 31% yield (158.9 mg) as white solid.

1H NMR (400 MHz, CHLOROFORM-D) δ ppm 4.37 (d, J=4.5 Hz, 2 H), 5.67 (s, 1 H), 6.98 (dd, J=18.2, 8.1 Hz, 2 H), 7.21-7.29 (m, 2 H), 7.29-7.37 (m, 1 H), 7.37-7.45 (m, 1 H), 7.60-7.67 (m, 2 H), 7.67-7.74 (m, 1 H)

HRMS: calcd for C₁₄H₁₂FNO₃S+H, 294.06002; found (ESI-FTMS, [M+H]¹⁺), 294.0593

### Example 2C

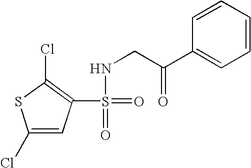

### 2,5-Dichloro-thiophene-3-sulfonic acid (2-oxo-2-phenyl-ethyl)-amide

Step 2A: Sulfonylation of 2-Aminoacetophenone. HCl (300 mg, 1.748 mmol) with 2,5-Dichlorothiophene-3-sulfonyl chloride (439.7 mg, 1.748 mmol) was achieved according to a similar procedure described for example 2A using anhydrous DMF (10 mL) as solvent and Et₃N (731 μL, 5.244 mmol) as base. 2,5-Dichloro-thiophene-3-sulfonic acid (2-oxo-2-phenyl-ethyl)-amide was obtained in 30.5% yield (186.5 mg) as white solid.

1H NMR (400 MHz, CHLOROFORM-D) δ ppm 4.43 (d, J=4.5 Hz, 2 H), 5.79 (t, J=4.2 Hz, 1 H), 7.00 (s, 1 H), 7.26-7.41 (m, 2 H), 7.45-7.57 (m, 1 H), 7.67-7.83 (m, 2 H)

HRMS: calcd for C₁₂H₉C₁₂NO₃S₂+H, 349.94791; found (ESI-FFMS, [M+H]¹⁺), 349.947

### Example 2D

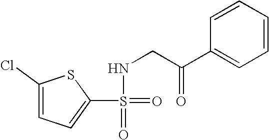

### 5-Chloro-thiophene-2-sulfonic acid (2-oxo-2-phenyl-ethyl)-amide

Step 2A: Sulfonylation of 2-Aminoacetophenone. HCl (300 mg, 1.748 mmol) with 5-Chloro-thiophene-2-sulfonyl chloride (379.5 mg, 1.748 mmol) was achieved according to a similar procedure described for example 2A using anhydrous DMF (10 mL) as solvent and Et₃N (731 μL, 5.244 mmol) as base. 5-Chloro-thiophene-2-sulfonic acid (2-oxo-2-phenyl-ethyl)-amide was obtained in 52.2% yield (287.9 mg) as white solid.

1H NMR (400 MHz, CHLOROFORM-D) δ ppm 4.49 (d, J=4.3 Hz, 2 H), 5.72 (t, J=3.9 Hz, 1 H), 6.83 (d, J=3.8 Hz, 1 H), 7.36 (d, J=4.0 Hz, 1 H), 7.39-7.48 (m, 2 H), 7.52-7.63 (m, 1 H), 7.76-7.87 (m, 2 H)

HRMS: calcd for C₁₂H₁₀ClNO₃S₂+H, 315.98689; found (ESI-FTMS, [M+H]¹⁺), 315.9863

### Example 2E

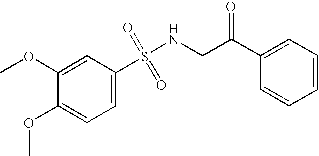

### 3,4-Dimethoxy-N-(2-oxo-2-]2-phenyl-ethyl)-benzenesulfonamide

Step 2A: Sulfonylation of 2-Aminoacetophenone. HCl (300 mg, 1.748 mmol) with 3,4-Dimethoxy-Benzenesulfonyl chloride (413.7 mg, 1.748 mmol) was achieved according to a similar procedure described for example 2A using anhydrous DMF (10 mL) as solvent and Et₃N (731 μL, 5.244 mmol) as base. 3,4-Dimethoxy-N-(2-oxo-2-phenyl-ethyl)-benzenesulfonamide was obtained in 38.7% yield (227.0 mg) as white solid.

1H NMR (400 MHz, CHLOROFORM-D) δ ppm 3.91 (s, 6 H), 4.46 (s, 2 H), 5.65 (s, 1 H), 6.90 (d, J=8.3 Hz, 1 H), 7.34 (d, J=2.0 Hz, 1 H), 7.42-7.54 (m, 3 H), 7.62 (s, 1 H), 7.85 (d, J=7.6 Hz, 2 H)

HRMS: calcd for C₁₆H₁₇NO₅S+H, 336.09057; found (ESI-FTMS, [M+H]¹⁺), 336.0897

### Example 2F

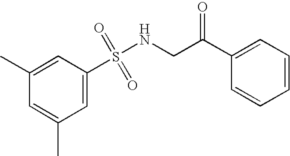

### 3,5-Dimethyl-N-(2-oxo-2-phenyl -ethyl)-benzenesulfonamide

Step 2A: Sulfonylation of 2-Aminoacetophenone. HCl (300 mg, 1.748 mmol) with 3,5-Dimethyl-benzenesulfonyl chloride (357.8 mg, 1.748 mmol) was achieved according to a similar procedure described for example 2A using anhydrous DMF (10 mL) as solvent and Et₃N (731 μL, 5.244 mmol) as base. 3,5-Dimethyl-N-(2-oxo-2-phenyl-ethyl)-benzenesulfonamide was obtained in 34.4% yield (182.6 mg) as white solid.

1H NMR (400 MHz, CHLOROFORM-D) δ ppm 2.35 (s, 6 H), 4.47 (s, 2 H), 5.63 (s, 1 H), 7.17 (s, 1 H), 7.38-7.52 (m, 4 H), 7.62 (s, 1 H), 7.79-7.90 (m, 2 H)

HRMS: calcd for C₁₆H₁₇NO₃S+H, 304.10074; found (ESI-FTMS, [M+H]¹⁺), 304.0999

### Example 2G

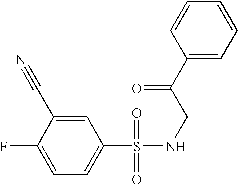

### 3-Cyano-4-fluoro-N-(2-oxo-2-phenyl-ethyl)-benzenesulfonamide

Step 2A: Sulfonylation of 2-Aminoacetophenone. HCl (300 mg, 1.748 mmol) with 3-Cyano-4-Fluoro-benzenesulfonyl chloride (383.4 mg, 1.748 mmol) was achieved according to a similar procedure described for example 2A using anhydrous DMF (10 mL) as solvent and Et₃N (731 μL, 5.244 mmol) as base. 3-Cyano-4-fluoro-N-(2-oxo-2-phenyl-ethyl)-benzenesulfonamide was obtained in 2.7% yield (15.0 mg) as white solid.

1H NMR (400 MHz, CHLOROFORM-D) δ ppm 4.53 (s, 2 H), 5.79 (s, 1 H), 7.37 (t, J=8.5 Hz, 1 H), 7.51 (t, J=7.8 Hz, 2 H), 7.65 (t, J=7.5 Hz, 1 H), 7.87 (d, J=7.3 Hz, 2 H), 8.09-8.18 (m, 1 H), 8.21 (dd, J=5.7, 2.4 Hz, 1 H)

HRMS: calcd for C₁₅H₁₁FN₂O₃S+H, 319.05527; found (ESI-FTMS, [M+H]¹⁺), 319.0543

### Example 2H

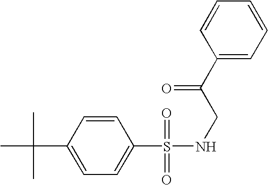

### 4-tert-Butyl-N-(2-oxo-2-phenyl-ethyl)-benzenesulfonamide

Step 2A: Sulfonylation of 2-Aminoacetophenone. HCl (300 mg, 1.748 mmol) with 4-tert-Butyl-benzenesulfonyl chloride (406.8 mg, 1.748 mmol) was achieved according to a similar procedure described for example 2A using anhydrous DMF (10 mL) as solvent and Et₃N (731 μL, 5.244 mmol) as base. 4-tert-Butyl-N-(2-oxo-2-phenyl-ethyl)-benzenesulfonamide was obtained in 22% yield (126.9 mg) as white solid.

1H NMR (400 MHz, CHLOROFORM-D) δ ppm 1.31 (s, 9 H), 4.48 (s, 2 H), 5.65 (s, 1 H), 7.41-7.53 (m, 4 H), 7.56-7.65 (m, 1 H), 7.73-7.96 (m, 4 H)

HRMS: calcd for C₁₈H₂₁NO₃S+H, 332.13204; found (ESI-FTMS, [M+H]¹⁺), 332.1314

### Example 21

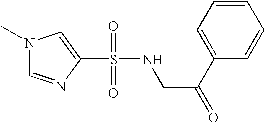

### 1-Methyl-1H-imidazole-4-sulfonic acid (2-oxo-2-phenyl-ethyl)-amide

Step 2A: Sulfonylation of 2-Aminoacetophenone. HCl (300 mg, 1.748 mmol) with 1-Methyl-1H-imidazole-4-sulfonyl chloride (315.7 mg, 1.748 mmol) was achieved according to a similar procedure described for example 2A using anhydrous DMF (10 mL) as solvent and Et₃N (731 μL, 5.244 mmol) as base. 1-Methyl-1H-imidazole-4-sulfonic acid (2-oxo-2-phenyl-ethyl)-amide was obtained in 33% yield (131.8 mg) as off white solid.

1H NMR (400 MHz, DMSO-D6) δ ppm 3.65 (s, 3 H), 4.32-4.48 (m, J=5.1 Hz, 2 H), 7.43-7.55 (m, 2 H), 7.58-7.75 (m, 4 H), 7.83-7.94 (m, 2 H), 8.43 (none, 1 H)

HRMS: calcd for C₁₂H₁₃N₃O₃S+H, 280.07559; found (ESI-FTMS, [M+H]¹⁺), 280.0747.

### Example 2J

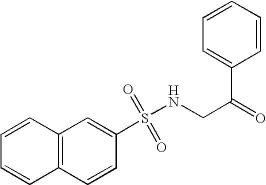

### Naphthalene-2-sulfonic acid (2-oxo-2-phenyl-ethyl)-amide

Step 2A Sulfonylation of 2-Aminoacetophenone. HCl (5.0 g, 29.13 mmol) with Naphtalene-2-sulfonyl chloride (6.6 g, 29.13 mmol) was achieved according to a similar procedure described for example 2A using anhydrous DMF (110 mL) as solvent and Et₃N (12.2 mL, 87.39 mmol) as base. Naphthalene-2-sulfonic acid (2-oxo-2-phenyl-ethyl)-amide was obtained in 28.4% yield (2.686 g) as white solid.

1H NMR (400 MHz, DMSO-D6) δ ppm 4.53 (d, J=5.6 Hz, 2 H), 7.49 (t, J=7.7 Hz, 2 H), 7.59-7.76 (m, 3 H), 7.84-7.95 (m, 3 H), 8.04 (d, J=8.1 Hz, 1 H), 8.13 (t, J=8.2 Hz, 2 H), 8.19 (t, J=5.7 Hz, 1 H), 8.47 (d, J=1.3 Hz, 1 H)

HRMS: calcd for C₁₈H₁₅NO₃S+H, 326.08509; found (ESI-FTMS, [M+H]¹⁺), 326.0842.

### Example 2K

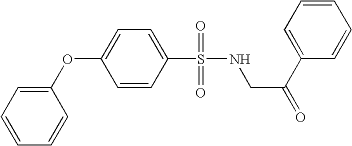

### N-(2-Oxo-2-phenyl-ethyl)-4-phenoxy-benzenesulfonamide

Step 2A: Sulfonylation of 2-Aminoacetophenone. HCl (300 mg, 1.748 mmol) with 4-Phenoxybenzenesulfonyl chloride (469.72 mg, 1.748 mmol) was achieved according to a similar procedure described for example 2A using anhydrous DMF (10 mL) as solvent and Et₃N (731 μL, 5.244 mmol) as base. N-(2-Oxo-2-phenyl-ethyl)-4-phenoxy-benzenesulfonamide was obtained in 45% yield (165.5 mg) as white solid.

1H NMR (400 MHz, CHLOROFORM-D) δ ppm 4.48 (s, 2 H), 5.65 (s, 1 H), 6.94-7.08 (m, 4 H), 7.21 (t, J=7.5 Hz, 1 H), 7.39 (t, J=8.0 Hz, 2 H), 7.49 (t, J=7.8 Hz, 2 H), 7.63 (t, J=7.5 Hz, 1 H), 7.78-7.92 (m, 4 H)

HRMS: calcd for C₂₀H₁₇NO₄S+H, 368.09565; found (ESI-FTMS, [M+H]¹⁺), 368.0947

### Example 2L

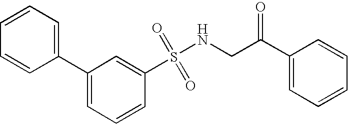

### Biphenyl-3-sulfonic acid (2-oxo-2-phenyl-ethyl)-amide

Step 2A: Sulfonylation of 2-Aminoacetophenone HCl salt (187 mg, 1.09 mmol) with 3-phenylbenzenesulfonyl chloride (275 mg, 1.09 mmol) was achieved according to a similar procedure described for example 2A using anhydrous DMF (10 mL) as solvent and Et₃N (455 μL, 3.264 mmol) as base. Biphenyl-3-sulfonic acid (2-oxo-2-phenyl-ethyl)-amide was obtained in 13% yield (49.3 mg) as white solid.

1H NMR (400 MHz, CHLOROFORM-D) δ ppm 4.52 (s, 2 H), 5.73 (s, 1 H), 7.34-7.53 (m, 5 H), 7.51-7.67 (m, 4 H), 7.77 (d, J=7.8 Hz, 1 H), 7.72-7.93 (m, 3 H), 8.11 (t, J=1.8 Hz, 1 H).

HRMS: calcd for C₂₀H₁₇NO₃S+H, 352.10074; found (ESI-FTMS, [M+H]¹⁺), 352.0997.

### Example 2M

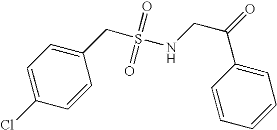

### C-(4-Chloro-phenyl)-N-(2-oxo-2-phenyl-ethyl)-methanesulfonamide

Step 2A: Sulfonylation of 2-Aminoacetophenone. HCl (305 mg, 1.777 mmol) with (4-Chloro-phenyl)-methanesulfonyl chloride (400 mg, 1.777 mmol) was achieved according to a similar procedure described for example 2A using anhydrous DMF (6 mL) as solvent and Et₃N (743 μL, 5.331 mmol) as base, C-(4-Chloro-phenyl)-N-(2-oxo-2-phenyl-ethyl)-methanesulfonamide was obtained in 8% yield (45.54 mg) as white solid.

1H NMR (400 MHz, CHLOROFORM-D) δ ppm 4.15 (s, 2 H), 4.24 (d, J=4.5 Hz, 2 H), 5.18 (s, 1 H), 7.03-7.26 (m, 4 H), 7.34 (t, J=7.7 Hz, 2 H), 7.42-7.54 (m, 1 H), 7.61-7.73 (m, 2 H)

HRMS: calcd for C₁₅H₁₄ClNO₃S+Na, 346.02806; found (ESI-FTMS, [M+Na]¹⁺), 346.0281

### Example 2N

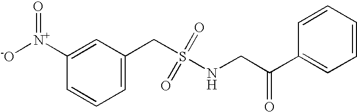

### C-(3-Nitro-phenyl)-N-(2-oxo-2-phenyl-ethyl)-methanesulfonamide

Step 2A Sulfonylation of 2-Aminoacetophenone. HCl (305 mg, 1.777 mmol) with (3-Nitro-phenyl)-methanesulfonyl chlorid (418.7 mg, 1.777 mmol) was achieved according to a similar procedure described for example 2A using anhydrous DMF (6 mL) as solvent and Et₃N (743 μL, 5.331 mmol) as base C-(3-Nitro-phenyl)-N-(2-oxo-2-phenyl-ethyl)-methanesulfonamide was obtained in 14% yield (84.0 mg) as white solid.

1H NMR (400 MHz, CHLOROFORM-D) δ ppm 4.24 (s, 2 H), 4.34 (d, J=4.8 Hz, 2 H), 5.12 (t, J=4.4 Hz, 1 H), 7.26-7.39 (m, 3 H), 7.45 (t, J=7.5 Hz, 1 H), 7.62 (d, J=7.8 Hz, 1 H), 7.65-7.71 (m, 2 H), 7.95-8.04 (m, 1 H), 8.11 (t, J=1.9 Hz, 1 H)

HRMS: calcd for C₁₅H₁₄N₂O₅S+H, 335.07017; found (ESI-FTMS, [M+H]¹⁺), 335.0699.

### Example 20

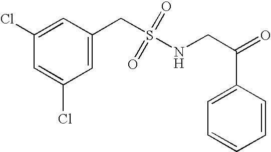

### C-(3,5-Dichloro-phenyl)-N-(2-oxo-2-phenyl-ethyl)-methanesulfonamide

Step 2A Sulfonylation of 2-Aminoacetophenone. HCl (305 mg, 1.777 mmol) with (3,5-Dichloro-phenyl)-methanesulfonyl chloride (461.2 mg, 1.777 mmol) was achieved according to a similar procedure described for example 2A using anhydrous DMF (6 mL) as solvent and Et₃N (743 μL, 5.331 mmol) as base C-(3,5-Dichloro-phenyl)-N-(2-oxo-2-phenyl-ethyl)-methanesulfonamide was obtained in 25% yield (159.1 mg) as white solid.

1H NMR (400 MHz, CHLOROFORM-D) δ ppm 4.30 (s, 2 H), 4.53 (d, J=4.5 Hz, 2 H), 5.36 (t, J=4.4 Hz, 1 H), 7.32-7.40 (m, 3 H), 7.54 (t, J=7.7 Hz, 2 H), 7.67 (d, J=7.6 Hz, 1 H), 7.91 (dd, J=8.5, 1.4 Hz, 2 H)

HRMS: calcd for C₁₅H₁₃C₁₂NO₃S+H, 358.00714; found (ESI-FTMS, [M+H]¹⁺), 358.0064.

### Example 2P

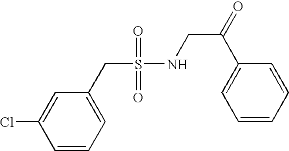

### C-(3-Chloro-phenyl)-N-(2-oxo-2-phenyl-ethyl)-methanesulfonamide

Step2A: Sulfonylation of 2-Aminoacetophenone. HCl (380 mg, 2.21 mmol) 3-Chloro-benzenesulfonyl chloride (495 mg, 2.21 mmol) was achieved according to a similar procedure described for example 2A using anhydrous DMF (15 mL) as solvent and Et₃N (0.92 mL, 6.63 mmol) as base to give C-(3-Chloro-phenyl)-N-(2-oxo-2-phenyl-ethyl)-methanesulfonamide in 77% yield (250 mg).

1H NMR (400 MHz, DMSO-D6) δ ppm 4.47 (s, 2 H), 4.62 (d, J=5.8 Hz, 2 H), 7.36-7.62 (m, 7 H), 7.69 (t, J=7.3 Hz, 1 H), 7.92-8.03 (m, 2 H)

HRMS: calcd for 2 C₁₅H₁₄ClNO₃S+H, 647.08441; found (ESI-FTMS, [2M+H]¹⁺), 647.0841

### Example 2Q

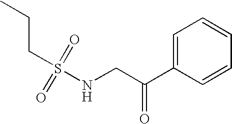

### Propane-1-sulfonic acid (2-oxo-2-phenyl-ethyl)-amide

Step 2A Sulfonylation of 2-Aminoacetophenone. HCl (400 mg, 2.33 mmol) with 1-Propanesulfonyl chloride (332 3 mg, 2.33 mmol) was achieved according to a similar procedure described for example 2A using anhydrous DMF (10 mL) as solvent and Et₃N (974 μL, 6.99 mmol) as base. Propane-1-sulfonic acid (2-oxo-2-phenyl-ethyl)-amide was obtained in 32.5% yield (182.5 mg) as white solid.

1H NMR (400 MHz, CHLOROFORM-D) δ ppm 1.02 (t, J=7.5 Hz, 3 H), 1.79-1.91 (m, 2 H), 2.91-3.04 (m, 2 H), 4.61 (d, J=4.5 Hz, 2 H), 5.23 (s, 1 H), 7.47 (t, J=7.7 Hz, 2 H), 7.54-7.67 (m, 1 H), 7.76-7.95 (m, 2 H)

HRMS: calcd for C₁₁H₁₅NO₃S+H, 242.08509; found (ESI-FTMS, [M+H]¹⁺), 242.0845.

### Example 2R

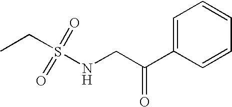

### Ethanesulfonic acid (2-oxo-2-phenyl-ethyl)-amide

Step 2A Sulfonylation of 2-Aminoacetophenone. HCl (400 mg, 2.33 mmol) with Ethanesulfonyl chloride (221 μL, 2.33 mmol) was achieved according to a similar procedure described for example 2A using anhydrous DMF (10 mL) as solvent and Et₃N (974 μL, 6.99 mmol) as base. Ethanesulfonic acid (2-oxo-2-phenyl-ethyl)-amide was obtained in 20% yield (106.0 mg) as off white solid.

1H NMR (400 MHz, CHLOROFORM-D) δ ppm 1.25 (t, J=7.5 Hz, 3 H), 2.92 (q, J=7.3 Hz, 2 H), 4.49 (d, J=4.8 Hz, 2 H), 5.11 (s, 1 H), 7.28-7.39 (m, 2 H), 7.42-7.52 (m, 1 H), 7.77 (dd, J=8.5, 1.1 Hz, 2 H)

HRMS: calcd for C₁₀H₁₃NO₃S+H, 228.06944; found (ESI-FTMS, [M+H]¹⁺), 228.0688;

### Example 2S

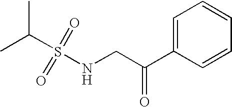

### Propane-2-sulfonic acid (2-oxo-2-phenyl-ethyl)-amide

Step 2A Sulfonylation of 2-Aminoacetophenone. HCl (400 mg, 2.33 mmol) with Isopropylsulfonyl chloride (332.3 mg, 2.33 mmol) was achieved according to a similar procedure described for example 2A using anhydrous DMF (10 mL) as solvent and Et₃N (974 μL, 6.99 mmol) as base. Propane-2-sulfonic acid (2-oxo-2-phenyl-ethyl)-amide was obtained in 4.5% yield (24.9 mg) as light yellow solid.

1H NMR (400 MHz, CHLOROFORM-D) δ ppm 1.21 (d, J=6.8 Hz, 6 H), 2.82-3.16 (m, 1 H), 4.46 (d, J=4.5 Hz, 2 H), 4.98 (s, I H), 7.30 (t, J=7.7 Hz, 2 H), 7.37-7.57 (m, 1 H), 7.73 (d, J=7.8 Hz, 2 H)

HRMS: calcd for C₁₁H₁₅NO₃S+H, 242.08509; found (ESI-FTMS, [M+H]¹⁺), 242.0845.

### Example 2T

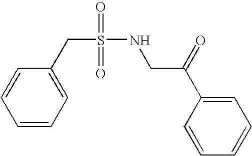

### N-(2-Oxo-2-phenyl-ethyl)-C-phenyl-methanesulfonamide

Step 2A: Sulfonylation of 2-Aminoacetophenone. HCl (360.42 mg, 2.1 mmol) with α-Toluenesulfonyl chloride (400 mg2.1 mmol) was achieved according to a similar procedure described for example 2A using anhydrous DMF (10 mL) as solvent and Et₃N (878 μL, 6.3 mmol) as base. N-(2-Oxo-2-phenyl-ethyl)-C-phenyl-methanesulfonamide was obtained in 36% yield (220.4 mg) as white solid.

1H NMR (400 MHz, CHLOROFORM-D) δ ppm 4.30 (s, 2 H), 4.35 (s, 2 H), 5.34 (s, 1 H), 7.28-7.53 (m, 7 H), 7.62 (t, J=7.5 Hz, 1 H), 7.76-7.82 (m, 2 H)

HRMS: calcd for C₁₅H₁₅NO₃S+Na, 312.06703; found (ESI-FTMS, [M+Na]¹⁺), 312.0667

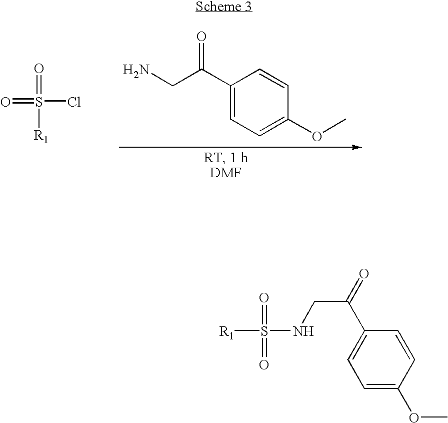

### Example 3A

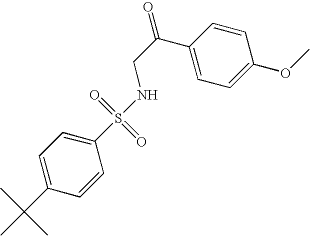

### 4-tert-Butyl-N-[2-(4-methoxy-phenyl)-2-oxo-ethyl]-benzenesulfonamide

Step 3A A solution 2-Amino-4-Methoxy acetophenone.HCl (10.0 g, 49.58 mmol) and 4-tBu-benzenesulfonyl chloride (11.54 g, 49.58 mmol) in anhydrous Dichloromethane (375 mL) was cooled down to 0° C. Et₃N (20.73 mL, 148.74 mmol) was added drop wise then the reaction mixture was allowed to stir at room temperature for 2.5 h. Reaction was complete as determined by TLC. The reaction mixture was then washed with water then by brine. The organic layer was dried over anhydrous MgSO₄, solvent was evaporated, and crude product was triturated. Solid filtered off was subject to flash chromatography to yield 4-tert-Butyl-N-[2-(4-methoxy-phenyl)-2-oxo-ethyl]-benzenesulfonamide in 51.5% yield (9.23 g) as white solid.

1H NMR (400 MHz, DMSO-D6) δ ppm 1.30 (s, 9 H), 3.84 (s, 3 H), 4.37 (d, J=5.3 Hz, 2 H), 7.02 (d, J=9.1 Hz, 2 H), 7.58 (d, J=8.6 Hz, 2 H), 7.77 (d, J=8.6 Hz, 2 H), 7.90 (d, J=8.8 Hz, 3 H)

HRMS: calcd for C₁₉H₂₃NO₄S+H, 362.14260; found (LC-FTMS, [M+H]¹⁺), 362.1443

### Example 3B

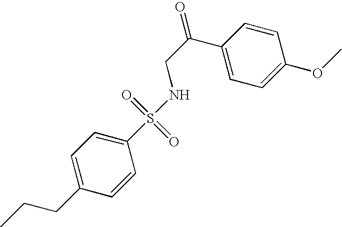

### N-[2-(4-Methoxy-phenyl)-2-oxo-ethyl]-4-propyl-benzenesulfonamide

Step3A Sulfonylation of 2-Amino-4′-methoxy acetophenone. HCl (350 mg, 1.57 mmol) with Propane-1-sulfonyl chloride (224 mg mg, 1.57 mmol) was achieved according to a similar procedure described for example 3A using anhydrous Dichloromethane (15 mL) as solvent and Et₃N (0.66 mL, 4.71 mmol) as base to give N-[2-(4-Methoxy-phenyl)-2-oxo-ethyl]-4-propyl-benzene sulfonamide in 78% yield (140 mg).

1H NMR (400 MHz, DMSO-D6) δ ppm 0.77 (t, J=7.5 Hz, 3 H), 1.38-1.67 (m, 2 H), 2.65-2.93 (m, 2 H), 3.65 (s, 3 H), 4.33 (d, J=5.6 Hz, 2 H), 6.81-6.93 (m, 2 H), 7.12 (t, J=5.7 Hz, 1 H), 7.65-7.85 (m, 2 H)

HRMS: calcd for C₁₂H₁₇NO₄S+H, 272.09565; found (ESI-FTMS, [M+H]¹⁺), 272.0953

### Example 3C

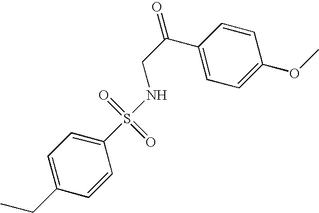

### 4-Ethyl-N-[2-(4-methoxy-phenyl)-2-oxo-ethyl]-benzenesulfonamide

Step3A Sulfonylation of 2-Amino-4′-methoxy acetophenone. HCl (350 mg, 1.57 mmol) with Ethanesulfonyl chloride (202 mg, 1.57 mmol) was achieved according to a similar procedure described for example 3A using anhydrous Dichloromethane (15 mL) as solvent and Et₃N (0.66 mL, 4.71 mmol) as base to give 4-Ethyl-N-[2-(4-methoxy-phenyl)-2-oxo-ethyl]-benzenesulfonamide in 84% yield (125 mg).

1H NMR (400 MHz, DMSO-D6) δ ppm 1.03 (t, J=7.3 Hz, 3 H), 2.84 (q, J=7.3 Hz, 2 H), 3.63 (s, 3 H), 4.32 (d, J=5.8 Hz, 2 H), 6.78-6.96 (m, 2 H), 7.12 (t, J=5.7 Hz, 1 H), 7.75 (d, J=8.8 Hz, 2 H)

HRMS: calcd for C₁₁H₁₅NO₄S+H, 258.08000; found (ESI-FTMS, [M+H]¹⁺), 258.0795

### Example 3D

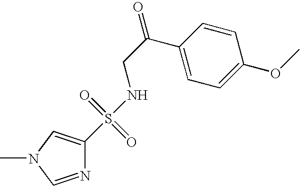

### 1-Methyl-1H-imidazole-4-sulfonic acid [2-(4-methoxy-phenyl)-2-oxo-ethyl]-amide

Step3A: Sulfonylation of 2-Amino-4′-methoxy acetophenone. HCl (350 mg, 1.57 mmol) with 1-Methyl-1H-imidazole-4-sulfonyl chloride (283 mg, 1.57 mmol) was achieved according to a similar procedure described for example 3A using anhydrous Dichloromethane (15 mL) as solvent and Et₃N (0.66 mL, 4:71 mmol) as base to give 1-Methyl-1H-imidazole-4-sulfonic acid [2-(4-methoxy-phenyl)-2-oxo-ethyl]-amide in 86% yield (144 mg).

1H NMR (400 MHz, DMSO-D6) δ ppm 3.72 (s, 3 H), 3.89 (s, 3 H), 4.43 (d, J=5.8 Hz, 2 H), 7.08 (d, J=8.8 Hz, 2 H), 7.64 (t, J=5.7 Hz, 1 H), 7.77 (d, J=5.3 Hz, 2 H), 7.94 (d, J=8.8 Hz, 2 H)

HRMS: calcd for C₁₃H₁₅N₃O₄S+H, 310.08615; found (ESI-FTMS, [M+H]¹⁺), 310.0856

### Example 3E

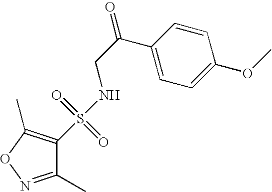

### 3,5-Dimethyl-isoxazole-4-sulfonic acid [2-(4-methoxy-phenyl)-2-oxo-ethyl]-amide

Step3A Sulfonylation of 2-Amino-4′-methoxy acetophenone. HCl (350 mg, 1.57 mmol) 3,5-Dimethyl-isoxazole-4-sulfonyl chloride (306 mg, 1.57 mmol) was achieved according to a similar procedure described for example 3A using anhydrous Dichloromethane (15 mL) as solvent and Et₃N (0.66 mL, 4.71 mmol) as base to give 3,5-Dimethyl-isoxazole-4-sulfonic acid [2-(4-methoxy-phenyl)-2-oxo-ethyl]-amide in 60% yield (123 mg).

1H NMR (400 MHz, DMSO-D6) δ ppm 2.60 (s, 3 H), 2.76 (s, 3 H), 3.97-4.19 (m, 3 H), 4.75 (d, J=5.1 Hz, 2 H), 7.02-7.49 (m, 2 H), 7.97-8.31 (m, 2 H), 8.59 (d, J=4.3 Hz, 1 H)

HRMS: calcd for C₁₄H₁₆N₂O₅S+H, 325.08582; found (ESI-FTMS, [M+H]¹⁺), 325.0856

### Example 3F

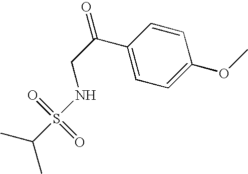

### Propane-2-sulfonic acid [2-(4-methoxy-phenyl)-2-oxo-ethyl]-amide

Step 3A Sulfonylation of 2-Amino-4′-methoxy acetophenone. HCl (707.1 mg, 3.5 mmol) Isopropyl sulfonyl chloride (500 mg mg, 3.5 mmol) was achieved according to a similar procedure described for example 3A using anhydrous Dichloromethane (10 mL) as solvent and Et₃N (1.463 μL, 10.5 mmol) as base to give Propane-2-sulfonic acid [2-(4-methoxy-phenyl)-2-oxo-ethyl]-amide in 20.2% yield (54.8 mg) as light yellow solid.

1H NMR (400 MHz, DMSO-D6) δ ppm 1.26 (d, J=6.8 Hz, 6 H), 3.14-3.27 (m, 1 H), 3.85 (s, 3 H), 4.53 (d, J=5.8 Hz, 2 H), 6.99-7.09 (m, 2 H), 7.31 (s, 1 H), 7.92-8.02 (m, 2H)

HRMS: calcd for C₁₂H₁₇NO₄S+H, 272.09565; found (ESI-FTMS, [M+H]¹⁺), 272.0952

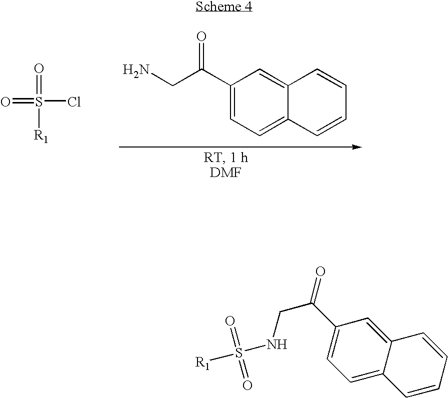

### Example 4A

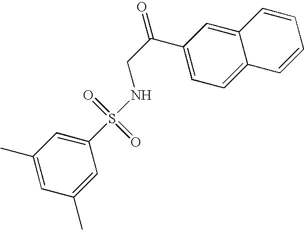

### 3,5-Dimethyl-N-(2-naphthalen-2-yl-2-oxo-ethyl)-benzenesulfonamide

Step 4A To a stirred solution of 2-(2-naphtyl)-2-oxo-1-ethanaminium chloride (400 mg, 1.804 mmol) and 3,5-Dimethyl-benzenesulfonyl chloride (369 mg, 1.804 mmol) in anhydrous DMF (10 mL) was added Et₃N (754 μL, 5.412 mmol) drop wise. Then the reaction mixture was allowed to stir at room temperature for 1 h. Reaction was complete as determined by TLC. The reaction mixture was then diluted with Ethyl acetate and washed with water then by brine. The organic layer was dried over anhydrous MgSO₄, solvent was evaporated, and crude was subject to flash chromatography followed by trituration to yield 3,5-Dimethyl-N-(2-naphthalen-2-yl-2-oxo-ethyl)-benzenesulfonamide in 24% yield (150 mg) as light yellow solid.

1H NMR (400 MHz, DMSO-D6) δ ppm 2.41 (s, 6 H), 4.68 (d, J=5.8 Hz, 2 H), 7.34 (s, 1 H), 7.57 (s, 2 H), 7.75 (d, J=21.2 Hz, 2 H), 8.00 (dd, J=8.7, 1.6 Hz, 1 H), 8.04-8.16 (m, 3 H), 8.21 (d, J=8.1 Hz, 1 H), 8.75 (s, 1 H)

HRMS: calcd for C₂₀H₁₉NO₃S+H, 354.11639; found (ESI-FTMS, [M+H]¹⁺), 354.1157

### Example 4B

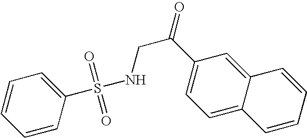

### N-(2-Naphthalen-2-yl-2-oxo-ethyl)-benzenesulfonamide

Step 4A Sulfonylation of 2-(2-naphtyl)-2-oxo-1-ethanaminium chloride (400 mg, 1.804 mmol) with Benzenesulfonyl chloride (230 μL, 1.804 mmol) was achieved according to a similar procedure described for example 4A using anhydrous DMF (10 mL) as solvent and Et₃N (754 μL, 5.412 mmol) as base. N-(2-Naphthalen-2-yl-2-oxo-ethyl)-benzenesulfonamide was obtained in 9% yield (53 mg) as white solid.

1H NMR (400 MHz, DMSO-D6) δ ppm 4.75 (d, J=5.6 Hz, 2 H), 7.57-7.88 (m, 5 H), 7.96-8.07 (m, 3 H), 8.09-8.18 (m, 2 H), 8.17-8.34 (m, 2 H), 8.79 (s, 1 H)

HRMS: calcd for C₁₈H₁₅NO₃S+H, 326.08509; found (ESI-FTMS, [M+H]¹⁺), 326.0847

### Example 4C

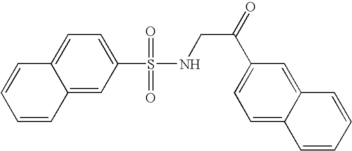

### Naphthalene-2-sulfonic acid (2-naphthalen-2-yl-2-oxo-ethyl)-amide

Step 4A: Sulfonylation of 2-(2-naphtyl)-2-oxo-1-ethanaminium chloride (400 mg, 1.804 mmol) with 2-Naphtalenesulfonyl chloride (409 mg, 1.804 mmol) was achieved according to a similar procedure described for example 4A using anhydrous DMF (10 mL) as solvent and Et₃N (754 μL, 5.412 mmol) as base. Naphthalene-2-sulfonic acid (2-naphthalen-2-yl-2-oxo-ethyl)-amide was obtained in 10.4% yield (70 mg) as white solid.

1H NMR (400 MHz, DMSO-D6) δ ppm 4.64 (d, J=5.8 Hz, 2 H), 7.50-7.71 (m, 4 H), 7.77-8.12 (m, 8 H), 8.20 (t, J=5.7 Hz, I H), 8.46 (d, J=1.5 Hz, 1 H), 8.60 (s, 1 H)

HRMS: calcd for C₂₂H₁₇NO₃S+H, 376.10074; found (ESI-FTMS, [M+H]¹⁺), 376.1005

### Example 4D

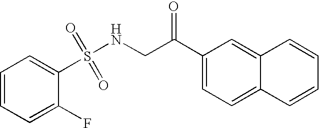

### 2-Fluoro-N-(2-naphthalen-2-yl-2-oxo-ethyl)-benzenesulfonamide

Step 4A: Sulfonylation of 2-(2-naphtyl)-2-oxo-1-ethanaminium chloride (400 mg, 1.804 mmol) with 2-Fluorobenzenesulfonyl chloride (351 mg, 1.804 mmol) was achieved according to a similar procedure described for example 4A using anhydrous DMF (10 mL) as solvent and Et₃N (754 μL, 5.412 mmol) as base. 2-Fluoro-N-(2-naphthalen-2-yl-2-oxo-ethyl)-benzenesulfonamide was obtained in 29.8% yield (200 mg) as light yellow solid.

1H NMR (400 MHz, DMSO-D6) δ ppm 4.78 (d, J=5.6 Hz, 2 H), 7.27-7.45 (m, 2 H), 7.57-7.73 (m, 3 H), 7.77-7.87 (m, 1 H), 7.89 (dd, J=8.6, 1.8 Hz, 1 H), 7.96-8.06 (m, 2 H), 8.12 (d, J=7.8 Hz, 1 H), 8.39 (t, J=5.6 Hz, 1 H), 8.68 (s, 1 H)

HRMS: calcd for C₁₈H₁₄FNO₃S+H, 344.07567; found (ESI-FTMS, [M+H]¹⁺), 344.0753;

### Example 4E

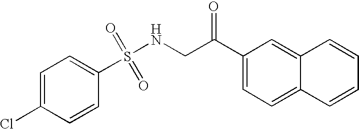

### 4-Chloro-N-(2-naphthalen-2-yl -2-oxo-ethyl)-benzenesulfonamide

Step 4A: Sulfonylation of 2-(2-naphtyl)-2-oxo-1-ethanaminium chloride (400 mg, 1.804 mmol) with 4-Chlorobenzenesulfonyl chloride (380.6 mg, 1.804 mmol) was achieved according to a similar procedure described for example 4A using anhydrous DMF (10 mL) as solvent and Et₃N (754 μL, 5.412 mmol) as base. 4-Chloro-N-(2-naphthalen-2-yl-2-oxo-ethyl)-benzenesulfonamide was obtained in 6.1% yield (40.0 mg) as light yellow solid.

1H NMR (400 MHz, DMSO-D6) δ ppm 4.67 (d, J=5.8 Hz, 2 H), 7.55-7.73 (m, 4 H), 7.82-7.94 (m, 3 H), 7.94-8.04 (m, 2 H), 8.11 (d, J=7.8 Hz, I H), 8.25 (s, 1 H), 8.66 (s, 1 H)

HRMS: calcd for C₁₈H₁₄ClNO₃S+H, 360.04612; found (ESI-FTMS, [M+H]¹⁺), 360.0458

### Example 4F

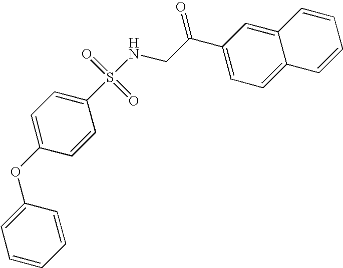

### N-(2-Naphthalen-2-yl-2-oxo-ethyl)-4-phenoxy-benzenesulfonamide

Step 4A: Sulfonylation of 2-(2-naphtyl)-2-oxo-1-ethanaminium chloride (400 mg, 1.804 mmol) with 4-Phenoxybenzenesulfonyl chloride (485.0 mg, 1.804 mmol) was achieved according to a similar procedure described for example 4A using anhydrous DMF (10 mL) as solvent and Et₃N (754 μL, 5.412 mmol) as base. N-(2-Naphthalen-2-yl-2-oxo-ethyl)-4-phenoxy-benzenesulfonamide was obtained in 23.6% yield (178 mg) as light yellow solid.

1H NMR (400 MHz, DMSO-D6) δ ppm 4.50-4.69 (m, J=5.6 Hz, 2 H), 6.94-7.16 (m, 4 H), 7.24 (t, J=7.5 Hz, 1 H), 7.35-7.52 (m, 2 H), 7.55-7.74 (m, 2 H), 7.86 (d, J=8.8 Hz, 2 H), 7.91 (dd, J=8.6, 1.5 Hz, 1 H), 7.95-8.18 (m, 4 H), 8.67 (s, 1 H)

HRMS: calcd for C₂₄H₁₉NO₄S+H, 418.11130; found (ESI-FTMS, [M+H]¹⁺), 418.1111;

### Example 4G

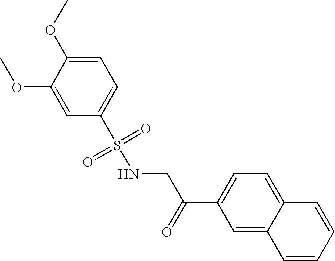

### 3,4-Dimethoxy-N-(2-naphthalen-2-yl-2-oxo-ethyl)-benzenesulfonamide

Step 4A: Sulfonylation of 2-(2-naphtyl)-2-oxo-1-ethanaminiumchloride (400 mg, 1.804 mmol) with 3,4-Dimethoxybenzenesulfonyl chloride (427.0 mg, 1.804 mmol) was achieved according to a similar procedure described for example 4A using anhydrous DMF (10 mL) as solvent and Et₃N (754 μL, 5.412 mmol) as base. 3,4-Dimethoxy-N-(2-naphthalen-2-yl-2-oxo-ethyl)-benzenesulfonamide was obtained in 23.4% yield (163 mg) as pale yellow solid.

1H NMR (400 MHz, DMSO-D6) δ ppm 3.78 (d, J=2.5 Hz, 3 H), 3.82 (d, J=2.3 Hz, 3 H), 4.57 (dd, J=5.7, 2.1 Hz, 2 H), 7.09 (dd, J=8.5, 2.4 Hz, 1 H), 7.30-7.48 (m, 2 H), 7.55-7.76 (m, 2 H), 7.83-8.04 (m, 4 H), 8.11 (d, J=8.1 Hz, 1 H), 8.65 (s, 1 H)

HRMS: calcd for C₂₀H₁₉NO₅S+H, 386.10622; found (ESI-FTMS, [M+H]¹⁺), 386.1065

### Example 4H

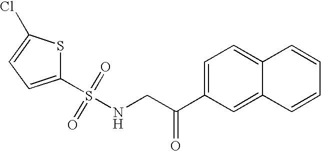

### 5-Chloro-thiophene-2-sulfonic acid (2-naphthalen-2-yl-2-oxo-ethyl)-amide

Step 4A: Sulfonylation of 2-(2-naphtyl)-2-oxo-1-ethanaminiumchloride (400 mg, 1.804 mmol) with 5-Chlorothiophene-2-sulfonyl chloride (391.0 mg, 1.804 mmol) was achieved, according to a similar procedure described for example 4A using anhydrous DMF (10 mL) as solvent and Et₃N (754 μL, 5.412 mmol) as base. 5-Chloro-thiophene-2-sulfonic acid (2-naphthalen-2-yl-2-oxo-ethyl)-amide was obtained in 26.3% yield (174 mg) as off white solid.

1H NMR (400 MHz, DMSO-D6) δ ppm 4.74 (d, J=4.8 Hz, 2 H), 7.24 (d, J=4.0 Hz, 1 H), 7.56 (d, J=4.0 Hz, 1 H), 7.60-7.75 (m, 2 H), 7.89-7.97 (m, 1 H), 8.02 (t, J=8.1 Hz, 2 H), 8.13 (d, J=7.8 Hz, 1 H), 8.54 (s, 1 H), 8.70 (s, 1 H)

HRMS: calcd for C₁₆H₁₂ClNO₃S₂+H, 366.00254; found (ESI-FTMS, [M+H]¹⁺), 366.0023;

### Example 4I

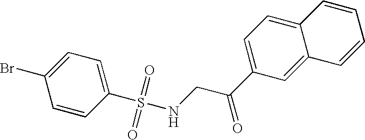

### 4-Bromo-N-(2-naphthalen-2-yl-2-oxo-ethyl)-benzenesulfonamide

Step 4A: Sulfonylation of 2-(2-naphtyl)-2-oxo-1-ethanaminiumchloride (400 mg, 1.804 mmol) with 4-Bromobenzenesulfonyl chloride (391.0 mg, 1.804 mmol) was achieved according to a similar procedure described for example 4A using anhydrous DMF (10 mL) as solvent and Et₃N (754 μL, 5.412 mmol) as base. 4-Bromo-N-(2-naphthalen-2-yl-2-oxo-ethyl)-benzenesulfonamide was obtained in 1.4% yield (9.9 mg) as pale yellow solid.

1H NMR (400 MHz, DMSO-D6) δ ppm 4.48 (d, J=5.8 Hz, 2 H), 7.37-7.54 (m, 3 H), 7.58-7.64 (m, 3 H), 7.68-7.75 (m, 1 H), 7.78-7.86 (m, 2 H), 7.92 (d, J=7.8 Hz, 1 H), 8.08 (t, J=5.7 Hz, 1 H), 8.48 (s, 1 H)

HRMS: calcd for C₁₈H₁₄BrNO₃S+H, 403.99560; found (ESI-FTMS, [M+H]¹⁺), 403.9955

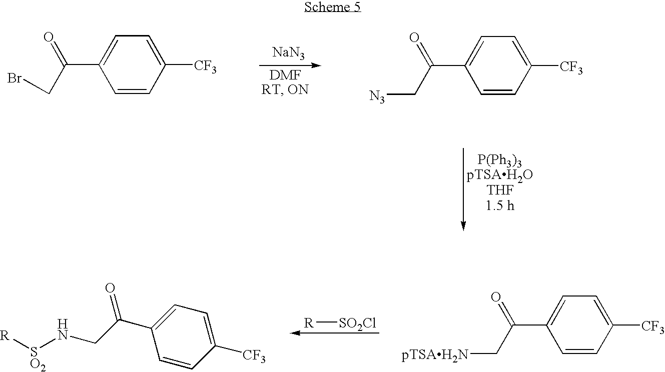

### Example 5A

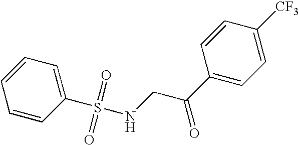

### N-[2-Oxo-2-(4-trifluoromethyl-phenyl)-ethyl]-benzenesulfonamide

Step 5A 2-Bromo-1-(4-trifluoromethyl-phenyl)-ethanone (5 g, 18.73 mmol) dissolved in DMF (50 mL) was added with Sodium azide (1.217 g, 18.73 mmol). The clear orange solution was stirred at room temperature overnight. Reaction was complete as determined by TLC. Then the reaction mixture was partitioned in EtOAc/water, layers were separated then organic layer was washed with brine, dried over anhydrous MgSO₄. Solvent was evaporation followed by flash chromatography yielded 2-Azido-1-(4-trifluoromethyl-phenyl)-ethanone in 29% yield (1.23 g) as orange viscous oil.

1H NMR (400 MHz, DMSO-D6) δ ppm 4.95 (s, 2 H), 8.04 (d, J=69.2 Hz, 4 H)

Step 5B 2-Azido-1-(4-trifluoromethyl-phenyl)-ethanone (1.20 g, 5.24 mmol), Triphenylphosphine (1.374 g, 5.24 mmol) and p-Toluenesulfonicacid monohydrate (3.0 g, 15.72 mmol) were introduced to the reaction flask containing THF (30 mL). The reaction mixture was stirred at room temperature. Clear solution became cloudy in 1 min. Reaction was complete in 1.5 h as determined by TLC. The precipitate formed was filtered off, washed with THF to give 2-Amino-1-(4-trifluoromethyl-phenyl)-ethanone. PTSA salt, in 50.4% yield (990 mg) as white solid.

1H NMR (400 MHz, DMSO-D6) δ ppm 2.28 (s, 3 H), 3.54 (s, 1 H), 4.68 (s, 2 H), 7.11 (d, J=7.8 Hz, 2 H), 7.47 (d, J=8.1 Hz, 2 H), 7.98 (d, J=8.1 Hz, 2 H), 8.21 (d, J=8.1 Hz, 2 H), 8.27 (s, 2 H)

Step 5C To a solution of 2-Amino-1-(4-trifluoromethyl-phenyl)-ethanone. PTSA salt (300 mg, 0.8 mmol) and Benzenesulfonyl chloride (102 μL, 0.8 mmol) in anhydrous DMF (8 mL) was added Triethylamine (342 μL, 2.4 mmol) and the heteregenous mixture was allowed to stir at room temperature for 2.5 h. Reaction was complete as determined by TLC. Then the reaction mixture was poured into cold water (100 mL), stirred for 15 min. Solid was filtered off washed with water, crude product was subject to flash chromatography to afford N-[2-Oxo-2-(4-trifluoromethyl-phenyl)-ethyl]-benzenesulfonamide in 11.4% yield (31.1 mg) as off white solid.

1H NMR (400 MHz, CHLOROFORM-D) δ ppm 4.51 (d, J=4.0 Hz, 2 H), 5.62 (s, 1 H), 7.43-7.63 (m, 3 H), 7.67-7.82 (m, 2 H), 7.94 (dd, J=22.9, 7.7 Hz, 4 H)

HRMS: calcd for C₁₅H₁₂F₃NO₃S+H, 344.05682; found (ESI-FTMS, [M+H]¹⁺), 344.0566.

### Example 5B

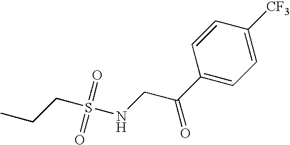

### Propane-1-sulfonic acid [2-oxo-2-(4-trifluoromethyl-phenyl)-ethyl]-amide

Step 5C: Sulfonylation of 2-Amino-1-(4-trifluoromethyl-phenyl)-ethanone. PTSA salt (600 mg, 1.6 mmol) with 1-Propanesulfonyl chloride (228.2 mg, 1.6 mmol) was achieved according to a similar procedure described for example 5A using anhydrous Dichloromethane (14 mL) as solvent and Et₃N (892 μL, 6.4 mmol) as base. Propane-1-sulfonic acid [2-oxo-2-(4-trifluoromethyl-phenyl)-ethyl]-amide was obtained in 11.1% yield (55.2 mg) as white solid.

1H NMR (400 MHz, DMSO-D6) δ ppm 0.98 (t, J=7.5 Hz, 3 H), 1.64-1.85 (m, 2 H), 2.94-3.13 (m, 2 H), 4.66 (t, J=5.8 Hz, 2 H), 7.48 (t, J=5.7 Hz, 1 H), 7.92 (d, J=8.3 Hz, 2 H), 8.18 (d, J=8.1 Hz, 2 H)

HRMS: calcd for C₁₂H₁₄F₃NO₃S+H, 310.07247; found (ESI-FTMS, [M+H]¹⁺), 310.0724.

### Example 5C

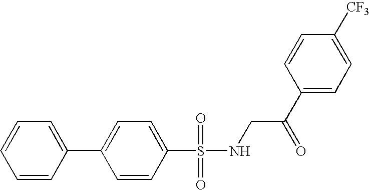

### Biphenyl-4-sulfonic acid [2-oxo-2-(4-trifluoromethyl-phenyl)-ethyl]-amide

Step 5C: Sulfonylation of 2-Amino-1-(4-trifluoromethyl-phenyl)-ethanone. PTSA salt (600 mg, 1.6 mmol) with Biphenyl-4-sulfonyl chloride (404.35 mg, 1.6 mmol) was achieved according to a similar procedure described for example 5A using anhydrous Dichloromethane (15 mL) as solvent and Et₃N (892 μL, 6.4 mmol) as base. Biphenyl-4-sulfonic acid [2-oxo-2-(4-trifluoromethyl-phenyl)-ethyl]-amide was obtained in 18.5% yield (124 mg) as white solid.

1H NMR (400 MHz, DMSO-D6) δ ppm 4.50-4.60 (m, J=5.6 Hz, 2 H), 7.41-7.48 (m, 1 H), 7.52 (t, J=7.5 Hz, 2 H), 7.63-7.78 (m, 2 H), 7.83-7.97 (m, 6 H), 8.11 (d, J=8.1 Hz, 2 H), 8.21 (t, J=5.6 Hz, 1 H);

HRMS: calcd for C₂₁H₁₆F₃NO₃S+H, 420.08812; found (ESI-FTMS, [M+H]¹⁺), 5 420.0879.

### Example 5D

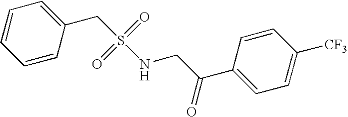

### N-[2-Oxo-2-(4-trifluoromethyl-phenyl)-ethyl]-C-phenyl-methanesulfonamide

Step 5C: Sulfonylation of 2-Amino-1-(4-trifluoromethyl-phenyl)-ethanone.

PTSA salt (600 mg, 1.6 mmol) with α-Toluenesulfonyl chloride (305 mg, 1.6 mmol) was achieved according to a similar procedure described for example 5A using anhydrous Dichloromethane (10 mL) as solvent and Et₃N (892 μL, 6.4 mmol) as base. N-[2-Oxo-2-(4-trifluoromethyl-phenyl)-ethyl]-C-phenyl-methanesulfonamide was obtained in 4.2% yield (24 mg) as white solid.

1H NMR (400 MHz, DMSO-D6) δ ppm 4.44 (s, 2 H), 4.63 (d, J=5.8 Hz, 2 H), 7.30-7.46 (m, 5 H), 7.55 (t, J=5.6 Hz, 1 H), 7.93 (d, J=8.3 Hz, 2 H), 8.15 (d, J=8.3 Hz, 2 H)

HRMS: calcd for C₁₆H₁₄F₃NO₃S+NH₄, 375.09902; found (ESI-FTMS, [M+NH4]¹⁺), 375.0993;

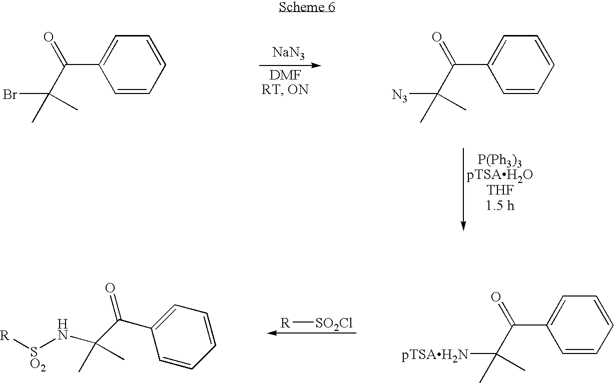

### Example 6A

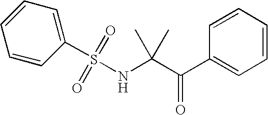

### N-(1,1-Dimethyl-2-oxo-2-phenyl-ethyl)-benzenesulfonamide

Step 6A 2-Bromo-isobutyrophenone (5 g, 22.02 mmol) dissolved in DMF (20 mL) was added with Sodium azide (1.43 g, 22.02 mmol). The clear orange solution was stirred at room temperature for 1 h. Reaction was complete as determined by TLC. Then the reaction mixture was partitioned in EtOAc/water, layers were separated then organic layer was washed with brine, dried over anhydrous MgSO₄. Solvent evaporation yielded 2-Azido-2-methyl-1-phenyl-propan-1-one in quantitative yield (4.166 g) as orange viscous oil. It was immediately carried to the next step.

Step 6B 2-Azido-2-methyl-1-phenyl-propan-1-one (4.166 g, 22.04 mmol), Triphenylphosphine (5.78 g, 22.04 mmol) and p-Toluenesulfonicacid monohydrate (12.58 g, 66.12 mmol) were introduced to a reaction flak containing THF (90 mL). The reaction mixture was stirred at room temperature. Clear solution became cloudy.

Reaction was complete in 25 min as determined by TLC. The precipitate formed was filtered off, washed with THF dried overnight under high vacuum to give 2-Amino-2-methyl-1-phenyl-propan-1-one.PTSA salt, in 47% yield (3.45 g) as white solid.

1H NMR (400 MHz, DMSO-D6) δ ppm 1.71 (s, 6 H), 2.29 (s, 3 H), 7.11 (d, J=7.8 Hz, 2 H), 7.48 (d, J=8.1 Hz, 2 H), 7.58 (t, J=7.7 Hz, 2 H), 7.71 (s, 1 H), 7.91-8.05 (m, 2 H), 8.34 (s, 3 H)

Step 6C To a solution of 2-Amino-2-methyl-1-phenyl-propan-1-one.PTSA salt (600 mg, 1.79 mmol) and Benzene sulfonylchloride (316.15 mg, 1.79 mmol) in anhydrous DMF (20 mL) was added Triethylamine (748 μL, 5.37 mmol) and the heteregenous mixture was allowed to stir at room temperature for 40 min. Reaction was complete as determined by TLC. Then the reaction mixture was poured into cold water (80 mL), stirred for 10 min. Solid product was filtered off, washed with water, purified flash chromatograpy to give N-(1,1-Dimethyl-2-oxo-2-phenyl-ethyl)-benzenesulfonamide in 36% yield (198 mg) as white solid.

1H NMR (400 MHz, DMSO-D6) δ ppm 1.33 (s, 6 H), 7.47 (t, J=7.6 Hz, 2 H), 7.51-7.65 (m, 4 H), 7.67-7.77 (m, 2 H), 8.04-8.15 (m, 2 H), 8.67 (s, 1 H)

HRMS: calcd for C₁₆H₁₇NO₃S+H+, 304.10019; found (ESI-FTMS, [M+H]¹⁺), 304.1004.

### Example 6B

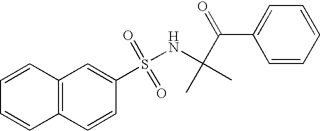

### Naphthalene-2-sulfonic acid (1,1-dimethyl-2-oxo-2-phenyl-ethyl)-amide

Step 6C The sulfonylation of 2-Amino-2-methyl-1-phenyl-propan-1-one.PTSA salt (450 mg, 1.34 mmol) and 2-Naphtalenesulfonyl chloride (303.75 mg, 1.34 mmol) was achieved according to a similar procedure described for example 6A, using anhydrous DMF (10 mL) as solvent and Triethylamine (560 μL, 4.02 mmol) as base. Naphthalene-2-sulfonic acid (1,1-dimethyl-2-oxo-2-phenyl-ethyl)-amide was obtained in 31.27% yield (148.1 mg) as white solid.

1H NMR (400 MHz, DMSO-D6) δ ppm 1.34 (s, 6 H), 7.38-7.59 (m, 3 H), 7.61-7.74 (m, 2 H), 7.78 (dd, J=8.6, 1.8 Hz, 1 H), 8.03 (d, J=8.1 Hz, 1 H), 8.07-8.17 (m, 4 H), 8.31 (d, J=1.5 Hz, 1 H), 8.77 (s, 1 H)

### Example 6C

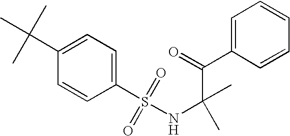

### 4-tert-Butyl-N-(1,1-dimethyl-2-oxo-2-phenyl-ethyl)-benzenesulfonamide

Step 6C The sulfonylation of 2-Amino-2-methyl-1-phenyl-propan-1-one.PTSA salt (450 mg, 1.34 mmol) and 4-tBu-benzenesulfonyl chloride (310.9 mg, 1.34 mmol) was achieved according to a similar procedure described for example 6A, using anhydrous DMF (10 mL) as solvent and Triethylamine (560 μL, 4.02 mmol) as base. 4-tert-Butyl-N-(1,1-dimethyl-2-oxo-2-phenyl-ethyl)-benzenesulfonamide was obtained in 39.3% yield (189.4 mg) as white solid

1H NMR (400 MHz, DMSO-D6) δ ppm 1.30 (s, 9 H), 1.34 (s, 6 H), 7.40-7.71 (m, 7 H), 8.04-8.13 (m, 2 H), 8.57 (s, 1 H)

### Example 6D

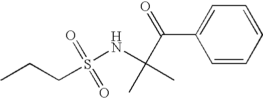

### Propane-1-sulfonic acid (1,1-dimethyl-2-oxo-2-phenyl-ethyl)-amide

Step 6C The sulfonylation of 2-Amino-2-methyl-1-phenyl-propan-1-one.PTSA salt (450 mg, 1.34 mmol) and 4-n-Propylbenzenesulfonyl chloride (293 mg, 1.34 mmol) was achieved according to a similar procedure described for example 6A, using anhydrous DMF (10 mL) as solvent and Triethylamine (560 μL, 4.02 mmol) as base. Propane-1-sulfonic acid (1,1-dimethyl-2-oxo-2-phenyl-ethyl)-amide was obtained in 36.9% yield (171 mg) as white solid.

1H NMR (400 MHz, DMSO-D6) δ ppm 0.88 (t, J=7.3 Hz, 3 H), 1.32 (s, 6 H), 1.49-1.68 (m, 2 H), 2.55-2.68 (m, 2 H), 7.30-7.67 (m, 7 H), 8.04-8.16 (m, 2 H), 8.56 (s, 1 H)

### Example 6E

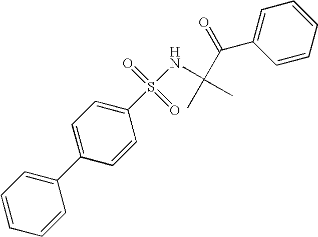

### Biphenyl-4-sulfonic acid (1,1-dimethyl-2-oxo-2-phenyl-ethyl)-amide

Step 6C The sulfonylation of 2-Amino-2-methyl-1-phenyl-propan-1-one.PTSA salt (450 mg, 1.34 mmol) and Biphenyl-4-sulfonyl chloride (338.6 mg, 1.34 mmol) was achieved according to a similar procedure described for example 6A, using anhydrous DMF (10 mL) as solvent and Triethylamine (560 μL, 4.02 mmol) as base. Biphenyl-4-sulfonic acid (1,1-dimethyl-2-oxo-2-phenyl-ethyl)-amide was obtained in 14% yield (71.7 mg) as white solid.

1H NMR (400 MHz, DMSO-D6) δ ppm 1.37 (s, 6 H), 7.50 (dd, J=10.1, 7.8 Hz, 6 H), 7.66-7.91 (m, 6 H), 7.95-8.18 (m, 2 H), 8.71 (s, 1 H).

### Example 6F

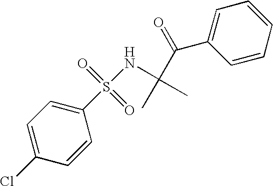

### 4-Chloro-N-(1,1-dimethyl-2-oxo-2-phenyl-ethyl)-benzenesulfonamide

Step 6C The sulfonylation of 2-Amino-2-methyl-1-phenyl-propan-1-one.PTSA salt (450 mg, 1.34 mmol) and 4-Chlorobenzenesulfonyl chloride (282.8 mg, 1.34 mmol) was achieved according to a similar procedure described for example 6A, using anhydrous DMF (10 mL) as solvent and Triethylamine (560 μL, 4.02 mmol) as base. 4-Chloro-N-(1,1-dimethyl-2-oxo-2-phenyl-ethyl)-benzenesulfonamide was obtained in 7.3% yield (33.1 mg) as white solid.

1H NMR (400 MHz, DMSO-D6) δ ppm 1.35 (s, 6 H), 7.47 (t, J=7.6 Hz, 2 H), 7.53-7.66 (m, 3 H), 7.67-7.80 (m, 2 H), 8.02-8.16 (m, 2 H), 8.79 (s, 1 H)

### Example 6G

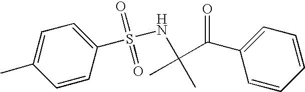

### N-(1,1-Dimethyl-2-oxo-2-phenyl-ethyl)-4-methyl-benzenesulfonamide

Step 6C The sulfonylation of 2-Amino-2-methyl-1-phenyl-propan-1-one.PTSA salt (450 mg, 1.34 mmol) and 4-Chlorobenzenesulfonyl chloride (255.47 mg, 1.34 mmol) was achieved according to a similar procedure described for example 6A, using anhydrous DMF (10 mL) as solvent and Triethylamine (560 μL, 4.02 mmol) as base. N-(1,1-Dimethyl-2-oxo-2-phenyl-ethyl)-4-methyl-benzenesulfonamide was obtained in 46% yield (195.3 mg) as white solid.

1H NMR (400 MHz, DMSO-D6) δ ppm 1.32 (s, 6 H), 2.37 (s, 3 H), 7.35 (d, J=8.1 Hz, 2 H), 7.47 (t, J=7.6 Hz, 2 H), 7.53-7.69 (m, 3 H), 8.01-8.19 (m, 2 H), 8.57 (s, 1 H).

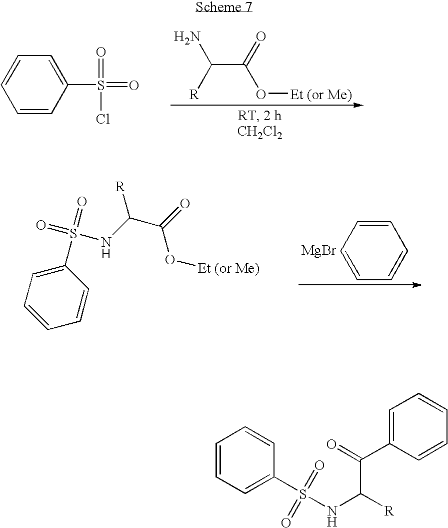

### Example 7A

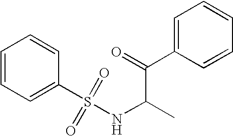

### N-(1-Methyl-2-oxo-2-phenyl-ethyl)-benzenesulfonamide

Step7A: To a stirred solution of DL-Alanine ethyl ester. HCl (3.173 g, 20.656 mmol) and Benzenesulfonyl chloride (3.648 g, 20.656 mmol) in anhydrous Dichloromethane (50 mL), DIEA (8.28 mL, 47.5 mmol) was added drop wise at (−9)° C. The reaction mixture was then allowed to stir at room temperature overnight. The reaction was complete as determined by TLC. The reaction mixture was then diluted with Dichloromethane (100 mL), washed with water, then with brine. Organic layer was dried over MgSO₄, solvent was removed and crude product was purified by flash chromatography to afford 2-Benzenesulfonylamino-propionic acid ethyl ester, in 86.6% yield (4.59 g) as white solid.

1H NMR (400 MHz, CHLOROFORM-D) δ ppm 1.13 (t, J=7.2 Hz, 3 H), 1.39 (d, J=7.1 Hz, 3 H), 3.90-4.05 (m, 3 H), 5.26 (d, J=8.6 Hz, 1 H), 7.45-7.63 (m, 3 H), 7.82-7.90 (m, 2 H)

HRMS: calcd for C₁₁H₁₅NO₄S+H+, 258.07945; found (ESI-FTMS, [M+H]¹⁺), 258.0794.

Step7B: To a dry reaction flask under N₂ was introduced Benzenesulfonylamino-propionic acid ethyl ester (1.0 g, 3.886 mmol). To it was added anhydrous THF (25 mL). Grignard reagent Phenylmagnesium bromide (1.0 M in ether) (7.78 mL. 7.78 mmol) was introduced at 0° C. Then the cooling bath was removed and the reaction mixture was allowed to stir at ambient temperature for 3 h. The reaction was complete as determined by TLC. The reaction was quenched with aq.10% NH₄Cl solution (50 mL) and extracted with EtOAc (100 mL), layers were separated, organic layer was washed with water, then with brine, dried over MgSO₄, solvent was removed and crude product was purified by flash chromatography to afford N-(1-Methyl-2-oxo-2-phenyl-ethyl)-benzenesulfonamide in <1% (7.8 mg) yield as minor product (gummy solid).

1H NMR (400 MHz, CHLOROFORM-D) δ ppm 1.41 (d, J=7.1 Hz, 3 H), 4.86-5.04 (m, 1 H), 5.81 (d, J=7.8 Hz, 1 H), 7.35-7.49 (m, 5 H), 7.59 (t, J=7.5 Hz, 1 H), 7.79 (dd, J=21.9, 7.2 Hz, 4 H)

### Example 7B

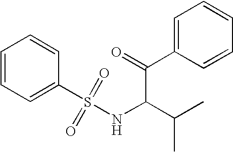

### N-(1-Benzoyl-2-methyl-propyl)-benzenesulfonamide

Step7A: Sulfonylation of DL-Valine methylester. HCl (3.0 g, 17.895 mmol) with Benzenesulfonyl chloride (3.16 g, 17.895 mmol) was achieved according to a similar procedure described for example 7A using anhydrous Dichloromethane (50 mL) as solvent and DIEA (7.17 mL, 41.16 mmol) as base. 2-Benzenesulfonylamino-3-methyl-butyric acid methyl ester was obtained in 71% yield (3.46 g) as white solid.

1H NMR (400 MHz, CHLOROFORM-D) δ ppm 0.92 (dd, J=31.3, 6.8 Hz, 6 H), 1.95-2.12 (m, 1 H), 3.43 (s, 3 H), 3.75 (dd, J=8.5, 5.2 Hz, 1 H), 5.08 (d, J=7.8 Hz, 1 H), 7.45-7.63 (m, 3 H), 7.79-7.89 (m, 2 H)

Step7B: Grignard reaction of 2-Benzenesulfonylamino-3-methyl-butyric acid methyl ester (600 mg, 2.2113 mmol) with Phenylmagnesium bromide (1.0 M in ether) (8.85 mL, 8.844 mmol) was achieved according to a similar procedure described for example 7A using anhydrous THF (10 mL) as solvent. N-(1-Benzoyl-2-methyl-propyl)-benzenesulfonamide was obtained in <1% yield (13.0 mg) as minor product (white solid).

1H NMR (400 MHz, CHLOROFORM-D) δ ppm 0.72 (d, J=6.8 Hz, 3 H), 1.11 (d, J=6.8 Hz, 3 H), 1.96-2.15 (m, 1 H), 4.73 (dd, J=9.3, 3.5 Hz, 1 H), 5.63 (d, J=8.6 Hz, 1 H), 7.28-7.48 (m, 5 H), 7.56 (t, J=7.5 Hz, 1 H), 7.68 (d, J=7.6 Hz, 2 H), 7.77 (d, J=7.3 Hz, 2 H);

HRMS: calcd for C₁₇H₁₉NO₃S+H+, 318.11584; found (ESI-FTMS, [M+H]¹⁺), 318.1161.

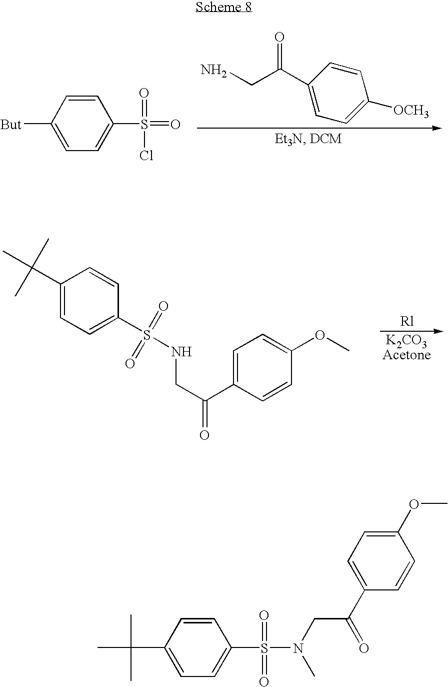

### Example 8A

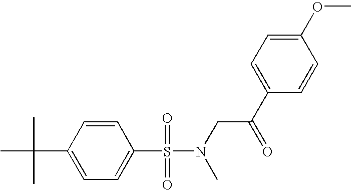

### 4-tert-Butyl-N-[2-(4-methoxy-phenyl)-2-oxo-ethyl]-N-methyl-benzenesulfonamide

Step8A: To a stirred solution of 2-Amino-4-Methoxy acetophenone. HCl (10 g, 49.58 mmol) and 4-tBu-Benzenesulfonyl chloride (11.54 g, 49.58 mmol) in anhydrous Dichloromethane (375 mL), Et₃N (20.73 mL, 148.74 mmol) was added drop wise at 0° C. Then the reaction mixture was allowed to stir at room temperature for 2.5 h. The reaction was complete as determined by TLC. The reaction mixture was washed with water, then with brine. Organic layer was dried over MgSO₄, solvent was removed and crude product was purified by trituration followed by flash chromatography to afford 4-tert-Butyl-N-[2-(4-methoxy-phenyl)-2-oxo-ethyl]-benzenesulfonamide, in 51.5% yield (9.23 g) as white solid.

1H NMR (400 MHz, DMSO-D6) δ ppm 1.30 (s, 9 H), 3.84 (s, 3 H), 4.37 (d, J=5.3 Hz, 2 H), 7.02 (d, J=9.1 Hz, 2 H), 7.58 (d, J=8.6 Hz, 2 H), 7.77 (d, J=8.6 Hz, 2 H), 7.90 (d, J=8.8 Hz, 3 H)

HRMS: calcd for C₁₉H₂₃NO₄S+H+, 362.14206; found (ESI-FTMS, [M+H]¹⁺), 362.1426

Step 8B: To 4-tert-Butyl-N-[2-(4-methoxy-phenyl)-2-oxo-ethyl]-benzenesulfonamide (600 mg, 1.66 mmol) in acetone (5 mL) was added K₂CO₃ powder (344.15 mg, 2.49 mmol) then Iodomethane (5 mL, excess). The reaction mixture was subject to microwave irradiation at 120° C. for 20 min. The reaction was complete as determined by TLC. Solid was filtered off, filtrate was evaporated, residue partitioned in EtOAc/50% sat. brine, layers were separated. Organic layer was dried over MgSO₄, solvent was removed and crude product was purified by flash chromatography to afford 4-tert-Butyl-N-[2-(4-methoxy-phenyl)-2-oxo-ethyl]-N-methyl-benzenesulfonamide in 45.6% yield (284 mg) yield as gummy yellowish solid.

1H NMR (400 MHz, DMSO-D6) δ ppm 1.33 (s, 9 H), 2.74 (s, 3 H), 3.85 (s, 3 H), 4.6 (s, 2 H), 7.06 (d, J=8.8 Hz, 2 H), 7.65 (d, J=8.6 Hz, 2 H), 7.79 (d, J=8.6 Hz, 2 H), 7.97 (d, J=8.8 Hz, 2 H)

HRMS: calcd for C₂₀H₂₅NO₄S 30 H+, 376.15771; found (ESI-FTMS, [M+H]¹⁺), 376.1588

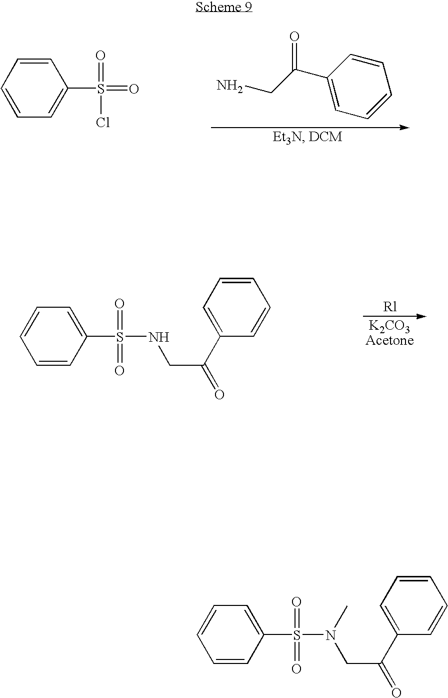

### Example 9A

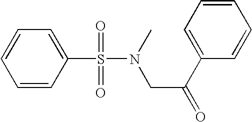

### N-Methyl-N-(2-oxo-2-phenyl-ethyl)-benzenesulfonamide

Step 9A: To a stirred solution of 2-Amino-acetophenone. HCl (5 g, 29.13 mmol) and Benzenesulfonyl chloride (5.15 g, 29.13 mmol) in anhydrous Dichloromethane (150 mL), Et₃N (12.2 mL, 87.39 mmol) was added drop wise at 0° C. Then the reaction mixture was allowed to stir at room temperature for 2.5 h. The reaction was complete as determined by TLC. The reaction mixture was washed with water, then with brine. Organic layer was dried over MgSO₄, solvent was removed and crude product was purified by trituration followed by flash chromatography to afford N-(2-Oxo-2-phenyl-ethyl)-benzenesulfonamide in 57.8% yield 4.63 g) as white solid.

1H NMR (400 MHz, DMSO-D6) δ ppm 4.47 (d, J=5.8 Hz, 2 H), 7.44-7.73 (m, 6 H), 7.80-7.96 (m, 4 H), 8.07 (t, J=5.8 Hz, 1 H)

Step 9B: To N-(2-Oxo-2-phenyl-ethyl)-benzenesulfonamide (300 mg, 1.091 mmol) in acetone (2 mL) was added K₂CO₃ powder (150 mg, 1.091 mmol) then Iodomethane (3.5 mL, excess). The reaction mixture was subject to microwave irradiation at 80° C. for 1 h. The reaction was complete as determined by TLC. Solid was filtered off, filtrate was evaporated, residue partitioned in EtOAc/50% sat. brine, layers were separated. Organic layer was dried over MgSO4, solvent was removed and crude product was purified by flash chromatography to afford N-Methyl-N-(2-oxo-2-phenyl-ethyl)-benzenesulfonamide in 51% yield (160 mg) as white solid.

1H NMR (400 MHz, DMSO-D6) δ ppm 2.78 (s, 3 H), 4.77 (s, 2 H), 7.55 (t, J=7.7 Hz, 2 H), 7.59-7.75 (m, 4 H), 7.82-7.91 (m, 2 H), 7.92-8.03 (m, 2 H)

HRMS: calcd for C₁₅H₁₅NO₃S+H+, 290.08454; found (LC-FTMS, [M+H]¹⁺), 290.0854;

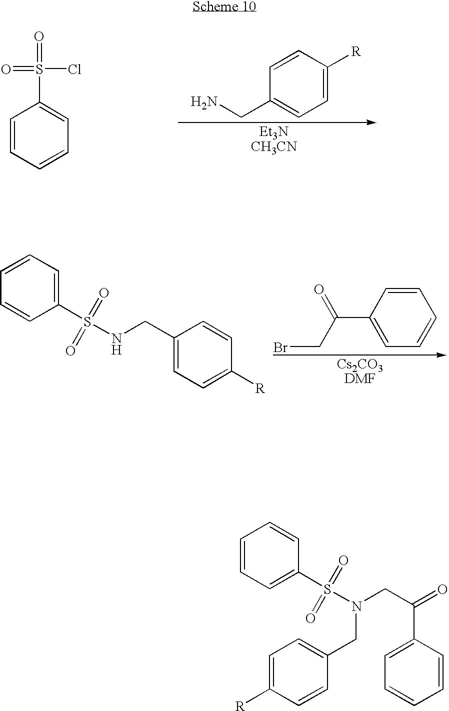

### Example 10A

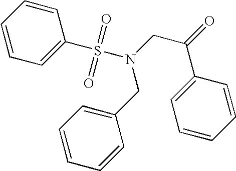

### N-Benzyl-N-(2-oxo-2-phenyl-ethyl)-benzenesulfonamide

Step 10A: To a stirred solution of Benzenesulfonyl chloride (3.0 g, 16.99 mmol) in CH₃CN (50 mL) was added Benzylamine (1.82 g, 16.99 mmol). Initially clear solution became cloudy. Then Et₃N (4.74 mL, 33.97 mmol) was introduced to the reaction mixture, and it was allowed to stir at room temperature for 4 h. Reaction was complete as determined by TLC. Solvent was evaporated and residue was partitioned in Dichloromethane/H₂O. Layers were separated. Organic layer was washed with brine, dried over anhydrous MgSO₄. Solvent evaporation followed by purification by flash chromatography afforded N-Benzyl-benzenesulfonamide in 81% yield (3.4 g) as white solid.

1H NMR (400 MHz, DMSO-D6) δ ppm 3.98 (d, J=6.1 Hz, 2 H), 7.09-7.36 (m, 5 H), 7.49-7.68 (m, 3 H), 7.74-7.85 (m, 2 H), 8.17 (t, J=6.1 Hz, 1 H)

Step 10B: N-Benzyl-benzenesulfonamide (300 mg, 1.213 mmol) dissolved in DMF (8 mL), was added with 2-Bromoacetophenone (265.5 mg, 1.334 mmol) and Cesium carbonate (434.6 mg, 1.334 mmol). The reaction mixture was stirred at ambient temperature for 2 h. Reaction was complete as determined by TLC. Then the reaction mixture was partitioned in Dichloromethane/H₂O. Layers were separated. Organic layer was washed with brine, dried over anhydrous MgSO₄. Solvent evaporation gave crude product, which was subject to flash chromatography to yield in 40.4% yield (179 mg) as pale yellow solid.

1H NMR (400 MHz, DMSO-D6) δ ppm 4.45 (s, 2 H), 4.76 (s, 2 H), 7.10-7.21 (m, 2 H), 7.20-7.28 (m, 3 H), 7.54-7.74 (m, 6 H), 7.87 (dd, J=9.5, 8.0 Hz, 4 H)

HRMS: calcd for C₂₁H₁₉NO₃S+H+, 366.11584; found (ESI-FTMS, [M+H]¹⁺), 366.116;

### Example 10B

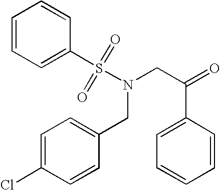

### N-(4-Chloro-benzyl)-N-(2-oxo-2-phenyl-ethyl)-benzenesulfonamide

Step 10A: Sulfonylation of Benzenesulfonyl chloride (830 mg, 4.72 mmol), with 4-Chlorobenzyl amine (670 mg, 4.72 mmol) was achieved according to a similar procedure described for example 10A using anhydrous Acetonitrile (10 mL) as solvent and Et₃N (1.31 mL, 9.44 mmol) as base. N-(4-Chloro-benzyl)-benzenesulfonamide was obtained in 75% yield (799 mg) as white solid.

1H NMR (400 MHz, DMSO-D6) δ ppm 4.13 (s, 2 H), 7.31-7.60 (m, 4 H), 7.58-7.86 (m, 3 H), 7.85-8.09 (m, 2 H), 8.37 (s, 1 H)

Step 10 B: N-Alkylation of N-(4-Chloro-benzyl)-benzenesulfonamide (292 mg, 1.03 mmol) with 2-Bromoacetophenone (226 mg, 1.13 mmol) was achieved according to a similar procedure described for example 10A using anhydrous DMF (8 mL) as solvent and Cesium carbonate (368 mg, 1.13 mmol) as base. N-(4-Chloro-benzyl)-N-(2-oxo-2-phenyl-ethyl)-benzenesulfonamide was obtained in 75% yield (310 mg) as light yellow solid.

1H NMR (400 MHz, DMSO-D6) δ ppm 4.44 (s, 2 H), 4.82 (s, 2 H), 7.20-7.37 (m, 4 H), 7.49 (t, J=7.8 Hz, 2 H), 7.55-7.75 (m, 4 H), 7.81-7.95 (m, 4 H)

HRMS: calcd for C₂₁H₁₈ClNO₃S+H+, 400.07687; found (ESI-FTMS, [M+H]¹⁺), 400.0764;

### Example 10C

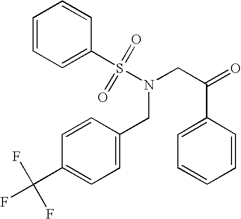

### N-(2-Oxo-2-phenyl-ethyl)-N-(4-trifluoromethyl-benzyl)-benzenesulfonamide

Step 10A: Sulfonylation of Benzenesulfonyl chloride (830 mg, 4.72 mmol), with 4-Trifluoromethyl-benzylamine (830 mg, 4.72 mmol) was achieved according to a similar procedure described for example 10A using anhydrous Acetonitrile (10 mL) as solvent and Et₃N (1.31 mL, 9.44 mmol) as base. N-(4-Trifluoromethyl-benzyl)-benzenesulfonamide was obtained in 64% yield (950 mg).

1H NMR (400 MHz, DMSO-D6) δ ppm 4.10 (s, 2 H), 7.46 (d, J=7.8 Hz, 2 H), 7.52-7.69 (m, 5 H), 7.73-7.86 (m, 2 H), 8.32 (s, 1 H).

Step 10B: N-Alkylation of N-(4-Trifluoromethyl-benzyl)-benzenesulfonamide (560 mg, 1.78 mmol) with 2-Bromoacetophenone (389 mg, 1.96 mmol) was achieved according to a similar procedure described for example 10A using anhydrous DMF (8 mL) as solvent and Cesium carbonate (639 mg, 1.96 mmol) as base. N-(2-Oxo-2-phenyl-ethyl)-N-(4-trifluoromethyl-benzyl)-benzenesulfonamide was obtained in 87% yield (669 mg) as light yellow solid.

1H NMR (400 MHz, DMSO-D6) δ ppm 4.63 (s, 2 H), 4.96 (s, 2 H), 7.51-7.62 (m, 4 H), 7.63-7.81 (m, 6 H), 7.95 (d, J=7.6 Hz, 4 H)

HRMS: calcd for C₂₂H₁₈F₃NO₃S+H+, 434.10322; found (ESI-FTMS, [M+H]¹⁺), 434.1037.

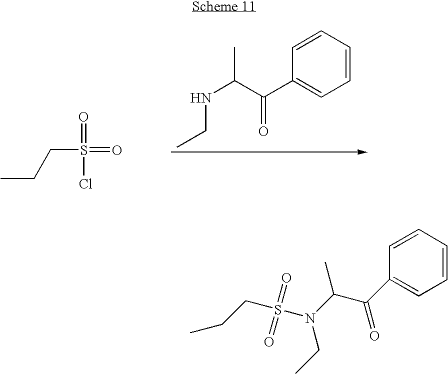

### Example 11A

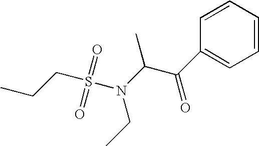

### Propane-1-sulfonic acid ethyl-(1-methyl-2-oxo-2-phenyl-ethyl)-amide

Step 11A: To a stirred solution of 2-(Ethylamino) propiophenone.HCL (600 mg, 2.807 mmol) in Dichloromethane (40 mL) was added NaHCO₃ (2.807 g in 20 mL H₂O). Then 1-Propanesulfonyl chloride (284 μL, 2.526 mmol) was introduced to the reaction mixture. Then the reaction mixture was allowed to stir at room temperature overnight. Reaction was monitored by TLC. Layers were separated. Organic layer was washed with brine, dried over anhydrous MgSO₄. Solvent evaporation gave crude product which was subject to flash chromatography to yield Propane-1-sulfonic acid ethyl-(1-methyl-2-oxo-2-phenyl-ethyl)-amide in <10% yield (26.1 mg) as colorless liquid.

1H NMR (400 MHz, CHLOROFORM-D) δ ppm 1.00 (t, J=7.5 Hz, 3 H), 1.17 (t, J=7.1 Hz, 3 H), 1.44-1.59 (m, J=7.3 Hz, 3 H), 1.73-1.92 (m, 2 H), 2.72-3.01 (m, 2 H), 3.29-3.47 (m, 2 H), 5.56 (q, J=7.3 Hz, 1 H), 7.49 (t, J=7.6 Hz, 2 H), 7.60 (t, J=7.5 Hz, 1 H), 7.93-8.07 (m, 2 H).

HRMS: calcd for C₁₄H₂₁NO₃S+H+, 284.13149; found (ESI-FTMS, [M+H]¹⁺), 284.1314;

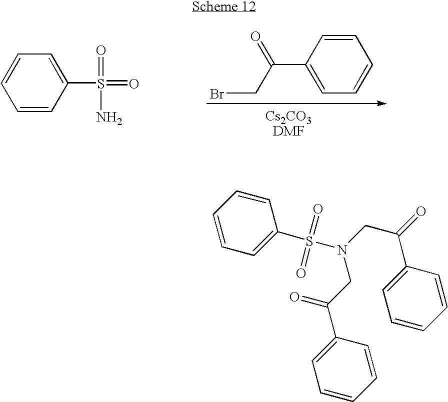

### Example 12A

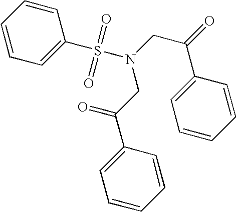

### N,N-Bis-(2-oxo-2-phenyl-ethyl)-benzenesulfonamide

Step 12A: To a stirred solution of Benzenesulfonamide (303 mg, 1.93 mmol) and 2-Bromo-1-phenyl-ethanone (384 mg, 1.93 mmol) in DMF (8 mL), was added Cesium carbonate (692 mg, 2.12 mmol). The resulting heterogeneous mixture was stirred at room temperature for 3 h. Reaction was complete as determined by TLC. Reaction mixture was partitioned in EtOAc/H₂O. Layers were separated; organic layer was washed with brine and dried over anhydrous MgSO₄. Solvent evaporation gave crude product. Purification was achieved by flash chromatography to yield N,N-Bis-(2-oxo-2-phenyl-ethyl)-benzenesulfonamide in 70% yield (531 mg) as light yellow solid

1H NMR (400 MHz, DMSO-D6) δ ppm 5.04 (s, 4 H), 7.51-7.74 (m, 9 H), 7.91-8.02 (m, 6H)

HRMS: calcd for C₂₂H₁₉NO₄S+H+, 394.11076; found (ESI-FTMS, [M+H]¹⁺), 394.1104

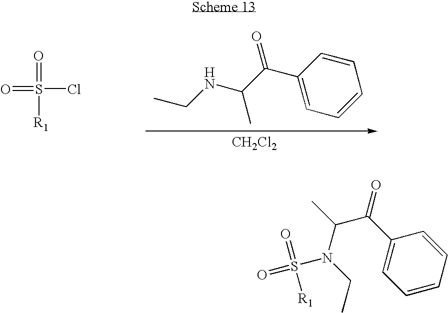

### Example 13A

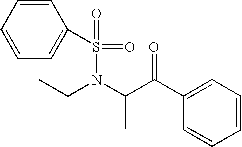

### N-Ethyl-N-(1-methyl-2-oxo-2-phenyl-ethyl)-benzenesulfonamide

Step 13A: A solution of 2-(Ethylamino) propiophenone (500 mg, 2.34 mmol), Benzenesulfonyl chloride (413 mg, 2.34 mmol), Et₃N (0.98 mL, 7.02 mmol) in anhydrous Dichloromethane (5 mL) was stirred at RT for 3 h.

The reaction mixture was then subject to flash chromatography to yield N-Ethyl-N-(1-methyl-2-oxo-2-phenyl-ethyl)-benzenesulfonamide in 42% yield (310 mg) as white solid.

1H NMR (400 MHz, CHLOROFORM-D) δ ppm 1.06 (t, J=7.1 Hz, 3 H), 1.25 (d, J=7.1 Hz, 3 H), 3.14-3.42 (m, 2 H), 5.63 (q, J=7.0 Hz, I H), 7.39-7.63 (m, 6 H), 7.77-7.85 (m, 2H), 8.02-8.11 (m, 2H)

HRMS: calcd for C₁₇H₁₉NO₃S+H+, 318.11584; found (ESI-FTMS, [M+H]¹⁺), 318.1168

### Example 13B

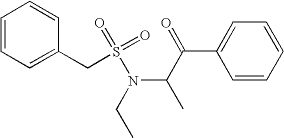

### N-Ethyl-N-(1-methyl-2-oxo-2-phenyl-ethyl)-C-phenyl-methanesulfonamide

Step 13A: Sulfonylation of 2-(Ethylamino) propiophenone (500 mg, 2.34 mmol) with Phenyl-methanesulfonyl chloride (401 mg, 2.11 mmol) was achieved according to similar procedure described for example 13A using Et₃N (0.98 mL, 7.02 mmol) as base and anhydrous Dichloromethane (5 mL) as solvent. N-Ethyl-N-(1-methyl-2-oxo-2-phenyl-ethyl)-C-phenyl-methanesulfonamide was obtained in 30% yield (57 mg) as white low melting solid.

1H NMR (400 MHz, CHLOROFORM-D) δ ppm 1.17 (t, J=7.2 Hz, 3 H), 1.39 (d, J=7.1 Hz, 3 H), 3.28-3.42 (m, 2 H), 4.06-4.28 (m, 2 H), 5.36 (q, J=7.1 Hz, I H), 7.27-7.63 (m, 5 H), 7.47 (t, J=7.6 Hz, 2 H), 7.58 (t, J=7.3 Hz, 1 H), 7.75-8.00 (m, 2 H) HRMS: calcd for C₁₈H₂₁NO₃S+H+, 332.13149; found (ESI-FTMS, [M+H]¹⁺), 332.1324.

### Example 13C

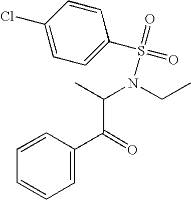

### 4-Chloro-N-ethyl-N-(1-methyl-2-oxo-2-phenyl-ethyl)-benzenesulfonamide

Step 13A: Sulfonylation of 2-(Ethylamino) propiophenone (500 mg, 2.34 mmol) with 4-Chlorobenzenesulfonyl chloride (444 mg, 2.11 mmol), was achieved according to similar procedure described for example 13A using Et₃N (0.98 mL, 7.02 mmol) as base and anhydrous Dichloromethane (5 mL) as solvent. 4-Chloro-N-ethyl-N-(l-methyl-2-oxo-2-phenyl-ethyl)-benzenesulfonamide was obtained as yellowish solid (318 mg).

1H NMR (400 MHz, CHLOROFORM-D) □ ppm 1.09 (t, J=7.2 Hz, 3 H), 1.31 (d, J=6.8 Hz, 3 H), 3.28 (q, 2 H), 5.61 (q, J=6.9 Hz, 1 H), 7.41 (d, J=8.6 Hz, 2 H), 7.48 (t, J=7.8 Hz, 2 H), 7.60 (t, J=7.3 Hz, 1 H), 7.70 (d, J=8.3 Hz, 2 H), 8.01 (d, J=7.6 Hz, 2 H)

HRMS: calcd for C₁₇H₈₁ClNO₃S+H+, 352.07687; found (ESI-FTMS, [M+H]¹⁺), 352.077;

### Example 13D

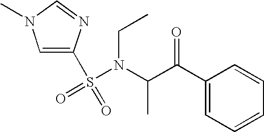

### 1-Methyl-1H-imidazole-4-sulfonic acid ethyl-(1-methyl-2-oxo-2-phenyl-ethyl)-amide

Step 13A: Sulfonylation of 2-(Ethylamino) propiophenone (500 mg, 2.34 mmol) with 1-Methyl-1H-imidazole-4-sulfonyl chloride (380 mg, 2.11 mmol) was achieved according to similar procedure described for example 13A using Et₃N (0.98 mL, 7.02 mmol) as base, anhydrous Dichloromethane (5 mL) as solvent and catalytic amount of DMAP. 1-Methyl-1H-imidazole-4-sulfonic acid ethyl-(1-methyl-2-oxo-2-phenyl-ethyl)-amide was obtained in 91% yield (825 mg) as white solid.

1H NMR (400 MHz, DMSO-D6) δ ppm 0.90 (t, J=7.1 Hz, 3 H), 1.19 (d, J=6.8 Hz, 3 H), 2.98-3.29 (m, 2 H), 3.69 (s, 3 H), 5.52 (q, J=6.7 Hz, 1 H), 7.54 (t, J=7.7 Hz, 2 H), 7.62-7.68 (m, J=7.5, 7.5 Hz, 1 H), 7.79 (s, 2 H), 8.04 (d, J=7.3 Hz, 2 H).

### Example 14

Additional 11βHSD1 compounds are provided in Table 1.

TABLE 1

| | Chemical Names |
|---|---|
| 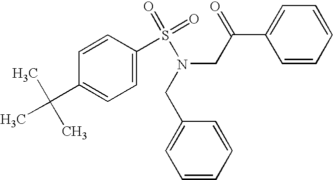 | N-benzyl-4-tert-butyl-N-(2-oxo-2- phenylethyl)benzenesulfonamide |
| 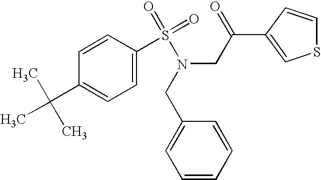 | N-benzyl-4-tert-butyl-N-[2-oxo-2-(3- thienyl)ethyl]benzenesulfonamide |
| 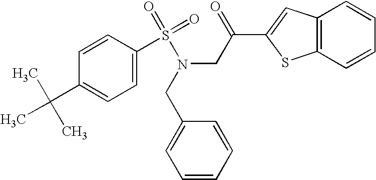 | N-[2-(1-benzothien-2-yl)-2-oxoethyl]-N-benzyl-4-tert- butylbenzenesulfonamide |
| 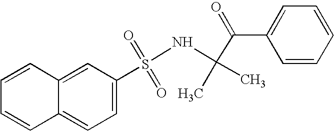 | N-(1,1-dimethyl-2-oxo-2-phenylethyl)naphthalene-2- sulfonamide |
| 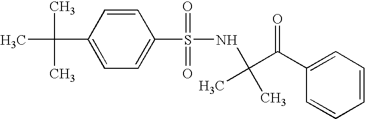 | 4-tert-butyl-N-(1,1-dimethyl-2-oxo-2- phenylethyl)benzenesulfonamide |
| 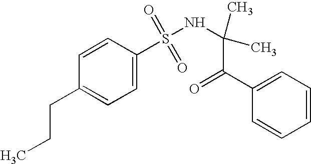 | N-(1,1-dimethyl-2-oxo-2-phenylethyl)-4- propylbenzenesulfonamide |
| 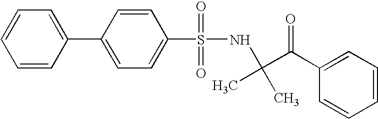 | N-(1,1-dimethyl-2-oxo-2-phenylethyl)biphenyl-4- sulfonamide |
| 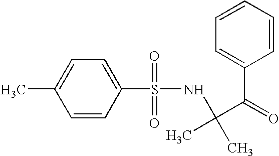 | N-(1,1-dimethyl-2-oxo-2-phenylethyl)-4-methylbenzenesulfonamide |
| 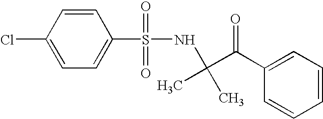 | 4-chloro-N-(1,1-dimethyl-2-oxo-2- phenylethyl)benzenesulfonamide |
| 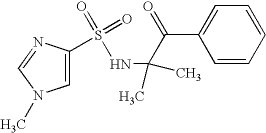 | N-(1,1-dimethyl-2-oxo-2-phenylethyl)-1-methyl-1H- imidazole-4-sulfonamide |
| 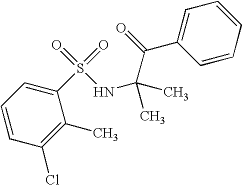 | 3-chloro-N-(1,1-dimethyl-2-oxo-2-phenylethyl)-2- methylbenzenesulfonamide |
| 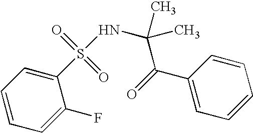 | N-(1,1-dimethyl-2-oxo-2-phenylethyl)-2- fluorobenzenesulfonamide |
| 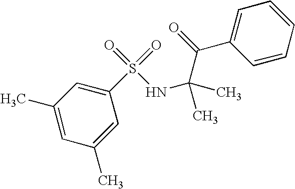 | N-(1,1-dimethyl-2-oxo-2-phenylethyl)-3,5- dimethylbenzenesulfonamide |
| 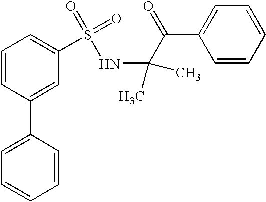 | N-(1,1-dimethyl-2-oxo-2-phenylethyl)biphenyl-3- sulfonamide |
| 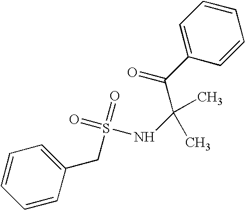 | N-(1,1-dimethyl-2-oxo-2-phenylethyl)-1- phenylmethanesulfonamide |
| 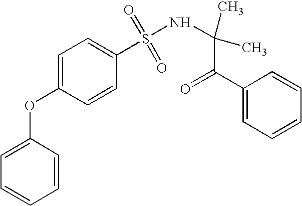 | N-(1,1-dimethyl-2-oxo-2-phenylethyl)-4- phenoxybenzenesulfonamide |
| 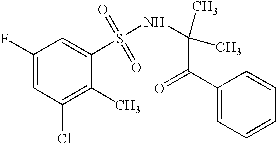 | 3-chloro-N-(1,1-dimethyl-2-oxo-2-phenylethyl)-5-fluoro-2- methylbenzenesulfonamide |
| 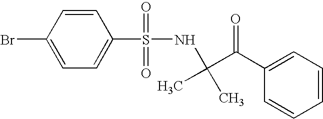 | 4-bromo-N-(1,1-dimethyl-2-oxo-2- phenylethyl)benzenesulfonamide |
| 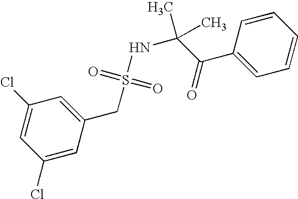 | 1-(3,5-dichlorophenyl)-N-(1,1-dimethyl-2-oxo-2- phenylethyl)methanesulfonamide |
| 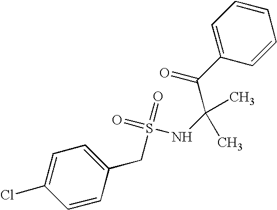 | 1-(4-chlorophenyl)-N-(1,1-dimethyl-2-oxo-2- phenylethyl)methanesulfonamide |
| 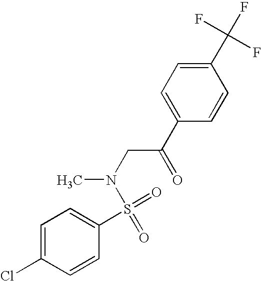 | 4-chloro-N-methyl-N-{2-oxo-2-[4- (trifluoromethyl)phenyl]ethyl}benzenesulfonamide |
| 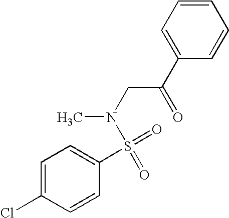 | 4-chloro-N-methyl-N-(2-oxo-2- phenylethyl)benzenesulfonamide |
| 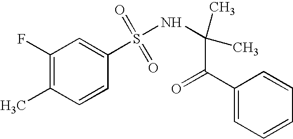 | N-(1,1-dimethyl-2-oxo-2-phenylethyl)-3-fluoro-4- methylbenzenesulfonamide |
| 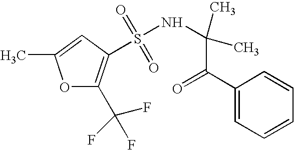 | N-(1,1-dimethyl-2-oxo-2-phenylethyl)-5-methyl-2- (trifluoromethyl)furan-3-sulfonamide |
| 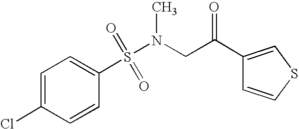 | 4-chloro-N-methyl-N-[2-oxo-2-(3- thienyl)ethyl]benzenesulfonamide |
| 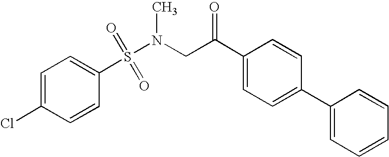 | N-(2-biphenyl-4-yl-2-oxoethyl)-4-chloro-N- methylbenzenesulfonamide |
| 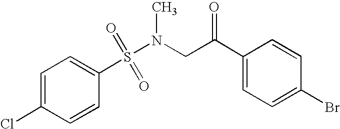 | N-[2-(4-bromophenyl)-2-oxoethyl]-4-chloro-N- methylbenzenesulfonamide |
| 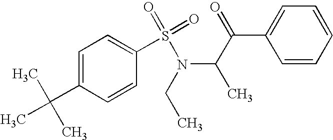 | 4-tert-butyl-N-ethyl-N-(1-methyl-2-oxo-2- phenylethyl)benzenesulfonamide |
| 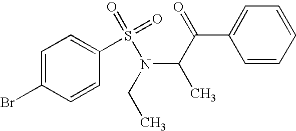 | 4-bromo-N-ethyl-N-(1-methyl-2-oxo-2- phenylethyl)benzenesulfonamide |
| 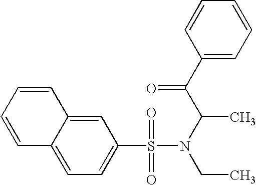 | N-ethyl-N-(1-methyl-2-oxo-2-phenylethyl)naphthalene-2- sulfonamide |
| 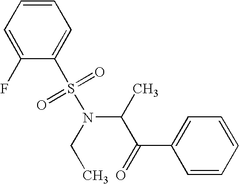 | N-ethyl-2-fluoro-N-(1-methyl-2-oxo-2- phenylethyl)benzenesulfonamide |
| 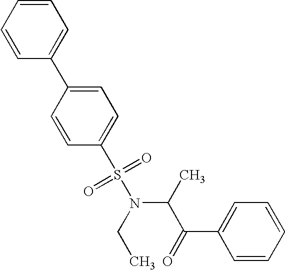 | N-ethyl-N-(1-methyl-2-oxo-2-phenylethyl)biphenyl-4- sulfonamide |
| 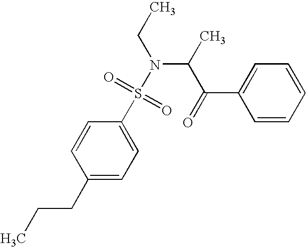 | N-ethyl-N-(1-methyl-2-oxo-2-phenylethyl)-4- propylbenzenesulfonamide |
| 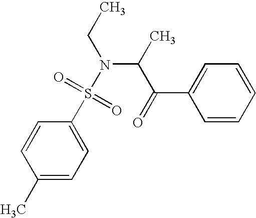 | N-ethyl-4-methyl-N-(1-methyl-2-oxo-2- phenylethyl)benzenesulfonamide |
| 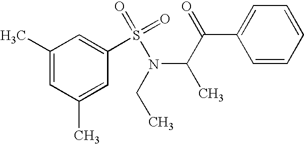 | N-ethyl-3,5-dimethyl-N-(1-methyl-2-oxo-2- phenylethyl)benzenesulfonamide |
| 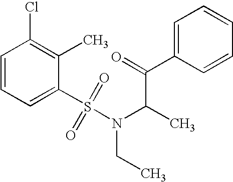 | 3-chloro-N-ethyl-2-methyl-N-(1-methyl-2-oxo-2- phenylethyl)benzenesulfonamide |
| 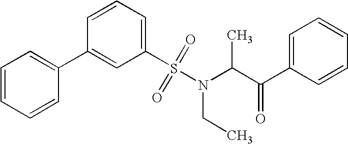 | N-ethyl-N-(1-methyl-2-oxo-2-phenylethyl)biphenyl-3- sulfonamide |
| 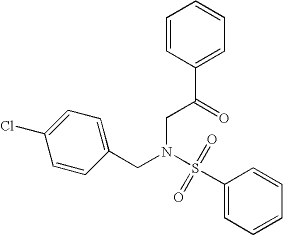 | N-(4-chlorobenzyl)-N-(2-oxo-2- phenylethyl)benzenesulfonamide |
| 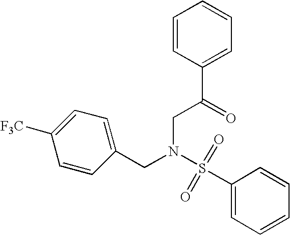 | N-(2-oxo-2-phenylethyl)-N-(4- (trifluoromethyl)benzyl)benzenesulfonamide |
| 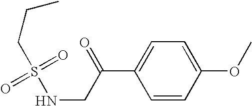 | N-(2-(4-methoxyphenyl)-2-oxoethyl)propane-1- sulfonamide |
| 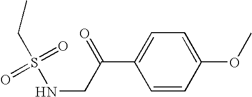 | N-(2-(4-methoxyphenyl)-2-oxoethyl)ethanesulfonamide |
| 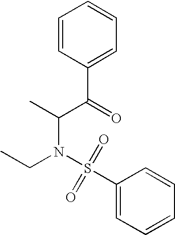 | N-ethyl-N-(1-oxo-1-phenylpropan-2- yl)benzenesulfonamide |
| 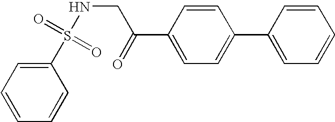 | N-(2-(biphenyl-4-yl)-2-oxoethyl)benzenesulfonamide |
| 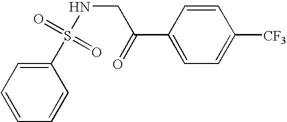 | N-(2-oxo-2-(4- (trifluoromethyl)phenyl)ethyl)benzenesulfonamide |
| 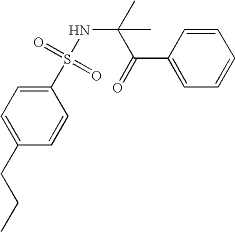 | N-(2-methyl-1-oxo-1-phenylpropan-2-yl)-4- propylbenzenesulfonamide |

### Example 15

Compounds described herein are tested in a cell-based assay using a stable CHO cell line expressing human 11bHSD1. Cells are plated at 20,000 cells/well in 96 well plates and incubated overnight (12-16 hrs) at 37° C./5% CO2. Cells are treated with different concentration of compound in 90 ul serum-free media and incubated for 30 minutes at 37° C./5% CO2. loul of 5 uM cortisone (final concentration 500 nM) is then added to the cells and the plate is incubated at 37° C./5% CO2 for 120 minutes. 15 ul of media is withdrawn and amount of cortisol in the media is measured using the DiscoverX HitHunter Cortisol Assay (DiscoverX corp, CA).

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

A number of embodiments of the invention have been described. Nevertheless, it will be understood that various modifications may be made without departing from the spirit and scope of the invention. Accordingly, other embodiments are within claims.

What is claimed is:

1. A compound of formula (I): 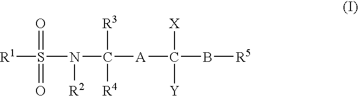
wherein:
each of R¹ and R⁵ is, independently:
(i) C₁-C₂₀ alkyl, optionally substituted with from 1-10 R^{a}; or
(ii) C₃-C₁₆ cycloalkyl, optionally substituted with from 1-10 R^{a}; or
(iii) C₁-C₂₀ haloalkyl or C₃-C₁₆ halocycloalkyl, optionally substituted with from 1-10 R^{a}; or
(iv) C₂-C₂₀ alkenyl, C₂-C₂₀ alkynyl, C₃-C₁₆ cycloalkenyl, heterocyclyl including 3-16 atoms, or heterocycloalkenyl including 3-16 atoms, each of which is optionally substituted with from 1-10 R^{b}; or
(v) C₇-C₂₀ aralkyl, heteroaralkyl including 6-20 atoms, C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c}; or
(vi) C₆-C₁₆ aryl or heteroaryl including 5-16 atoms, each of which is optionally substituted with from 1-10 R^{d}; or
(vii) R¹ together with R³ or R⁴ is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c}; or
(viii) R⁵ together with R³ or R⁴ is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which is optionally substituted with from 1-5 R^{c};
R² is:
(i) hydrogen; or
(ii) C₁-C₂₀ alkyl or C₃-C₁₆ cycloalkyl, each of which is optionally substituted with from 1-10 R^{a}; or
(iii) C₆-C₁₆ aryl, optionally substituted with from 1-10 R^{d}; or
(iv) C₇-C₂₀ aralkyl or heteroaralkyl including 6-20 atoms, each of which is optionally substituted with 1-10 R^{c};
each of R³ and R⁴ is, independently:
(i) hydrogen or C₁-C₁₀ alkyl; or
(ii) R³ and R⁴ together are C₃-C₁₆ cycloalkyl, optionally substituted with from 1-10 R^{a}; C₃-C₁₆ halocycloalkyl; C₃-C₁₆ cycloalkenyl, heterocyclyl including 5-16 atoms, or heterocycloalkenyl including 5-16 atoms, each of which is optionally substituted with from 1-10 R^{b}; or C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c};
(iii) one of R³ or R⁴ is hydrogen or C₁-C₁₀ alkyl, and the other together with R¹ is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c};
(iv) one of R³ or R⁴ is hydrogen or C₁-C₁₀ alkyl, and the other together with R⁵ is is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which can be optionally substituted with from 1-5 R^{c};
each of A and B is, independently, a bond or (CR^{e}R^{f})ₘ;
each of X and Y is, independently:
(i) hydrogen, C₁-C₆ alkyl, or hydroxy; or
(ii) X and Y together are oxo;
R^{a} at each occurrence is, independently, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});
R^{b} at each occurrence is, independently, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});
R^{c} at each occurrence is, independently, C₁-C₁₂ alkyl,C₁-C₁₂ haloalkyl, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});
R^{d} at each occurrence is, independently:
(i) halo; NR^{g}R^{h}; nitro; hydroxy; C₁-C₁₂ alkoxy; C₁-C₁₂ haloalkoxy; C₆-C₁₆ aryloxy; mercapto; C₁-C₆ thioalkoxy; C₆-C₁₆ thioaryloxy; cyano; formyl; —C(O)R^{j}, C₁-C₃ alkylenedioxy; —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NR^{m})R^{j}; S(O)ₙR^{p}; or P(O)(OR^{g})(OR^{h}); or
(ii) C₁-C₁₂ alkyl, optionally substituted with from 1-10 R^{a} and/or optionally inserted with from 1-6 heteroatoms selected from the group consisting of nitrogen, oxygen or sulfur; or
(iii) C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which is optionally substituted with from 1-10 R^{b}; or
(iv) C₁-C₁₂ haloalkyl; or
(v) C₇-C₂₀ aralkyl or heteroaralkyl including 6-20 atoms, each of which is optionally substituted with from 1-10 R^{c}; or
(vi) C₆-C₁₆ aryl or heteroaryl including 5-16 atoms, each of which is optionally substituted with C₁-C₁₂ alkyl, C₁-C₁₂ haloalkyl, halo, NR^{g}R^{h}, nitro, hydroxy, C₁-C₁₂ alkoxy, C₁-C₁₂ haloalkoxy, C₆-C₁₆ aryloxy, mercapto, C₁-C₆ thioalkoxy, C₆-C₁₆ thioaryloxy, cyano, formyl, —C(O)R^{j}, C₁-C₃ alkylenedioxy, —C(O)OR^{j}, —OC(O)R^{j}, —C(O)SR^{j}, —SC(O)R^{j}, —C(S)SR^{j}, —SC(S)R^{j}, —C(O)NR^{g}R^{h}, —C(O)NR^{k}C(O)R^{j}, —C(NR^{m})R^{j}, S(O)ₙR^{p}, or P(O)(OR^{g})(OR^{h});
each of R^{e}, R^{f}, and R^{k}, at each occurrence is, independently, hydrogen or C₁-C₁₀ alkyl;
each of R^{g}, R^{h}, and R^{j}, at each occurrence is, independently, hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; or heteroaryl including 5-16 atoms;
R^{m} is hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms; NR^{g}R^{h}, or OR^{j};
R^{p} is C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms;NR^{g}R^{h}, or OR^{j};
m is 1-20; and
n is 1 or 2;
provided that when R¹ is isopropyl and X and Y together are oxo, the R⁵ is not 4-bromophenyl, 4-benzamidophenyl, 4-methyl-phenyl, 4-isopropylphenyl, 4-isobutylphenyl, 4-t-butylphenyl, 4-methoxyphenyl, 4-isopropoxyphenyl, 4-cyclopentylphenyl, 4-cyclohexylphenyl, 4-(2-furyl)phenyl, 4-(3-furyl)phenyl, 4-(2-thienyl)phenyl, 4-(3-thienyl)phenyl, 4-(pyrrolidin-1-yl)phenyl, 4-(piperidin-1-yl)phenyl, 3-chloro-4-piperidin-1-ylphenyl, 4-(2-fluorophenyl)phenyl, 4-(3-fluorophenyl)phenyl, 4-(2-formylphenyl)phenyl, 4-(3-formylphenyl)phenyl, 4-(4-formylphenyl)phenyl, 4-(4-methylphenyl)phenyl, 4-(4-hydroxphenyl)phenyl, 4-(2-methoxyphenyl)phenyl or 4-(4-methoxyphenyl)phenyl; or a pharmaceutically acceptable salt therof.

2. The compound of claim 1, wherein R¹ is C₆-C₁₆ aryl, optionally substituted with from 1-10 R^{d}; heteroaryl including 5-16 atoms, optionally substituted with from 1-10 R^{d}; C₇-C₂₀ aralkyl, optionally substituted with from 1-10 R^{c}; or C₁-C₂₀ alkyl.

3. The compound of claim 2, wherein R¹ is C₆-C₁₀ aryl, optionally substituted with from 1-3 R^{d}.

4. The compound of claim 3, wherein R¹ is naphthyl.

5. The compound of claim 3, wherein R¹ is phenyl, optionally substituted with from 1-3 R^{d}.

6. The compound of claim 5, wherein R^{d}, at each occurrence is, independently, C₁-C₆ alkyl, C₆-C₁₀ aryl, C₁-C₆ alkoxy, halo, C₆-C₁₀ aryloxy, or nitro.

7. The compound of claim 6, wherein R¹ is phenyl, 4-tert-butylphenyl, 4-biphenyl, 4-chlorophenyl, 3,5-dimethylphenyl, 4-bromophenyl, or 2-fluorophenyl.

8. The compound of claim 2, wherein R¹ is heteroaryl including 5-10 atoms, optionally substituted with from 1-2 R^{d}.

9. The compound of claim 8, wherein R¹ is thienyl, furyl, imidazolyl, or isoxazolyl, optionally substituted with from 1-2 R^{d}.

10. The compound of claim 9, wherein R^{d}, at each occurrence is, independently, C₁-C₆ alkyl, C₁-C₆ haloalkyl, or halo.

11. The compound of claim 2, wherein R¹ is C₇-C₁₀ aralkyl, optionally substituted with from 1-2 R^{c}.

12. The compound of claim 11, wherein R¹ is benzyl or 2-phenylethyl, optionally substituted with halo.

13. The compound of claim 2, wherein R¹ is C₁-C₁₂ alkyl.

14. The compound of claim 13, wherein R¹ is methyl, ethyl, propyl, or isopropyl.

15. The compound of claim 1, wherein R⁵ is C₆-C₁₆ aryl, optionally substituted with from 1-10 R^{d}; or heteroaryl including 5-16 atoms, optionally substituted with from 1-10 R^{d}.

16. The compound of claim 15, wherein R⁵ is C₆-C₁₀ aryl, optionally substituted with from 1-2 R^{d}.

17. The compound of claim 16, wherein R⁵ is naphthyl.

18. The compound of claim 16, wherein R⁵ is phenyl, 4-biphenyl, 4-trifluoromethylphenyl, or 4-methoxyphenyl.

19. The compound of claim 15, wherein R⁵ is heteroaryl including 5-10 atoms, optionally substituted with from 1-2 R^{d}.

20. The compound of claim 19, wherein R⁵ is thienyl, benzothienyl, furyl, imidazolyl, or isoxazolyl, optionally substituted with from 1-2 R^{d}.

21. The compound of claim 20, wherein R^{d}, at each occurrence is, independently, C₁-C₆ alkyl, C₁-C₆ haloalkyl, or halo.

22. The compound of claim 1, wherein R² is hydrogen.

23. The compound of claim 1, wherein R is C₁-C₂₀ alkyl or C₃-C₁₆ cycloalkyl, each of which is optionally substituted with from 1-10 R^{a}; C₆-C₁₆ aryl, optionally substituted with from 1-10 R^{d}; or C₇-C₂₀ aralkyl or heteroaralkyl including 6-20 atoms, each of which is optionally substituted with 1-5 R^{c}.

24. The compound of claim 23, wherein R² is methyl or ethyl.

25. The compound of claim 23, wherein R² is C₇-C₁₀ aralkyl, optionally substituted with oxo or C₁-C₄ haloalkyl.

26. The compound of claim 1, wherein each of A and B is a bond.

27. The compound of claim 1, wherein X and Y together are oxo.

28. The compound of claim 1, wherein each of R³ and R⁴ is, independently: hydrogen or C₁-C₁₀ alkyl.

29. The compound of claim 28, wherein each of R³ and R⁴ is hydrogen.

30. The compound of claim 28, wherein each of R³ and R⁴ is C₁-C₆ alkyl.

31. The compound of claim 30, wherein each of R³ and R⁴ is methyl.

32. The compound of claim 28, wherein one of R³ or R⁴ is hydrogen, and the other is C₁-C₆ alkyl.

33. The compound of claim 32, wherein one of R³ or R⁴ is hydrogen, and the other is methyl or isopropyl.

34. The compound of claim 1, wherein one of R³ or R⁴ is hydrogen or C₁-C₁₀ alkyl, and the other together with R¹ is heterocyclyl including 3-10 atoms or heterocycloalkenyl including 5-10 atoms, each of which can be optionally substituted with from 1-5 R^{b}; or arylheterocyclyl including 8-12 atoms or arylheterocycloalkenyl including 8-12 atoms, each of which can be optionally substituted with from 1-5 R^{c}.

35. The compound of claim 34, wherein one of R³ or R⁴ is hydrogen, and the other together with R¹ is arylheterocyclyl including 9-12 atoms.

36. The compound of claim 1, wherein one of R³ or R⁴ is hydrogen or C₁-C₁₀ alkyl, and the other together with R⁵ is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which is optionally substituted with from 1-5 R^{c}.

37. The compound of claim 36, wherein one of R³ or R⁴ is hydrogen, and the other together with R¹ is C₈-C₁₂ arylcycloalkyl.

38. The compound of claim 1, wherein each of R³ and R⁴ is, independently, hydrogen or C₁-C₁₀ alkyl, each of A and B is a bond and X and Y together are oxo.

39. The compound of claim 38, wherein each of R³ and R⁴ is hydrogen.

40. The compound of claim 38, wherein each of R³ and R⁴ is methyl.

41. The compound of claim 38, wherein one of R³ or R⁴ is hydrogen, and the other is methyl or isopropyl.

42. The compound of claim 38, wherein R² is hydrogen.

43. The compound of claim 38, wherein R² is methyl or ethyl.

44. The compound of claim 38, wherein R² is C₇-C₁₀ aralkyl, optionally substituted with oxo or C₁-C₄ haloalkyl.

45. The compound of claim 38, wherein R¹ is C₆-C₁₀ aryl, optionally substituted with from 1-2 R^{d}.

46. The compound of claim 45, wherein R¹ is naphthyl.

47. The compound of claim 45, wherein R¹ is phenyl, optionally substituted with from 1-2 R^{d}.

48. The compound of claim 47, wherein R^{d}, at each occurrence is, independently, C₁-C₆ alkyl, C₆-C₁₀ aryl, C₁-C₆ alkoxy, halo, C₆-C₁₀ aryloxy, cyano, or nitro.

49. The compound of claim 48, wherein R¹ is phenyl, 4-tert-butylphenyl, 4-biphenyl, 4-chlorophenyl, 3,5-dimethylphenyl, 4-bromophenyl, or 2-fluorophenyl.

50. The compound of claim 38, wherein R¹ is heteroaryl including 5-10 atoms, optionally substituted with from 1-2 R^{d}.

51. The compound of claim 50, wherein R¹ is thienyl, furyl, imidazolyl, or isoxazolyl, optionally substituted with from 1-2 R^{d}.

52. The compound of claim 51, wherein R^{d}, at each occurrence is, independently, C₁-C₆ alkyl, C₁-C₆ haloalkyl, or halo.

53. The compound of claim 38, wherein R¹ is C₇-C₁₀ aralkyl, optionally substituted with from 1-2 R^{c}.

54. The compound of claim 53, wherein R¹ is benzyl or 2-phenylethyl, optionally substituted with halo.

55. The compound of claim 38, wherein R¹ is C₁-C₁₂ alkyl.

56. The compound of claim 55, wherein R¹ is methyl, ethyl, or propyl.

57. The compound of claim 55, wherein R¹ is isopropyl.

58. The compound of claim 38, wherein R⁵ is C₆-C₁₆ aryl, optionally substituted with from 1-10 R^{d}; or heteroaryl including 5-16 atoms, optionally substituted with from 1-10 R^{d}.

59. The compound of claim 58, wherein R⁵ is C₆-C₁₀ aryl, optionally substituted with from 1-2 R^{d}.

60. The compound of claim 59, wherein R⁵ is naphthyl.

61. The compound of claim 59, wherein R⁵ is phenyl, 4-biphenyl, or 4-trifluoromethylphenyl.

62. The compound of claim 59, wherein R⁵ is 4-methoxyphenyl.

63. The compound of claim 58, wherein R⁵ is heteroaryl including 5-10 atoms, optionally substituted with from 1-2 R^{d}.

64. The compound of claim 63, wherein R⁵ is thienyl, benzothienyl, furyl, imidazolyl, or isoxazolyl, optionally substituted with from 1-2 R^{d}.

65. The compound of claim 64, wherein R^{d}, at each occurrence is, independently, C₁-C₆ alkyl, C₁-C₆ haloalkyl, or halo.

66. A pharmaceutical composition comprising an effective amount of a compound of formula (I): 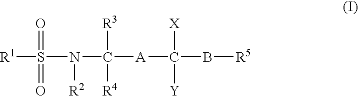
wherein:
each of R¹ and R⁵ is, independently:
(i) C₁-C₂₀ alkyl, optionally substituted with from 1-10 R^{a}; or
(ii) C₃-C₁₆ cycloalkyl, optionally substituted with from 1-10 R^{a}; or
(iii) C₁-C₂₀ haloalkyl or C₃-C₁₆ halocycloalkyl, optionally substituted with from 1-10 R^{a}; or
(iv) C₂-C₂₀ alkenyl, C₂-C₂₀ alkynyl, C₃-C₁₆ cycloalkenyl, heterocyclyl including 3-16 atoms, or heterocycloalkenyl including 3-16 atoms, each of which is optionally substituted with from 1-10 R^{b}; or
(v) C₇-C₂₀ aralkyl, heteroaralkyl including 6-20 atoms, C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c}; or
(vi) C₆-C₁₆ aryl or heteroaryl including 5-16 atoms, each of which is optionally substituted with from 1-10 R^{d}; or
(vii) R¹ together with R³ or R⁴ is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c}; or
(viii) R⁵ together with R³ or R⁴ is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which is optionally substituted with from 1-5 R^{c};
R² is:
(i) hydrogen; or (ii) C₁-C₂₀ alkyl or C₃-C₁₆ cycloalkyl, each of which is optionally substituted with from 1-10 R^{a}; or
(iii) C₆-C₁₆ aryl, optionally substituted with from 1-10 R^{d}; or
(iv) C₇-C₂₀ aralkyl or heteroaralkyl including 6-20 atoms, each of which is optionally substituted with 1-10 R^{c};
each of R³ and R⁴ is, independently:
(i) hydrogen or C₁-C₁₀ alkyl; or
(ii) R³ and R⁴ together are C₃-C₁₆ cycloalkyl, optionally substituted with from 1-10 R^{a}; C₃-C₁₆ halocycloalkyl; C₃-C₁₆ cycloalkenyl, heterocyclyl including 5-16 atoms, or heterocycloalkenyl including 5-16 atoms, each of which is optionally substituted with from 1-10 R^{b}; or C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c};
(iii) one of R³ or R⁴ is hydrogen or C₁-C₁₀ alkyl, and the other together with R¹ is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c};
(iv) one of R³ or R⁴ is hydrogen or C₁-C₁₀ alkyl, and the other together with R⁵ is is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which can be optionally substituted with from 1-5 R^{c};
each of A and B is, independently, a bond or (CR^{e}R^{f})ₘ;
each of X and Y is, independently:
(i) hydrogen, C₁-C₆ alkyl, or hydroxy; or
(ii) X and Y together are oxo;
R^{a} at each occurrence is, independently, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});
R at each occurrence is, independently, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});
R^{c} at each occurrence is, independently, C₁-C₁₂ alkyl, C₁-C₁₂ haloalkyl, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR⁸)(OR^{h});
R^{d} at each occurrence is, independently:
(i) halo; NR^{g}R^{h}; nitro; hydroxy; C₁-C₁₂ alkoxy; C₁-C₁₂ haloalkoxy; C₆-C₁₆ aryloxy; mercapto; C₁-C₆ thioalkoxy; C₆-C₁₆ thioaryloxy; cyano; formyl; —C(O)R^{j}, C₁-C₃ alkylenedioxy; —C(O)OR^{j}; —OC(O)R^{j}; —C(O)SRⁱ; —SC(O)R^{j}; —C(S)SR^{j}; —SC(S)R^{j}; —C(O)NR^{g}R^{h}; —NR^{k}C(O)R^{j}; —C(NR^{m})R^{j}; S(O)ₙR^{p}; or P(O)(OR^{g})(OR^{h}); or
(ii) C₁-C₁₂ alkyl, optionally substituted with from 1-10 R^{a} and/or optionally inserted with from 1-6 heteroatoms selected from the group consisting of nitrogen, oxygen or sulfur; or
(iii) C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which is optionally substituted with from 1-10 R^{b}; or
(iv) C₁-C₁₂ haloalkyl; or
(v) C₇-C₂₀ aralkyl or heteroaralkyl including 6-20 atoms, each of which is optionally substituted with from 1-10 R^{c}; or
(vi) C₆-C₁₆ aryl or heteroaryl including 5-16 atoms, each of which is optionally substituted with C₁-C₁₂ alkyl, C₁-C₁₂ haloalkyl, halo, NR^{g}R^{h}, nitro, hydroxy, C₁-C₁₂ alkoxy, C₁-C₁₂ haloalkoxy, C₆-C₁₆ aryloxy, mercapto, C₁-C₆ thioalkoxy, C₆-C₁₆ thioaryloxy, cyano, formyl, —C(O)R^{j}, C₁-C₃ alkylenedioxy, —C(O)OR^{j}, —OC(O)R^{j}, —C(O)SR^{j}, —SC(O)R^{j}, —C(S)SR^{j}, —SC(S)R^{j}, —C(O)NR^{g}R^{h}, —C(O)NR^{k}C(O)R, —C(NR^{m})R^{j}, S(O)ₙR^{p}, or P(O)(OR^{g})(OR^{h});
each of R^{e}, R^{f}, and R^{k}, at each occurrence is, independently, hydrogen or C₁-C₁₀ alkyl;
each of R^{g}, R^{h}, and R^{j}, at each occurrence is, independently, hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; or heteroaryl including 5-16 atoms;
R^{m} is hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms; NR^{g}R^{h}, or OR^{j};
R^{p} is C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms; NR^{g}R^{h}, or OR^{j};
m is 1-20; and
n is 1 or 2;
provided that when R¹ is isopropyl and X and Y together are oxo, the R⁵ is not 4-bromophenyl, 4-benzamidophenyl, 4-methyl-phenyl, 4-isopropylphenyl, 4-isobutylphenyl, 4-t-butylphenyl, 4-methoxyphenyl, 4-isopropoxyphenyl, 4-cyclopentylphenyl, 4-cyclohexylphenyl, 4-(2-furyl)phenyl, 4-(3-furyl)phenyl, 4-(2-thienyl)phenyl, 4-(3-thienyl)phenyl, 4-(pyrrolidin-1-yl)phenyl, 4-(piperidin-1-yl)phenyl, 3-chloro-4-piperidin-1-ylphenyl, 4-(2-fluorophenyl)phenyl, 4-(3-fluorophenyl)phenyl, 4-(2-formylphenyl)phenyl, 4-(3-formylphenyl)phenyl, 4-(4-formylphenyl)phenyl, 4-(4-methylphenyl)phenyl, 4-(4-hydroxphenyl)phenyl, 4-(2-methoxyphenyl)phenyl or 4-(4-methoxyphenyl)phenyl; or a pharmaceutically acceptable salt therof; and a pharmaceutically acceptable carrier.

67. A method for treating a disease or condition mediated by excess or uncontrolled amounts of cortisol and/or other corticosteroids, the method comprising administering to a subject in need thereof an effective amount of a compound of formula (I): 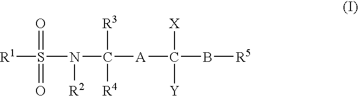
wherein:
each of R¹ and R⁵ is, independently:
(i) C₁-C₂₀ alkyl, optionally substituted with from 1-10 R^{a} and/or optionally inserted with from 1-10 heteroatoms selected from the group consisting of nitrogen, oxygen or sulfur; or
(ii) C₃-C₁₆ cycloalkyl, optionally substituted with from 1-10 R^{a}; or
(iii) C₁-C₂₀ haloalkyl or C₃-C₁₆ halocycloalkyl, optionally substituted with from 1-10 R^{a}; or
(iv) C₂-C₂₀ alkenyl, C₂-C₂₀ alkynyl, C₃-C₁₆ cycloalkenyl, heterocyclyl including 3-16 atoms, or heterocycloalkenyl including 3-16 atoms, each of which is optionally substituted with from 1-10 R^{b}; or
(v) C₇-C₂₀ aralkyl, heteroaralkyl including 6-20 atoms, C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c}; or
(vi) C₆-C₁₆ aryl or heteroaryl including 5-16 atoms, each of which is optionally substituted with from 1-10 R^{d}; or
(vii) R¹ together with R³ or R⁴ is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c}; or
(viii) R⁵ together with R³ or R⁴ is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which is optionally substituted with from 1-5 R^{c};
R² is:
(i) hydrogen; or
(ii) C₁-C₂₀ alkyl or C₃-C₁₆ cycloalkyl, each of which is optionally substituted with from 1-10 R^{a}; or
(iii) C₆-C₁₆ aryl, optionally substituted with from 1-10 R^{d}; or
(iv) C₇-C₂₀ aralkyl or heteroaralkyl including 6-20 atoms, each of which is optionally substituted with 1-10 R^{c};
each of R³ and R⁴ is, independently:
(i) hydrogen or C₁-C₁₀ alkyl; or
(ii) R³ and R⁴ together are C₃-C₁₆ cycloalkyl, optionally substituted with from 1-10 R^{a}; C₃-C₁₆ halocycloalkyl; C₃-C₁₆ cycloalkenyl, heterocyclyl including 5-16 atoms, or heterocycloalkenyl including 5-16 atoms, each of which is optionally substituted with from 1-10 R^{b}; or C₈-C₂₀ arylcycloalkyl, C₈-C₂₀ arylcycloalkenyl, arylheterocyclyl including 8-20 atoms, or arylheterocycloalkenyl including 8-20 atoms, each of which is optionally substituted with from 1-10 R^{c};
(iii) one of R³ or R⁴ is hydrogen or C₁-C₁₀ alkyl, and the other together with R¹ is heterocyclyl including 3-10 atoms or heterocycloalkenyl including 5-10 atoms, each of which is optionally substituted with from 1-5 R^{b}; or arylheterocyclyl including 8-12 atoms or arylheterocycloalkenyl including 8-12 atoms, each of which is optionally substituted with from 1-5 R^{c};
(iv) one of R³ or R⁴ is hydrogen or C₁-C₁₀ alkyl, and the other together with R⁵ is is C₃-C₁₀ cycloalkyl, optionally substituted with from 1-5 R^{a}; C₃-C₁₀ halocycloalkyl; C₃-C₁₀ cycloalkenyl, heterocyclyl including 5-10 atoms, or heterocycloalkenyl including 5-10 atoms, each of which is optionally substituted with from 1-5 R^{b}; or C₈-C₁₂ arylcycloalkyl, C₈-C₁₂ arylcycloalkenyl, arylheterocyclyl including 8-12 atoms, or arylheterocycloalkenyl including 8-12 atoms, each of which can be optionally substituted with from 1-5 R^{c};
each of A and B is, independently, a bond or (CR^{e}R^{f})ₘ;
each of X and Y is, independently:
(i) hydrogen, C₁-C₆ alkyl, or hydroxy; or
(ii) X and Y together are oxo;
R^{a} at each occurrence is, independently, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});
R^{b} at each occurrence is, independently, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; —NR^{k}C(O)R^{j}, —C(NR^{m})R^{j}, S(O)ₙR^{p}, or P(O)(OR^{g})(OR^{h});
R^{c} at each occurrence is, independently, C₁-C₁₂ alkyl, C₁-C₁₂ haloalkyl, halo, NR^{g}R^{h}, nitro, hydroxy, oxo, thioxo, C₁-C₁₂ alkoxy, C₁-C₁₂ haloalkoxy, C₆-C₁₆ aryloxy, mercapto, C₁-C₁₂ thioalkoxy, C₆-C₁₆ thioaryloxy, cyano, formyl, —C(O)R^{j}, —C(O)OR^{j}, —OC(O)R^{j}, —C(O)SR^{j}, —SC(O)R^{j}, —C(S)SR^{j}, —SC(S)R^{j}, —C(O)NR^{g}R^{h}; NR^{k}C(O)R^{j}, —C(NR^{m})R^{j}, S(O)ₙR^{p}, or P(O)(OR^{g})(OR^{h});
R^{d} at each occurrence is, independently:
(i) halo; NR^{g}R^{h}; nitro; hydroxy; C₁-C₁₂ alkoxy; C₁-C₁₂ haloalkoxy; C₆-C₁₆ aryloxy; mercapto; C₁-C₆ thioalkoxy; C₆-C₁₆ thioaryloxy; cyano; formyl; —C(O)R^{j}, C₁-C₃ alkylenedioxy; —C(O)OR^{j}; —OC(O)R^{j}; —C(O)SR^{j}; —SC(O)R^{j}; —C(S)SR^{j}; —SC(S)Rⁱ; —C(O)NR^{g}R^{h}; NR^{k}C(O)Rⁱ; —C(NR^{m})R^{j}; S(O)ₙR^{p}; or P(O)(OR^{g})(OR^{h}); or
(ii) C₁-C₁₂ alkyl, optionally substituted with from 1-10 R^{a} and/or optionally inserted with from 1-6 heteroatoms selected from the group consisting of nitrogen, oxygen or sulfur; or
(iii) C₂-C₂₀ alkenyl, C₂-C₂₀ alkynyl, C₃-C₁₆ cycloalkyl, C₃-C₁₆ cycloalkenyl, heterocyclyl including 3-16 atoms, or heterocycloalkenyl including 3-16 atoms, each of which is optionally substituted with from 1-10 R^{b}; or
(iv) C₁-C₁₂ haloalkyl; or
(v) C₇-C₂₀ aralkyl or heteroaralkyl including 6-20 atoms, each of which is optionally substituted with from 1-10 R^{c}; or
(vi) C₆-C₁₆ aryl or heteroaryl including 5-16 atoms, each of which is optionally substituted with C₁-C₁₂ alkyl, C₁-C₁₂ haloalkyl, halo, NR^{g}R^{h}, nitro, hydroxy, C₁-C₁₂ alkoxy, C₁-C₁₂ haloalkoxy, C₆-C₁₆ aryloxy, mercapto, C₁-C₆ thioalkoxy, C₆-C₁₆ thioaryloxy, cyano, formyl, —C(O)R^{j}, C₁-C₃ alkylenedioxy, —C(O)OR^{j}, —OC(O)R^{j}, —C(O)SR^{j}, —SC(O)R^{j}, —C(S)SR^{j}, —SC(S)R^{j}, —C(O)NR^{g}R^{h}, —NR^{k}C(O)R^{j}, —C(NR^{m})R^{j}, S(O)ₙR^{p}, or P(O)(OR^{g})(OR^{h});
each of R^{e}, R^{f}, and R^{k}, at each occurrence is, independently, hydrogen or C₁-C₁₀ alkyl;
each of R^{g}, R^{h}, and R^{j}, at each occurrence is, independently, hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; or heteroaryl including 5-16 atoms;
R^{m} is hydrogen; C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms; NR^{g}R^{h}, or OR^{j};
R^{p} is C₁-C₁₂ alkyl optionally inserted with from 1-6 heteroatoms selected from the group consisting of nitrogen, oxygen or sulfur; C₂-C₂₀ alkenyl; C₂-C₂₀ alkynyl; C₇-C₂₀ aralkyl; heteroaralkyl including 6-20 atoms; C₃-C₁₆ cycloalkyl; C₃-C₁₆ cycloalkenyl; heterocyclyl including 3-16 atoms; heterocycloalkenyl including 3-16 atoms; C₈-C₂₀ arylcycloalkyl; C₈-C₂₀ arylcycloalkenyl; arylheterocyclyl including 8-20 atoms; or arylheterocycloalkenyl including 8-20 atoms; C₆-C₁₆ aryl; heteroaryl including 5-16 atoms; NR^{g}R^{h}, or OR^{j};
m is 1-20; and
n is 1 or 2; or a pharmaceutically acceptable salt therof.
